SEQUENCE LISTING (45 sequences)

SEQ ID NO: 1 - DNA, 34980 bp, Aureobasidium pullulans
  CDS (1)..(34980)
  misc_feature (1)..(34980): aba1 gene, complete orf.
atgtcgcgaatgccacagggcgcagcaagacgcaacgactgtgtctcggagcaccaaggcactaccgatctggaggatattgtgcgattctgggaacgacacttagacggtgtgaatgcatctgcattccctgctctgtcatctagcttggttgtacctaaacccaaattgcagacagagcatcgcatcagcctcggaaccgccgtgtctgatcagtggtcagatgcagtcatctgtcgagctgcacttgctgtcattttggcccgttatacgcacgctactgaagcgctctacggcattgtggtcgagcagccttcagtctccaatgcccagaaacgatccgccgatgatgcatcctccattgttgtaccgattcgtgtgcaatgtgcatctggtcaatttgggaacgatattttggctgcaattgctactcacgacgcttcttgtcgtagcctcagcgcgatcggcctggatggcattcgctgtcttgatgatgctaaaactgtggctcggggattacagactgtattgactgtaaccagcaggaagtcggtggacgcatcaagcccaaacattctcgacttggagaacatcgcatcttctcacggtcgagctctcatgatagaatgtcaaatgagcaccacctcggcatgcttgcgtgcacagtacgacgcgggcatcttgcgtaatgaacaggtagttcgtcttctcaaacagctcgcgctttcgatccagcactttcgaggtaacgctgccaacgacctgctacgcgacttctgctttatctcgccaggcgaagagatggaaattgcatactggaatcgtcgaagcattcgcacaaatgaggtttgtatccatgatgtgatctttaagagggcgacctacatgccgactgatacggcggtttccgcctgggatggggagtggacatacgcagatctagatgtcgtatcttcatgtcttgccgattacgttcggtccttggatctgaggtctggacaagccataccactatgcttcgagaagtcaagaaacaccatcgccgctatggtggccgttctcaaagctggtcatccgttttgcctgattgacccgtctactccatctgcgagaatcactcagatgtgcgagcagatgtccgctaccgtcgctttcgcttcgagagcactttgtagcatcatgcaagcaggagtctctagatgtattgcagttgatgacgatctctttcaatccttgtcatcagtcatcgggtgtccacagatgtccatgacgagaccccaggaccttgcctatgtcatatttacatccggaagtactggaatcccgaagggcagcatgatcgagcatcgaggttttgcaagctgcgcacttgaattcggacctcaattgttaatcgatcgcaacacgcgtgcattacagttcgcctctcacgcttttggcgcatgcttgttagaggttctggtgacgcttatgcttggaggttgtgtatgcgtcccgtccgaaaacgatcgcttgaacaacctgtcaggtttcattgaacaaagcggcgtgaactggaccctatttacgccttcttttattggagctctcacgcccgagactattcgtggggtgcacactgtcgtgctgggtggagagccaatgacaccattcatcagagacgtatgggcatcaaaagtgcaactcttgtccatatatggacaaagtgagagctcgactgtgtgtagtgtggttaaaatcaagcctgataccaccgatctgagtagcctgggccacgctataggagctcgcttctggatcgttgatgctgaaaatccgagtcgattggcaccaatcggctgcatcggcgagctcatggtagagagtcctggaattgcacgcgaatacctatctgctcaagaagcacagatgtccccattcataacgaagacacctgcttggtatcctatgaagcagcgttgcagtcctgtcaagttctacatgaccggcgatcttgcttgttatggacgtgatggcaccgtcatgaatcttggacgcaaagattcgcaagtcaagatccgaggccaacgcgtggagcttggcgatgtggagactaatctgcgatcagtcttacctaaacacatcatacctgttgtcgaggcgattgattcgatccatgcatccggaagcaaatttctggttgcgatcctgattggcgcaaaccatggaatgaaaaatgaattcgatacagagccaagacgtgaagtctctatactggatgaaaccgcggtgatccgtataaggaagagtatgcaggatcttgttccatcttactgcatacccacacagtatatctgcatggaacgactcctgaccacgacaacagggaaggcggatcgcaagagactacgcgcgatttgcgtggaccttctcaagccttcaaggagagcaatggtaccagaatcttcggacgggcccacgctaaaactcacggcaggacaagttttggatgaggcatggcatcgatacctgcgttttgattctgttctcgatggttctaagtcgaagttctttgatctgaatggagactccatcacagcgatcaagatagcaaatgcggcgaggaaacacggggtaatgctcaaagtagcagacattcttgctaatcctactctcgccgacctgagagctcaatttcagattgatttcacacctcaaaactccatacttcgcacctcgtaccgtggaccaatccaacaatcctttgcgcaaaacaggttgtggtttctggaccagctgaacgttggcgcgtcatggtacatagtaccagtcgcggtgcgcttgcaaggaacagtccatgtcgacgcgcttgtcaccgcactatgtgccctggaacaacgtcatgaaacgttgcgtacgacctttgaagaatccgatggcgagggcatacaacggattcagccaagtgggcttgagcagcttaggttgatcgacgtggattgcgtggactctagggactaccagcgagtattggaagaagagcagacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcatcgccaacgagattcgatgtagagttccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagttcgccgctcgtatcagccgccggctgaatgctatcgtttcggtcaagaatgtcttcgaccaccccgtacctatggatcttgcagcgacaatccaagaaggctcaaagcttcatactccaatccctcgcacggcttacagcggtcctgtcgaacagtctttcgcacaaggacgtctttggttccttgaccaattcaatcctagctcgattgggtatgtgatgcctttcgctgcgcgtcttcatggtcaactacaaatcgaagcgctcacagcagcattgttcgctttggaacagcgacatgagatcctgcgaacaacgttggacgcacacgatggtgtaggcatgcagatcgttcacgcggaacatccgcaacagttgagaatcattgatgtgtcagcaaaggcgtcgagcagttatgctcagacactgcgtgacgagcaggcgtcacctttcgacctaagcaaggaaccaggttggagagtctcgttgctgcagctcagtgagatagattatgttctttccattgtaatgcatcacaccatctatgacggttggtctctcgacgtactccggcgggagctaagtcagttttatgccgctgccatccgtggtcgagaacctctatcgacaatcgagccattgcctatccaataccgcgacttttctgtctggcaaaagcaggaagaccaagtcgcagagcatcgacgacagctccattattggatagagcagctagatggcagctctcctgctgagttcctaaacgataaaccacggcctacgttgctttctggcaaggcaggagttgtggaaattgctgtgaagggcactgtatatcaacgtctgctagagttctgcaggcttcatcaggtcacctcgttcatggtgctgcttgcggcattccgagcgacacactatcgtctgacaggcacagaggacgcgactgtcggaacacccatcgccaatcgcaatcgacctgagctggagaacatgattggattgttcgtgaatactcagtgtatacgcctcaagatcgaggacaatgatactctcgaggagctagtacagcacgttcgtgccacgatcacagcatcaatctcgaaccaggatgtaccctttgaacaggtagtgtctgcattgctaccaggatcacgcgacacctctaggaacccactagttcagctgacttttgcggtgcattctcagcgaaatttggctgacattcagctagaaaacgtggagaccaatgctatgccaatttgcccctcgacacgtttcgacgctgaattccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgtagtatcgtccgcttgatgaggcacagcaatgtctcgagtcgccttctgctacatccccgcatgacccacctgtcgaatctcgccttcgatgcgtcggtgtgggagattttcttgacgctgctcaacggtggaacattgatttgtattgactacctctcgtcactagactgtcgtgctcttggggtaagtatcctggaacaccaggttgacgcatcggtacttcctcctgctttgctcaaacaatgcctagcaaatgtccctgaggcacttgcgagcctgcaagtgctcttgtccgctggagatcgactcgacagtcgtgatgctatagagagttgcgcactcgtgcgcggaagtgtctacaatgggtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttctcatagctcaaacagccgaaaatgaagagctagttggcttcttcacgcttcgacaaacccaggctgtgcagtcaaatggtgccgctggtgttgtgccagagcacagcgactccgagctggcgcaatcctgctcttgcactcaaacggagcgtcgagtccgcaacagattgcaatcctgtcttcctcgctacatggttccgtcgcgaatggtccttttggatcgactgcctgtcaaccccaatggtaaagttgatcgacaagagctcacgaggcgcgctcaggatctcccaataagcgagtcatccccagtgcacgtcaaaccgcgtactgaactggaaaggtcgctgtgcgaggagttcgccgatgttataggtttggaagtcggcgttaccgataatttcttcgacctaggcgggcactctctcatggcgatgaaactcgcagctcgcatcagccgtcgttcgaatgcacatatatcagtcaaggacattttcgaccacccgctgattgcagatctcgcaatgaaaattcgggaaggctccgatctgcacactccaattccccacaggatgtacgttggacctatccagctatcattcgcacagggacgcttgtggttcctcgaccaattgaatttgggcgcatcgtggtacgtcatgccacttgctatgcgcctccaaggctcgctccagctcgacgcgttagagactgcactgtttgctatcgagcagcgacacgaaaccttacggatgacatttgcagaacaagacggagtagctgtacaagtagtgcatgcagcccactacaaacacatcaagatgatcgacaaaccacttagacagaagattgacgtcctgaagatgctggaagaagaacggacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgtagtatcgtccgcttgatgaggcacagcaatgtctcgagtcgccttctgctacatccccgcatgacccacctgtcgaatctcgccttcgatgcgtcggtgtgggagattttcttgacgctgctcaacggtggaacattgatttgtattgactacctctcgtcactagactgtcgtgctcttggggtaagtatcctggaacaccaggttgacgcatcggtacttcctcctgctttgctcaaacaatgcctagcaaatgtccctgaggcacttgcgagcctgcaagtgctcttgtccgctggagatcgactcgacagtcgtgatgctatagagagttgcgcactcgtgcgcggaagtgtctacaatgggtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagcttgccgctcgtatcagccaccgccttcatacacgcatatccgtcaaacacatcttcgatcaccctttgataggcgatttgtctgtccacatagctgactctccggtgcctcttttgacaatcacacgtgcccagcacgctggagcagtggagcagtcattcgcacaagctagattgtggttccttgtccagctaggacttgaatctccttcgtacatcataccaattgtattgcgtttacacggttcactctcaaagactgccattgaaggagctctatcagccctgatggaacgtcatgaggtccttcgtacgacgttcgaggaccataagggtatcggcatgcaagtggtacaagaccatcgtcaccaagacttggttgtaattgacgttgcaggtcaggggtcactcgactacaagcagcacttatacatggagcacgtgaaacctttcgatctgacccgggatcctgggtggagggtagcgctgctgcgtctgggagatgacgaccacgtcctctccatcgtaatgcatcacatcatctccgatggctggtcgattgatatcctgctgcgtgagttgggtcagttctactcggccgcgctccgggggcaggacccgttgtcacagacaagtcctctgccgatccagtatcgtgacttcgctctctggcaaaagcaggatcatcaattagccgatcacgagaagcagctgcggtattgggaagagcaactggcggagagctctccagctgagctgctatgtgatcatgcacgtccgacgacgccctcaggtcaggcaggctcgattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggtgcagttgttgttcgctgttcatgcctatcaagaggtcgaaaatttcgccatccccggtgtgcactccgagttggtgcaaggaacgacctttacaagatttgatgtcgagttccacctgcttgaagaccctgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgcagtatactcagggttgtcacgtctcccccggcccgtgctctgctaccgtccacaatcatcatggcccacctgacaaacattgcattcgatgtatcgctatgggagatatgtacagctcttcttcacggtggtaccctgatctgtattcagtatcttgcctcgctcgatgtcagggggcttcagactacattctctcgcgaagctatcaacgtagctgtgtttcctcctgccttgctaaagacctgtcttgccaagattccatctgctctagcatcgctgagtgccatgttctcgtccggagatcgtctcgactcacgcgatgctagcgagggggccacacttgtgcggcaagggatacacaacgcgtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcgatattgtcccttgattatgtgatcgatgcagccgtccttctgagacagctgattgatcaagagccacaagtggtaggattcgtcattgtatccaccaaacgggcttattcccgacacaacagcggctacgcgtctgaagtttcggcattctgcatcaaagatcagatcgcatggcgcattcgacaacatctctgcaggatgctgccttcctatatggttccctatcaaattgcaattcttgatgaaatgcctatcaatgctaacggcaaggtggatagacagaatcttgcaagcagaactgtcaacgtccaaagaatcctcgccgctccatacatggccccgcgcaatgaagtcgagatttcgctttgcgaacagtatgctgccctgcttgaacacgacgttggcattcttgacgacttcttcgaacttggtggtcactctctcatggctactagactggcctcgcgtatcagctcccgattcagcgctccggtgtctgttcgtgatattttcgaccatccaagaatcatggaccttgctagcatcattcgtgctggagacattcaatggtcccggatactgccttctgcttatgaacgtccagtcgagcaatctttcgcacagaatcgcctgtggttcctgtacaagcttgacataggtacgacacagtataatttaccgctggcgatacaccttcgaggaccactagatatatcagcgctgtttatcgcattcaaggcattgactgaaagacatgaacttttgcgcacaacttttgatgaggatgacggaacatgcctgcagatgttattgcctgaatatcagcatgaagtaaggatcaccgacttgcagggatcacacaaaggtagcctcctggatattctcaacaacaatcagaagactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggtcctcgagatctatgcggcccttctgaacggtggtactgtttactgcattgactatctcactacccttgaccctcacgcgcttgagtctgttttcatcgatgctgatctcaacacggcagtccttcctcccgctctacttaaacaggtccttgcttcgagcccttctaccctccatgcccttgatttactcttcataggaggagatcgattggatgctcgtgacgccctgtacgctaatcgtctggttcgagggtcattatacaatgtctatggcccgacagagaacaccgttctgagcgtcgtttacctctttaatgatgacgatgcatgcattaatggcgtccctatcggccaagtcgtcagtaattccggggtatacgtcatggactcagaacagaaattagtacctcctggggtcatgggagaaatcgtcgtgacaggagacggtctcgcaagagggtatactgactcaaccttaaatactgatcgtttcgttcaaatcagtgtcaacggacgtgtactgcaagcataccgtacaggcgatcgtggtcggtaccgcccgacagacgctcgtcttgagttctttggccgtctagatcaacaaatcaagcttcgcgggcatcgtgtagagctcaaagaaatcgagcaagcgatgcttggccacaatgctgttgatgatgcaggagttgtcgctctggagatatctgagtgccaagagctagagatggttggctttgtgactctacgcaatcttggaaccatggaagcaactaacaatctcgcacacacaagctggaacccagtgactctcaaaacccctttagcatcacaaatagtggctgaggttcggggtagactccagcgaaatctgccactctatatggtacccgctacgattgtggtattacatactatgccagtcaatgccaacgggaagctcgaccgacaagcacttgtgaaagctgcaatgacgcttccaaaaactgctccactggtatggatggctccgcgcaatgaaggagagacatcgctatgtgaggagctaacagatatcttgggggtgaacgtcgggatcaccgataacttttttgaccttggggggcattccctcctggcaaccagagtagccgcgcgaatcagccgacgtcttgatgccctggtgaccgtcaaacaaatattcgaccatccagtcattggagatctcgcagctgcaattcaagggggttcagtacggcatttaccaataactgcaagcgaggtcgatggacctgttcagcagtccttcgcgcaaaatcgcttgtggttcctagagcagatgaatattggagctacttggtacatcgtaccgttagcagtgcgtctgtacggcacactgcgagttgaggctctgaatattgcgttgcgtacgattcagcaacgccacgaaacattacgaacgaccttcgaagaactaaatgggattgccgttcaacgttgtgattcaacctgccaaggccaattaagggtggtagatttagtcgggcaggggccagatcgctatagagagattctggatgtccagcaaactacaccattcgagctgagccaggagcctggatggagggtagcgctgcttcgtctgggagatgacgaccacgtcctctccatcgtcatgcatcacatcatctccgacggttggtctgtggacgtgctgctacgtgagataggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagttcgccgctcgtatcagccgccggctgaatgctatcgtttcggtcaagaatgtcttcgaccaccccgtacctatggatcttgcagcgacaatccaagaaggctcaaagcttcatactccaatccctcgcacggcttacagcggtcctgtcgaacagtctttcgcacaaggacgtctttggttccttgaccaattcaatcctagctcgattgggtatgtgatgcctttcgctgcgcgtcttcatggtcaactacaaatcgaagcgctcacagcagcattgttcgctttggaacagcgacatgagatcctgcgaacaacgttggacgcacacgatggtgtaggcatgcagatcgttcacgcggaacatccgcaacagttgagaatcattgatgtgtcagcaaaggcgtcgagcagttatgctcagacactgcgtgacgagcaggcgtcacctttcgacctaagcaaggaaccaggttggagagtctcgttgctgcagctcagtgagatagattatgttctttccattgtaatgcatcacaccatctatgacggttggtctctcgacgtactccggcgggagctaagtcagttttatgccgctgccatccgtggtcgagaacctctatcgacaatcgagccattgcctatccaataccgcgacttttctgtctggcaaaagcaggaagaccaagtcgcagagcatcgacgacagctccattattggatagagcagctagatggcagctctcctgctgagttcctaaacgataaaccacggcctacgttgctttctggcaaggcaggagttgtggaaattgctgtgaagggcactgtatatcaacgtctgctagagttctgcaggcttcatcaggtcacctcgttcatggtgctgcttgcggcattccgagcgacacactatcgtctgacaggcacagaggacgcgactgtcggaacacccatcgccaatcgcaatcgacctgagctggagaacatgattggattgttcgtgaatactcagtgtatacgcctcaagatcgaggacaatgatactctcgaggagctagtacagcacgttcgtgccacgatcacagcatcaatctcgaaccaggatgtaccctttgaacaggtagtgtctgcattgctaccaggatcacgcgacacctctaggaacccactagttcagctgacttttgcggtgcattctcagcgaaatttggctgacattcagctagaaaacgtggagaccaatgctatgccaatttgcccctcgacacgtttcgacgctgaattccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcaccatcatagacgctcgcgcacttggcgttatctttgcgcaacaaagtatcaacgcaaccatgctgtcacctctactcctcaaacaatttttgtcagatgcaccattcgtgctgcgatctctgcatgccctttatctagggggggacagacttcagggtcgtgacgcaatccaggcttgtcgtgtaggttgcgcatttgtcatcaatgcctatggcccaacagagaattctgtcatcagtactacttacacacttgtgaagggaaatgcggacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttctcatagctcaaacagccgaaaatgaagagctagttggcttcttcacgcttcgacaaacccaggctgtgcagtcaaatggtgccgctggtgttgtgccagagcacagcgactccgagctggcgcaatcctgctcttgcactcaaacggagcgtcgagtccgcaacagattgcaatcctgtcttcctcgctacatggttccgtcgcgaatggtccttttggatcgactgcctgtcaaccccaatggtaaagttgatcgacaagagctcacgaggcgcgctcaggatctcccaataagcgagtcatccccagtgcacgtcaaaccgcgtactgaactggaaaggtcgctgtgcgaggagttcgccgatgttataggtttggaagtcggcgttaccgataatttcttcgacctaggcgggcactctctcatggcgatgaaactcgcagctcgcatcagccgtcgttcgaatgcacatatatcagtcaaggacattttcgaccacccgctgattgcagatctcgcaatgaaaattcgggaaggctccgatctgcacactccaattccccacaggatgtacgttggacctatccagctatcattcgcacagggacgcttgtggttcctcgaccaattgaatttgggcgcatcgtggtacgtcatgccacttgctatgcgcctccaaggctcgctccagctcgacgcgttagagactgcactgtttgctatcgagcagcgacacgaaaccttacggatgacatttgcagaacaagacggagtagctgtacaagtagtgcatgcagcccactacaaacacatcaagatgatcgacaaaccacttagacagaagattgacgtcctgaagatgctggaagaagaacggacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagcttgccgctcgtatcagccgtcaactaaatatccaagtctcagtccgagacatctttgactatcccgttatagtcgacctcacagacagattgagactccatcatacgcgtatccttactcatgatcatggacaacatggacagccagacctcaagccattcaccttgctaccaaccaacaatcctcaagaattcctacagcatcacattttgccacaacttgttcccgatcatgcgaagatcctcgatgtgtatcccgttacaagaatacagagaaggtttcttcatcatccgaagcgcggcctccctcgttttccctccatggtcttctttgacttccctcctggttcagacccacacaagctaagattagcttgtatggcattagtccagcgtttcgacattcttcgcacaatcttcctttctgtttcgggtcaattcttccaagtggtcctggatggatatgggattgtcataccggtcatcgaggttgacgaagagctagacgacgccacccgtaaattacacgattccgatattcagcagcccttacggttgggaaaaccgttaatacgcattgctgtcttgaaaaggcagcactccagagtacgagcagtcttgcgcttgtcgcatgctctctatgatggtttgagctttgagcatatcatccaatctcttcatgccctttatctcgatatcaccctttcggccccaccgaagtttggactctacgtacaacatatgatacaaagtcgcgcagaaggttatgctttctggcggtctgtcttgaagggctcgtcgatgacaattctcgagcgttctagcacccttcaatcgcggcagccgcatcttggacgttttctctctgcggagaaaattattaaggctcctttacacgccaacaagtctggaatcacacaggcaacagtgttcgcggccgcaaacgcactcatgcttgcgaatcttactggtactaatgacgttgtgtttgcccgcattgtctctggacgtcaatctttgcctaagaactttcagcacgttgtgggaccttgcacgaacgatgtgcccgttcgcgtacgcatggagcctggcgtgggaccaaaagctttactcagacaggtgcaagaccagtatgttcatagcttccctttcgaaacactaggattcgacgagatcaaggagaactgtacggactggccagaaagaatcacgaattttgggtgttctacaacttaccagaactttgacatttttcccaaaagtcagattgaccaccagcagattcaaatggctagcttggcaagcgagtatcagaatcgagaaacctgggacgaagcgccgctatacgacctcaatgtcacaggagtacctcagcctgacggacgtcatatcaagatatacgtgggtgtagacgggcagctttgcgatgaaagcacgcttgattgcattctctcggatatttgtgagggtgtggtctcgctcacagacgctttgcaagaacttcccgctgctagcattactgagtag

SEQ ID NO: 2 - protein, 11659 aa, Aureobasidium pullulans
MSRMPQGAARRNDCVSEHQGTTDLEDIVRFWERHLDGVNASAFPALSSSLVVPKPKLQTEHRISLGTAVSDQWSDAVICRAALAVILARYTHATEALYGIVVEQPSVSNAQKRSADDASSIVVPIRVQCASGQFGNDILAAIATHDASCRSLSAIGLDGIRCLDDAKTVARGLQTVLTVTSRKSVDASSPNILDLENIASSHGRALMIECQMSTTSACLRAQYDAGILRNEQVVRLLKQLALSIQHFRGNAANDLLRDFCFISPGEEMEIAYWNRRSIRTNEVCIHDVIFKRATYMPTDTAVSAWDGEWTYADLDVVSSCLADYVRSLDLRSGQAIPLCFEKSRNTIAAMVAVLKAGHPFCLIDPSTPSARITQMCEQMSATVAFASRALCSIMQAGVSRCIAVDDDLFQSLSSVIGCPQMSMTRPQDLAYVIFTSGSTGIPKGSMIEHRGFASCALEFGPQLLIDRNTRALQFASHAFGACLLEVLVTLMLGGCVCVPSENDRLNNLSGFIEQSGVNWTLFTPSFIGALTPETIRGVHTVVLGGEPMTPFIRDVWASKVQLLSIYGQSESSTVCSVVKIKPDTTDLSSLGHAIGARFWIVDAENPSRLAPIGCIGELMVESPGIAREYLSAQEAQMSPFITKTPAWYPMKQRCSPVKFYMTGDLACYGRDGTVMNLGRKDSQVKIRGQRVELGDVETNLRSVLPKHIIPVVEAIDSIHASGSKFLVAILIGANHGMKNEFDTEPRREVSILDETAVIRIRKSMQDLVPSYCIPTQYICMERLLTTTTGKADRKRLRAICVDLLKPSRRAMVPESSDGPTLKLTAGQVLDEAWHRYLRFDSVLDGSKSKFFDLNGDSITAIKIANAARKHGVMLKVADILANPTLADLRAQFQIDFTPQNSILRTSYRGPIQQSFAQNRLWFLDQLNVGASWYIVPVAVRLQGTVHVDALVTALCALEQRHETLRTTFEESDGEGIQRIQPSGLEQLRLIDVDCVDSRDYQRVLEEEQTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVASPTRFDVEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKFAARISRRLNAIVSVKNVFDHPVPMDLAATIQEGSKLHTPIPRTAYSGPVEQSFAQGRLWFLDQFNPSSIGYVMPFAARLHGQLQIEALTAALFALEQRHEILRTTLDAHDGVGMQIVHAEHPQQLRIIDVSAKASSSYAQTLRDEQASPFDLSKEPGWRVSLLQLSEIDYVLSIVMHHTIYDGWSLDVLRRELSQFYAAAIRGREPLSTIEPLPIQYRDFSVWQKQEDQVAEHRRQLHYWIEQLDGSSPAEFLNDKPRPTLLSGKAGVVEIAVKGTVYQRLLEFCRLHQVTSFMVLLAAFRATHYRLTGTEDATVGTPIANRNRPELENMIGLFVNTQCIRLKIEDNDTLEELVQHVRATITASISNQDVPFEQVVSALLPGSRDTSRNPLVQLTFAVHSQRNLADIQLENVETNAMPICPSTRFDAEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSIVRLMRHSNVSSRLLLHPRMTHLSNLAFDASVWEIFLTLLNGGTLICIDYLSSLDCRALGVSILEHQVDASVLPPALLKQCLANVPEALASLQVLLSAGDRLDSRDAIESCALVRGSVYNGYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVLIAQTAENEELVGFFTLRQTQAVQSNGAAGVVPEHSDSELAQSCSCTQTERRVRNRLQSCLPRYMVPSRMVLLDRLPVNPNGKVDRQELTRRAQDLPISESSPVHVKPRTELERSLCEEFADVIGLEVGVTDNFFDLGGHSLMAMKLAARISRRSNAHISVKDIFDHPLIADLAMKIREGSDLHTPIPHRMYVGPIQLSFAQGRLWFLDQLNLGASWYVMPLAMRLQGSLQLDALETALFAIEQRHETLRMTFAEQDGVAVQVVHAAHYKHIKMIDKPLRQKIDVLKMLEEERTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSIVRLMRHSNVSSRLLLHPRMTHLSNLAFDASVWEIFLTLLNGGTLICIDYLSSLDCRALGVSILEHQVDASVLPPALLKQCLANVPEALASLQVLLSAGDRLDSRDAIESCALVRGSVYNGYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKLAARISHRLHTRISVKHIFDHPLIGDLSVHIADSPVPLLTITRAQHAGAVEQSFAQARLWFLVQLGLESPSYIIPIVLRLHGSLSKTAIEGALSALMERHEVLRTTFEDHKGIGMQVVQDHRHQDLVVIDVAGQGSLDYKQHLYMEHVKPFDLTRDPGWRVALLRLGDDDHVLSIVMHHIISDGWSIDILLRELGQFYSAALRGQDPLSQTSPLPIQYRDFALWQKQDHQLADHEKQLRYWEEQLAESSPAELLCDHARPTTPSGQAGSIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLLFAVHAYQEVENFAIPGVHSELVQGTTFTRFDVEFHLLEDPDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSILRVVTSPPARALLPSTIIMAHLTNIAFDVSLWEICTALLHGGTLICIQYLASLDVRGLQTTFSREAINVAVFPPALLKTCLAKIPSALASLSAMFSSGDRLDSRDASEGATLVRQGIHNAYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAILSLDYVIDAAVLLRQLIDQEPQVVGFVIVSTKRAYSRHNSGYASEVSAFCIKDQIAWRIRQHLCRMLPSYMVPYQIAILDEMPINANGKVDRQNLASRTVNVQRILAAPYMAPRNEVEISLCEQYAALLEHDVGILDDFFELGGHSLMATRLASRISSRFSAPVSVRDIFDHPRIMDLASIIRAGDIQWSRILPSAYERPVEQSFAQNRLWFLYKLDIGTTQYNLPLAIHLRGPLDISALFIAFKALTERHELLRTTFDEDDGTCLQMLLPEYQHEVRITDLQGSHKGSLLDILNNNQKTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASVLEIYAALLNGGTVYCIDYLTTLDPHALESVFIDADLNTAVLPPALLKQVLASSPSTLHALDLLFIGGDRLDARDALYANRLVRGSLYNVYGPTENTVLSVVYLFNDDDACINGVPIGQVVSNSGVYVMDSEQKLVPPGVMGEIVVTGDGLARGYTDSTLNTDRFVQISVNGRVLQAYRTGDRGRYRPTDARLEFFGRLDQQIKLRGHRVELKEIEQAMLGHNAVDDAGVVALEISECQELEMVGFVTLRNLGTMEATNNLAHTSWNPVTLKTPLASQIVAEVRGRLQRNLPLYMVPATIVVLHTMPVNANGKLDRQALVKAAMTLPKTAPLVWMAPRNEGETSLCEELTDILGVNVGITDNFFDLGGHSLLATRVAARISRRLDALVTVKQIFDHPVIGDLAAAIQGGSVRHLPITASEVDGPVQQSFAQNRLWFLEQMNIGATWYIVPLAVRLYGTLRVEALNIALRTIQQRHETLRTTFEELNGIAVQRCDSTCQGQLRVVDLVGQGPDRYREILDVQQTTPFELSQEPGWRVALLRLGDDDHVLSIVMHHIISDGWSVDVLLREIGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKFAARISRRLNAIVSVKNVFDHPVPMDLAATIQEGSKLHTPIPRTAYSGPVEQSFAQGRLWFLDQFNPSSIGYVMPFAARLHGQLQIEALTAALFALEQRHEILRTTLDAHDGVGMQIVHAEHPQQLRIIDVSAKASSSYAQTLRDEQASPFDLSKEPGWRVSLLQLSEIDYVLSIVMHHTIYDGWSLDVLRRELSQFYAAAIRGREPLSTIEPLPIQYRDFSVWQKQEDQVAEHRRQLHYWIEQLDGSSPAEFLNDKPRPTLLSGKAGVVEIAVKGTVYQRLLEFCRLHQVTSFMVLLAAFRATHYRLTGTEDATVGTPIANRNRPELENMIGLFVNTQCIRLKIEDNDTLEELVQHVRATITASISNQDVPFEQVVSALLPGSRDTSRNPLVQLTFAVHSQRNLADIQLENVETNAMPICPSTRFDAEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTIIDARALGVIFAQQSINATMLSPLLLKQFLSDAPFVLRSLHALYLGGDRLQGRDAIQACRVGCAFVINAYGPTENSVISTTYTLVKGNADFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVLIAQTAENEELVGFFTLRQTQAVQSNGAAGVVPEHSDSELAQSCSCTQTERRVRNRLQSCLPRYMVPSRMVLLDRLPVNPNGKVDRQELTRRAQDLPISESSPVHVKPRTELERSLCEEFADVIGLEVGVTDNFFDLGGHSLMAMKLAARISRRSNAHISVKDIFDHPLIADLAMKIREGSDLHTPIPHRMYVGPIQLSFAQGRLWFLDQLNLGASWYVMPLAMRLQGSLQLDALETALFAIEQRHETLRMTFAEQDGVAVQVVHAAHYKHIKMIDKPLRQKIDVLKMLEEERTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKLAARISRQLNIQVSVRDIFDYPVIVDLTDRLRLHHTRILTHDHGQHGQPDLKPFTLLPTNNPQEFLQHHILPQLVPDHAKILDVYPVTRIQRRFLHHPKRGLPRFPSMVFFDFPPGSDPHKLRLACMALVQRFDILRTIFLSVSGQFFQVVLDGYGIVIPVIEVDEELDDATRKLHDSDIQQPLRLGKPLIRIAVLKRQHSRVRAVLRLSHALYDGLSFEHIIQSLHALYLDITLSAPPKFGLYVQHMIQSRAEGYAFWRSVLKGSSMTILERSSTLQSRQPHLGRFLSAEKIIKAPLHANKSGITQATVFAAANALMLANLTGTNDVVFARIVSGRQSLPKNFQHVVGPCTNDVPVRVRMEPGVGPKALLRQVQDQYVHSFPFETLGFDEIKENCTDWPERITNFGCSTTYQNFDIFPKSQIDHQQIQMASLASEYQNRETWDEAPLYDLNVTGVPQPDGRHIKIYVGVDGQLCDESTLDCILSDICEGVVSLTDALQELPAASITE

SEQ ID NO: 3 - DNA, 2682 bp, Aureobasidium pullulans
  misc_feature (1)..(2682): aba1,1, CAT(D-Hmp)
atgtcgcgaatgccacagggcgcagcaagacgcaacgactgtgtctcggagcaccaaggcactaccgatctggaggatattgtgcgattctgggaacgacacttagacggtgtgaatgcatctgcattccctgctctgtcatctagcttggttgtacctaaacccaaattgcagacagagcatcgcatcagcctcggaaccgccgtgtctgatcagtggtcagatgcagtcatctgtcgagctgcacttgctgtcattttggcccgttatacgcacgctactgaagcgctctacggcattgtggtcgagcagccttcagtctccaatgcccagaaacgatccgccgatgatgcatcctccattgttgtaccgattcgtgtgcaatgtgcatctggtcaatttgggaacgatattttggctgcaattgctactcacgacgcttcttgtcgtagcctcagcgcgatcggcctggatggcattcgctgtcttgatgatgctaaaactgtggctcggggattacagactgtattgactgtaaccagcaggaagtcggtggacgcatcaagcccaaacattctcgacttggagaacatcgcatcttctcacggtcgagctctcatgatagaatgtcaaatgagcaccacctcggcatgcttgcgtgcacagtacgacgcgggcatcttgcgtaatgaacaggtagttcgtcttctcaaacagctcgcgctttcgatccagcactttcgaggtaacgctgccaacgacctgctacgcgacttctgctttatctcgccaggcgaagagatggaaattgcatactggaatcgtcgaagcattcgcacaaatgaggtttgtatccatgatgtgatctttaagagggcgacctacatgccgactgatacggcggtttccgcctgggatggggagtggacatacgcagatctagatgtcgtatcttcatgtcttgccgattacgttcggtccttggatctgaggtctggacaagccataccactatgcttcgagaagtcaagaaacaccatcgccgctatggtggccgttctcaaagctggtcatccgttttgcctgattgacccgtctactccatctgcgagaatcactcagatgtgcgagcagatgtccgctaccgtcgctttcgcttcgagagcactttgtagcatcatgcaagcaggagtctctagatgtattgcagttgatgacgatctctttcaatccttgtcatcagtcatcgggtgtccacagatgtccatgacgagaccccaggaccttgcctatgtcatatttacatccggaagtactggaatcccgaagggcagcatgatcgagcatcgaggttttgcaagctgcgcacttgaattcggacctcaattgttaatcgatcgcaacacgcgtgcattacagttcgcctctcacgcttttggcgcatgcttgttagaggttctggtgacgcttatgcttggaggttgtgtatgcgtcccgtccgaaaacgatcgcttgaacaacctgtcaggtttcattgaacaaagcggcgtgaactggaccctatttacgccttcttttattggagctctcacgcccgagactattcgtggggtgcacactgtcgtgctgggtggagagccaatgacaccattcatcagagacgtatgggcatcaaaagtgcaactcttgtccatatatggacaaagtgagagctcgactgtgtgtagtgtggttaaaatcaagcctgataccaccgatctgagtagcctgggccacgctataggagctcgcttctggatcgttgatgctgaaaatccgagtcgattggcaccaatcggctgcatcggcgagctcatggtagagagtcctggaattgcacgcgaatacctatctgctcaagaagcacagatgtccccattcataacgaagacacctgcttggtatcctatgaagcagcgttgcagtcctgtcaagttctacatgaccggcgatcttgcttgttatggacgtgatggcaccgtcatgaatcttggacgcaaagattcgcaagtcaagatccgaggccaacgcgtggagcttggcgatgtggagactaatctgcgatcagtcttacctaaacacatcatacctgttgtcgaggcgattgattcgatccatgcatccggaagcaaatttctggttgcgatcctgattggcgcaaaccatggaatgaaaaatgaattcgatacagagccaagacgtgaagtctctatactggatgaaaccgcggtgatccgtataaggaagagtatgcaggatcttgttccatcttactgcatacccacacagtatatctgcatggaacgactcctgaccacgacaacagggaaggcggatcgcaagagactacgcgcgatttgcgtggaccttctcaagccttcaaggagagcaatggtaccagaatcttcggacgggcccacgctaaaactcacggcaggacaagttttggatgaggcatggcatcgatacctgcgttttgattctgttctcgatggttctaagtcgaagttctttgatctgaatggagactccatcacagcgatcaagatagcaaatgcggcgaggaaacacggggtaatgctcaaagtagcagacattcttgctaatcctactctcgccgacctgagagctcaatttcagatt

SEQ ID NO: 4 - protein, 894 aa, Aureobasidium pullulans
  MISC_FEATURE (1)..(895): aba1.1 CAT(D-Hmp)
MSRMPQGAARRNDCVSEHQGTTDLEDIVRFWERHLDGVNASAFPALSSSLVVPKPKLQTEHRISLGTAVSDQWSDAVICRAALAVILARYTHATEALYGIVVEQPSVSNAQKRSADDASSIVVPIRVQCASGQFGNDILAAIATHDASCRSLSAIGLDGIRCLDDAKTVARGLQTVLTVTSRKSVDASSPNILDLENIASSHGRALMIECQMSTTSACLRAQYDAGILRNEQVVRLLKQLALSIQHFRGNAANDLLRDFCFISPGEEMEIAYWNRRSIRTNEVCIHDVIFKRATYMPTDTAVSAWDGEWTYADLDVVSSCLADYVRSLDLRSGQAIPLCFEKSRNTIAAMVAVLKAGHPFCLIDPSTPSARITQMCEQMSATVAFASRALCSIMQAGVSRCIAVDDDLFQSLSSVIGCPQMSMTRPQDLAYVIFTSGSTGIPKGSMIEHRGFASCALEFGPQLLIDRNTRALQFASHAFGACLLEVLVTLMLGGCVCVPSENDRLNNLSGFIEQSGVNWTLFTPSFIGALTPETIRGVHTVVLGGEPMTPFIRDVWASKVQLLSIYGQSESSTVCSVVKIKPDTTDLSSLGHAIGARFWIVDAENPSRLAPIGCIGELMVESPGIAREYLSAQEAQMSPFITKTPAWYPMKQRCSPVKFYMTGDLACYGRDGTVMNLGRKDSQVKIRGQRVELGDVETNLRSVLPKHIIPVVEAIDSIHASGSKFLVAILIGANHGMKNEFDTEPRREVSILDETAVIRIRKSMQDLVPSYCIPTQYICMERLLTTTTGKADRKRLRAICVDLLKPSRRAMVPESSDGPTLKLTAGQVLDEAWHRYLRFDSVLDGSKSKFFDLNGDSITAIKIANAARKHGVMLKVADILANPTLADLRAQFQI

SEQ ID NO: 5 - DNA, 4464 bp, Aureobasidium pullulans
  misc_feature (1)..(4467): aba1.2 CAMT(val)
gatttcacacctcaaaactccatacttcgcacctcgtaccgtggaccaatccaacaatcctttgcgcaaaacaggttgtggtttctggaccagctgaacgttggcgcgtcatggtacatagtaccagtcgcggtgcgcttgcaaggaacagtccatgtcgacgcgcttgtcaccgcactatgtgccctggaacaacgtcatgaaacgttgcgtacgacctttgaagaatccgatggcgagggcatacaacggattcagccaagtgggcttgagcagcttaggttgatcgacgtggattgcgtggactctagggactaccagcgagtattggaagaagagcagacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcatcgccaacgagattcgatgtagagttccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagttcgccgctcgtatcagccgccggctgaatgctatcgtttcggtcaagaatgtcttcgaccaccccgtacctatggatcttgcagcgacaatccaa

SEQ ID NO: 6 - protein, 1488 aa, Aureobasidium pullulans
  MISC_FEATURE (1)..(1488): aba1.2 CAMT(val)
DFTPQNSILRTSYRGPIQQSFAQNRLWFLDQLNVGASWYIVPVAVRLQGTVHVDALVTALCALEQRHETLRTTFEESDGEGIQRIQPSGLEQLRLIDVDCVDSRDYQRVLEEEQTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVASPTRFDVEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKFAARISRRLNAIVSVKNVFDHPVPMDLAATIQ

SEQ ID NO: 7 - DNA, 3255 bp, Aureobasidium pullulans
  misc_feature (1)..(3255): aba1.3 CAT(phe)
gaaggctcaaagcttcatactccaatccctcgcacggcttacagcggtcctgtcgaacagtctttcgcacaaggacgtctttggttccttgaccaattcaatcctagctcgattgggtatgtgatgcctttcgctgcgcgtcttcatggtcaactacaaatcgaagcgctcacagcagcattgttcgctttggaacagcgacatgagatcctgcgaacaacgttggacgcacacgatggtgtaggcatgcagatcgttcacgcggaacatccgcaacagttgagaatcattgatgtgtcagcaaaggcgtcgagcagttatgctcagacactgcgtgacgagcaggcgtcacctttcgacctaagcaaggaaccaggttggagagtctcgttgctgcagctcagtgagatagattatgttctttccattgtaatgcatcacaccatctatgacggttggtctctcgacgtactccggcgggagctaagtcagttttatgccgctgccatccgtggtcgagaacctctatcgacaatcgagccattgcctatccaataccgcgacttttctgtctggcaaaagcaggaagaccaagtcgcagagcatcgacgacagctccattattggatagagcagctagatggcagctctcctgctgagttcctaaacgataaaccacggcctacgttgctttctggcaaggcaggagttgtggaaattgctgtgaagggcactgtatatcaacgtctgctagagttctgcaggcttcatcaggtcacctcgttcatggtgctgcttgcggcattccgagcgacacactatcgtctgacaggcacagaggacgcgactgtcggaacacccatcgccaatcgcaatcgacctgagctggagaacatgattggattgttcgtgaatactcagtgtatacgcctcaagatcgaggacaatgatactctcgaggagctagtacagcacgttcgtgccacgatcacagcatcaatctcgaaccaggatgtaccctttgaacaggtagtgtctgcattgctaccaggatcacgcgacacctctaggaacccactagttcagctgacttttgcggtgcattctcagcgaaatttggctgacattcagctagaaaacgtggagaccaatgctatgccaatttgcccctcgacacgtttcgacgctgaattccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgtagtatcgtccgcttgatgaggcacagcaatgtctcgagtcgccttctgctacatccccgcatgacccacctgtcgaatctcgccttcgatgcgtcggtgtgggagattttcttgacgctgctcaacggtggaacattgatttgtattgactacctctcgtcactagactgtcgtgctcttggggtaagtatcctggaacaccaggttgacgcatcggtacttcctcctgctttgctcaaacaatgcctagcaaatgtccctgaggcacttgcgagcctgcaagtgctcttgtccgctggagatcgactcgacagtcgtgatgctatagagagttgcgcactcgtgcgcggaagtgtctacaatgggtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttctcatagctcaaacagccgaaaatgaagagctagttggcttcttcacgcttcgacaaacccaggctgtgcagtcaaatggtgccgctggtgttgtgccagagcacagcgactccgagctggcgcaatcctgctcttgcactcaaacggagcgtcgagtccgcaacagattgcaatcctgtcttcctcgctacatggttccgtcgcgaatggtccttttggatcgactgcctgtcaaccccaatggtaaagttgatcgacaagagctcacgaggcgcgctcaggatctcccaataagcgagtcatccccagtgcacgtcaaaccgcgtactgaactggaaaggtcgctgtgcgaggagttcgccgatgttataggtttggaagtcggcgttaccgataatttcttcgacctaggcgggcactctctcatggcgatgaaactcgcagctcgcatcagccgtcgttcgaatgcacatatatcagtcaaggacattttcgaccacccgctgattgcagatctcgcaatgaaaattcgg

SEQ ID NO: 8 - protein, 1085 aa, Aureobasidium pullulans
  MISC_FEATURE (1)..(1085): aba1.3 CAT(phe)
EGSKLHTPIPRTAYSGPVEQSFAQGRLWFLDQFNPSSIGYVMPFAARLHGQLQIEALTAALFALEQRHEILRTTLDAHDGVGMQIVHAEHPQQLRIIDVSAKASSSYAQTLRDEQASPFDLSKEPGWRVSLLQLSEIDYVLSIVMHHTIYDGWSLDVLRRELSQFYAAAIRGREPLSTIEPLPIQYRDFSVWQKQEDQVAEHRRQLHYWIEQLDGSSPAEFLNDKPRPTLLSGKAGVVEIAVKGTVYQRLLEFCRLHQVTSFMVLLAAFRATHYRLTGTEDATVGTPIANRNRPELENMIGLFVNTQCIRLKIEDNDTLEELVQHVRATITASISNQDVPFEQVVSALLPGSRDTSRNPLVQLTFAVHSQRNLADIQLENVETNAMPICPSTRFDAEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSIVRLMRHSNVSSRLLLHPRMTHLSNLAFDASVWEIFLTLLNGGTLICIDYLSSLDCRALGVSILEHQVDASVLPPALLKQCLANVPEALASLQVLLSAGDRLDSRDAIESCALVRGSVYNGYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVLIAQTAENEELVGFFTLRQTQAVQSNGAAGVVPEHSDSELAQSCSCTQTERRVRNRLQSCLPRYMVPSRMVLLDRLPVNPNGKVDRQELTRRAQDLPISESSPVHVKPRTELERSLCEEFADVIGLEVGVTDNFFDLGGHSLMAMKLAARISRRSNAHISVKDIFDHPLIADLAMKIR

SEQ ID NO: 9 - DNA, 4467 bp, Aureobasidium pullulans
  misc_feature (1)..(4467): aba1.4 CAMT(phe)
gaaggctccgatctgcacactccaattccccacaggatgtacgttggacctatccagctatcattcgcacagggacgcttgtggttcctcgaccaattgaatttgggcgcatcgtggtacgtcatgccacttgctatgcgcctccaaggctcgctccagctcgacgcgttagagactgcactgtttgctatcgagcagcgacacgaaaccttacggatgacatttgcagaacaagacggagtagctgtacaagtagtgcatgcagcccactacaaacacatcaagatgatcgacaaaccacttagacagaagattgacgtcctgaagatgctggaagaagaacggacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgtagtatcgtccgcttgatgaggcacagcaatgtctcgagtcgccttctgctacatccccgcatgacccacctgtcgaatctcgccttcgatgcgtcggtgtgggagattttcttgacgctgctcaacggtggaacattgatttgtattgactacctctcgtcactagactgtcgtgctcttggggtaagtatcctggaacaccaggttgacgcatcggtacttcctcctgctttgctcaaacaatgcctagcaaatgtccctgaggcacttgcgagcctgcaagtgctcttgtccgctggagatcgactcgacagtcgtgatgctatagagagttgcgcactcgtgcgcggaagtgtctacaatgggtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagcttgccgctcgtatcagccaccgccttcatacacgcatatccgtcaaacacatcttcgatcaccctttgataggcgatttgtctgtccacatagct

SEQ ID NO: 10 - protein, 1489 aa, Aureobasidium pullulans
  MISC_FEATURE (1)..(1489): aba1.4 CAMT(Phe)
EGSDLHTPIPHRMYVGPIQLSFAQGRLWFLDQLNLGASWYVMPLAMRLQGSLQLDALETALFAIEQRHETLRMTFAEQDGVAVQVVHAAHYKHIKMIDKPLRQKIDVLKMLEEERTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSIVRLMRHSNVSSRLLLHPRMTHLSNLAFDASVWEIFLTLLNGGTLICIDYLSSLDCRALGVSILEHQVDASVLPPALLKQCLANVPEALASLQVLLSAGDRLDSRDAIESCALVRGSVYNGYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKLAARISHRLHTRISVKHIFDHPLIGDLSVHIA

SEQ ID NO: 11 - DNA, 3237 bp, Aureobasidium pullulans
  misc_feature (1)..(3237): aba1.5 CAT(pro)
gactctccggtgcctcttttgacaatcacacgtgcccagcacgctggagcagtggagcagtcattcgcacaagctagattgtggttccttgtccagctaggacttgaatctccttcgtacatcataccaattgtattgcgtttacacggttcactctcaaagactgccattgaaggagctctatcagccctgatggaacgtcatgaggtccttcgtacgacgttcgaggaccataagggtatcggcatgcaagtggtacaagaccatcgtcaccaagacttggttgtaattgacgttgcaggtcaggggtcactcgactacaagcagcacttatacatggagcacgtgaaacctttcgatctgacccgggatcctgggtggagggtagcgctgctgcgtctgggagatgacgaccacgtcctctccatcgtaatgcatcacatcatctccgatggctggtcgattgatatcctgctgcgtgagttgggtcagttctactcggccgcgctccgggggcaggacccgttgtcacagacaagtcctctgccgatccagtatcgtgacttcgctctctggcaaaagcaggatcatcaattagccgatcacgagaagcagctgcggtattgggaagagcaactggcggagagctctccagctgagctgctatgtgatcatgcacgtccgacgacgccctcaggtcaggcaggctcgattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggtgcagttgttgttcgctgttcatgcctatcaagaggtcgaaaatttcgccatccccggtgtgcactccgagttggtgcaaggaacgacctttacaagatttgatgtcgagttccacctgcttgaagaccctgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaatgaggcacctgtggtgcagccttctgccacaagccttgcctacgtcatcttcacttccggctcgaccggcaagccgaagggtgtcatggtcgagcaccgcagtatactcagggttgtcacgtctcccccggcccgtgctctgctaccgtccacaatcatcatggcccacctgacaaacattgcattcgatgtatcgctatgggagatatgtacagctcttcttcacggtggtaccctgatctgtattcagtatcttgcctcgctcgatgtcagggggcttcagactacattctctcgcgaagctatcaacgtagctgtgtttcctcctgccttgctaaagacctgtcttgccaagattccatctgctctagcatcgctgagtgccatgttctcgtccggagatcgtctcgactcacgcgatgctagcgagggggccacacttgtgcggcaagggatacacaacgcgtatggtcccacggagaatggcatccagagcacaatctatgaagtcaaagcggacgctgagtttgtcaatggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcgatattgtcccttgattatgtgatcgatgcagccgtccttctgagacagctgattgatcaagagccacaagtggtaggattcgtcattgtatccaccaaacgggcttattcccgacacaacagcggctacgcgtctgaagtttcggcattctgcatcaaagatcagatcgcatggcgcattcgacaacatctctgcaggatgctgccttcctatatggttccctatcaaattgcaattcttgatgaaatgcctatcaatgctaacggcaaggtggatagacagaatcttgcaagcagaactgtcaacgtccaaagaatcctcgccgctccatacatggccccgcgcaatgaagtcgagatttcgctttgcgaacagtatgctgccctgcttgaacacgacgttggcattcttgacgacttcttcgaacttggtggtcactctctcatggctactagactggcctcgcgtatcagctcccgattcagcgctccggtgtctgttcgtgatattttcgaccatccaagaatcatggaccttgctagcatcattcgt

SEQ ID NO: 12 - protein, 1079 aa, Aureobasidium pullulans
  MISC_FEATURE (1)..(1079): aba1.5 CAT(pro)
DSPVPLLTITRAQHAGAVEQSFAQARLWFLVQLGLESPSYIIPIVLRLHGSLSKTAIEGALSALMERHEVLRTTFEDHKGIGMQVVQDHRHQDLVVIDVAGQGSLDYKQHLYMEHVKPFDLTRDPGWRVALLRLGDDDHVLSIVMHHIISDGWSIDILLRELGQFYSAALRGQDPLSQTSPLPIQYRDFALWQKQDHQLADHEKQLRYWEEQLAESSPAELLCDHARPTTPSGQAGSIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLLFAVHAYQEVENFAIPGVHSELVQGTTFTRFDVEFHLLEDPDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRSILRVVTSPPARALLPSTIIMAHLTNIAFDVSLWEICTALLHGGTLICIQYLASLDVRGLQTTFSREAINVAVFPPALLKTCLAKIPSALASLSAMFSSGDRLDSRDASEGATLVRQGIHNAYGPTENGIQSTIYEVKADAEFVNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAILSLDYVIDAAVLLRQLIDQEPQVVGFVIVSTKRAYSRHNSGYASEVSAFCIKDQIAWRIRQHLCRMLPSYMVPYQIAILDEMPINANGKVDRQNLASRTVNVQRILAAPYMAPRNEVEISLCEQYAALLEHDVGILDDFFELGGHSLMATRLASRISSRFSAPVSVRDIFDHPRIMDLASIIR

SEQ ID NO: 13 - DNA, 3252 bp, Aureobasidium pullulans
  misc_feature (1)..(3252): aba1.6 CAT(a-ile)
gctggagacattcaatggtcccggatactgccttctgcttatgaacgtccagtcgagcaatctttcgcacagaatcgcctgtggttcctgtacaagcttgacataggtacgacacagtataatttaccgctggcgatacaccttcgaggaccactagatatatcagcgctgtttatcgcattcaaggcattgactgaaagacatgaacttttgcgcacaacttttgatgaggatgacggaacatgcctgcagatgttattgcctgaatatcagcatgaagtaaggatcaccgacttgcagggatcacacaaaggtagcctcctggatattctcaacaacaatcagaagactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggtcctcgagatctatgcggcccttctgaacggtggtactgtttactgcattgactatctcactacccttgaccctcacgcgcttgagtctgttttcatcgatgctgatctcaacacggcagtccttcctcccgctctacttaaacaggtccttgcttcgagcccttctaccctccatgcccttgatttactcttcataggaggagatcgattggatgctcgtgacgccctgtacgctaatcgtctggttcgagggtcattatacaatgtctatggcccgacagagaacaccgttctgagcgtcgtttacctctttaatgatgacgatgcatgcattaatggcgtccctatcggccaagtcgtcagtaattccggggtatacgtcatggactcagaacagaaattagtacctcctggggtcatgggagaaatcgtcgtgacaggagacggtctcgcaagagggtatactgactcaaccttaaatactgatcgtttcgttcaaatcagtgtcaacggacgtgtactgcaagcataccgtacaggcgatcgtggtcggtaccgcccgacagacgctcgtcttgagttctttggccgtctagatcaacaaatcaagcttcgcgggcatcgtgtagagctcaaagaaatcgagcaagcgatgcttggccacaatgctgttgatgatgcaggagttgtcgctctggagatatctgagtgccaagagctagagatggttggctttgtgactctacgcaatcttggaaccatggaagcaactaacaatctcgcacacacaagctggaacccagtgactctcaaaacccctttagcatcacaaatagtggctgaggttcggggtagactccagcgaaatctgccactctatatggtacccgctacgattgtggtattacatactatgccagtcaatgccaacgggaagctcgaccgacaagcacttgtgaaagctgcaatgacgcttccaaaaactgctccactggtatggatggctccgcgcaatgaaggagagacatcgctatgtgaggagctaacagatatcttgggggtgaacgtcgggatcaccgataacttttttgaccttggggggcattccctcctggcaaccagagtagccgcgcgaatcagccgacgtcttgatgccctggtgaccgtcaaacaaatattcgaccatccagtcattggagatctcgcagctgcaattcaa

SEQ ID NO: 14 - protein, 1084 aa, Aureobasidium pullulans
  MISC_FEATURE (1)..(1084): aba1.6 CAT(a-ile)
AGDIQWSRILPSAYERPVEQSFAQNRLWFLYKLDIGTTQYNLPLAIHLRGPLDISALFIAFKALTERHELLRTTFDEDDGTCLQMLLPEYQHEVRITDLQGSHKGSLLDILNNNQKTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASVLEIYAALLNGGTVYCIDYLTTLDPHALESVFIDADLNTAVLPPALLKQVLASSPSTLHALDLLFIGGDRLDARDALYANRLVRGSLYNVYGPTENTVLSVVYLFNDDDACINGVPIGQVVSNSGVYVMDSEQKLVPPGVMGEIVVTGDGLARGYTDSTLNTDRFVQISVNGRVLQAYRTGDRGRYRPTDARLEFFGRLDQQIKLRGHRVELKEIEQAMLGHNAVDDAGVVALEISECQELEMVGFVTLRNLGTMEATNNLAHTSWNPVTLKTPLASQIVAEVRGRLQRNLPLYMVPATIVVLHTMPVNANGKLDRQALVKAAMTLPKTAPLVWMAPRNEGETSLCEELTDILGVNVGITDNFFDLGGHSLLATRVAARISRRLDALVTVKQIFDHPVIGDLAAAIQ

SEQ ID NO: 15 - DNA, 4467 bp, Aureobasidium pullulans
  misc_feature (1)..(4467): aba1.7 CAMT(val)
gggggttcagtacggcatttaccaataactgcaagcgaggtcgatggacctgttcagcagtccttcgcgcaaaatcgcttgtggttcctagagcagatgaatattggagctacttggtacatcgtaccgttagcagtgcgtctgtacggcacactgcgagttgaggctctgaatattgcgttgcgtacgattcagcaacgccacgaaacattacgaacgaccttcgaagaactaaatgggattgccgttcaacgttgtgattcaacctgccaaggccaattaagggtggtagatttagtcgggcaggggccagatcgctatagagagattctggatgtccagcaaactacaccattcgagctgagccaggagcctggatggagggtagcgctgcttcgtctgggagatgacgaccacgtcctctccatcgtcatgcatcacatcatctccgacggttggtctgtggacgtgctgctacgtgagataggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagttcgccgctcgtatcagccgccggctgaatgctatcgtttcggtcaagaatgtcttcgaccaccccgtacctatggatcttgcagcgacaatccaa

SEQ ID NO: 16 - protein, 1489 aa, Aureobasidium pullulans
  MISC_FEATURE (1)..(1489): aba1.7 CAMT(val)
GGSVRHLPITASEVDGPVQQSFAQNRLWFLEQMNIGATWYIVPLAVRLYGTLRVEALNIALRTIQQRHETLRTTFEELNGIAVQRCDSTCQGQLRVVDLVGQGPDRYREILDVQQTTPFELSQEPGWRVALLRLGDDDHVLSIVMHHIISDGWSVDVLLREIGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKFAARISRRLNAIVSVKNVFDHPVPMDLAATIQ

SEQ ID NO: 17 - DNA, 3258 bp, Aureobasidium pullulans
  misc_feature (1)..(3258): aba1.8 CAT(leu)
gaaggctcaaagcttcatactccaatccctcgcacggcttacagcggtcctgtcgaacagtctttcgcacaaggacgtctttggttccttgaccaattcaatcctagctcgattgggtatgtgatgcctttcgctgcgcgtcttcatggtcaactacaaatcgaagcgctcacagcagcattgttcgctttggaacagcgacatgagatcctgcgaacaacgttggacgcacacgatggtgtaggcatgcagatcgttcacgcggaacatccgcaacagttgagaatcattgatgtgtcagcaaaggcgtcgagcagttatgctcagacactgcgtgacgagcaggcgtcacctttcgacctaagcaaggaaccaggttggagagtctcgttgctgcagctcagtgagatagattatgttctttccattgtaatgcatcacaccatctatgacggttggtctctcgacgtactccggcgggagctaagtcagttttatgccgctgccatccgtggtcgagaacctctatcgacaatcgagccattgcctatccaataccgcgacttttctgtctggcaaaagcaggaagaccaagtcgcagagcatcgacgacagctccattattggatagagcagctagatggcagctctcctgctgagttcctaaacgataaaccacggcctacgttgctttctggcaaggcaggagttgtggaaattgctgtgaagggcactgtatatcaacgtctgctagagttctgcaggcttcatcaggtcacctcgttcatggtgctgcttgcggcattccgagcgacacactatcgtctgacaggcacagaggacgcgactgtcggaacacccatcgccaatcgcaatcgacctgagctggagaacatgattggattgttcgtgaatactcagtgtatacgcctcaagatcgaggacaatgatactctcgaggagctagtacagcacgttcgtgccacgatcacagcatcaatctcgaaccaggatgtaccctttgaacaggtagtgtctgcattgctaccaggatcacgcgacacctctaggaacccactagttcagctgacttttgcggtgcattctcagcgaaatttggctgacattcagctagaaaacgtggagaccaatgctatgccaatttgcccctcgacacgtttcgacgctgaattccacctcttccaagaggagaatatgctaagcggaagggtgctgttttcagacgatcttttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcaccatcatagacgctcgcgcacttggcgttatctttgcgcaacaaagtatcaacgcaaccatgctgtcacctctactcctcaaacaatttttgtcagatgcaccattcgtgctgcgatctctgcatgccctttatctagggggggacagacttcagggtcgtgacgcaatccaggcttgtcgtgtaggttgcgcatttgtcatcaatgcctatggcccaacagagaattctgtcatcagtactacttacacacttgtgaagggaaatgcggacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttctcatagctcaaacagccgaaaatgaagagctagttggcttcttcacgcttcgacaaacccaggctgtgcagtcaaatggtgccgctggtgttgtgccagagcacagcgactccgagctggcgcaatcctgctcttgcactcaaacggagcgtcgagtccgcaacagattgcaatcctgtcttcctcgctacatggttccgtcgcgaatggtccttttggatcgactgcctgtcaaccccaatggtaaagttgatcgacaagagctcacgaggcgcgctcaggatctcccaataagcgagtcatccccagtgcacgtcaaaccgcgtactgaactggaaaggtcgctgtgcgaggagttcgccgatgttataggtttggaagtcggcgttaccgataatttcttcgacctaggcgggcactctctcatggcgatgaaactcgcagctcgcatcagccgtcgttcgaatgcacatatatcagtcaaggacattttcgaccacccgctgattgcagatctcgcaatgaaaattcgg

SEQ ID NO: 18 - protein, 1086 aa, Aureobasidium pullulans
  MISC_FEATURE (1)..(1086): aba1.8 CAT(leu)
EGSKLHTPIPRTAYSGPVEQSFAQGRLWFLDQFNPSSIGYVMPFAARLHGQLQIEALTAALFALEQRHEILRTTLDAHDGVGMQIVHAEHPQQLRIIDVSAKASSSYAQTLRDEQASPFDLSKEPGWRVSLLQLSEIDYVLSIVMHHTIYDGWSLDVLRRELSQFYAAAIRGREPLSTIEPLPIQYRDFSVWQKQEDQVAEHRRQLHYWIEQLDGSSPAEFLNDKPRPTLLSGKAGVVEIAVKGTVYQRLLEFCRLHQVTSFMVLLAAFRATHYRLTGTEDATVGTPIANRNRPELENMIGLFVNTQCIRLKIEDNDTLEELVQHVRATITASISNQDVPFEQVVSALLPGSRDTSRNPLVQLTFAVHSQRNLADIQLENVETNAMPICPSTRFDAEFHLFQEENMLSGRVLFSDDLFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTIIDARALGVIFAQQSINATMLSPLLLKQFLSDAPFVLRSLHALYLGGDRLQGRDAIQACRVGCAFVINAYGPTENSVISTTYTLVKGNADFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVLIAQTAENEELVGFFTLRQTQAVQSNGAAGVVPEHSDSELAQSCSCTQTERRVRNRLQSCLPRYMVPSRMVLLDRLPVNPNGKVDRQELTRRAQDLPISESSPVHVKPRTELERSLCEEFADVIGLEVGVTDNFFDLGGHSLMAMKLAARISRRSNAHISVKDIFDHPLIADLAMKIR

SEQ ID NO: 19 - DNA, 4467 bp, Aureobasidium pullulans
  misc_feature (1)..(4467): aba1.9 CAMT(val)
gaaggctccgatctgcacactccaattccccacaggatgtacgttggacctatccagctatcattcgcacagggacgcttgtggttcctcgaccaattgaatttgggcgcatcgtggtacgtcatgccacttgctatgcgcctccaaggctcgctccagctcgacgcgttagagactgcactgtttgctatcgagcagcgacacgaaaccttacggatgacatttgcagaacaagacggagtagctgtacaagtagtgcatgcagcccactacaaacacatcaagatgatcgacaaaccacttagacagaagattgacgtcctgaagatgctggaagaagaacggacgactcccttcgagctgagccgcgagcctggatggagggtagcgctgctgcgtctgggagatgacgaccacgtcctctccatcgtcatgcatcacatcatctccgacggttggtctgtggacgtgctgcgccacgagctaggtcagttctactcggccgcgctccgggggcaggacccgttgtcgcagataagtcctctgccgatccagtatcgtgacttcgctctctggcagagacaagacgagcaagttgcggagcatcagcgccagctggagcattggacagagcagttggcagacagttcacccgccgagttgttgagcgaccacccgaggccatcgattctttctggccaggcgggcgctattcccgtcaatgttcaaggctctctgtatcaggcgcttcgggcgttctgccgcgctcaccaggtcacctctttcgtagtcctgctcacggcgttccgcatagcacactatcgtctgacgggtgcggaggacgcaaccattggaactcccattgcaaatcgcaaccggccagagctcgagaacatgatcggtttcttcgtcaatacacaatgcatgcgcatcgtcattggcagtgacgacacatttgaagggctggtgcagcaagtacgctcgataactgcagctgcccacgagaaccaggacgttccattcgagcgcatcgtgtcagcactgcttcccggttctagagacacatcacgcaatcctctggttcagctcatgtttgctgtccactcgcaaagaaaccttggtcagatcagtctagaaggcctgcagggtgaattgctgggagtggcagcgactacgagattcgatgtagagttccatctcttccaagatgacgacaagctcagcggcaacgtgctcttcgcgaccgagctcttcgagcagaagactatgcaaggcatggtcgacgtgttccaggaagtgctcagccggggccttgagcagccccagatacctctggcgaccctcccgctcacgcacggactggaggagctcaggaccatgggtcttctcgacgtggagaagacagactaccctcgagagtcgagcgtggtggacgtgttccgtgagcaagcggctgcctgctccgaggcgattgcggtcaaagactcgtcggcgcagctcacctactcggagctcgatcgacagtcggacgagcttgccggctggctgcgccagcaacgtcttcctgcggagtcgttggttgcagtgctggcacccaggtcgtgccagaccattgtcgcgttcctgggcatcctcaaggcgaatctggcatacctgccgctagacgtcaacgtgcccgctactcgcctcgagtcgatactgtctgccgtcggcggccggaagctggtcttgcttggagctgacgtggccgaccctggccttcgcctggcggatgtggagctcgtgcggatcggcgacacactcggccgctgtgtacccggggcgcccggcgacaacgaggcacctgtggtgcagccttctgccacaagccttgcctacgtcatcttcacttccggctcgaccggcaagccgaagggtgtcatggtcgagcaccggggtgtagtgcgacttgtcaagcagagcaatgttgtctaccatctcccgtccacatctcgcgtggcccacctgtcgaatctcgcctttgatgcctcggcgtgggagatctatgcggcactgcttaatggcggtacactcatctgcattgactatttcacaactctagactgctctgctctcggcgccaaattcatcaaggagaagatcgtcgcgaccatgattccgccagcgcttctgaagcaatgtctggcgatcttcccgaccgctcttagtgaactggtcctgctgtttgctgccggagatcgattcagcagtggcgatgccgtcgaagtgcagcgccacaccaaaggcgctgtttgtaacgcgtacggaccgacagaaaacaccattcttagtacgatctacgaagtcaagcagaatgagaacttcccgaacggtgtgcctatcggccgcgctgtgagcaactcaggggcatatgtcatggacccgcagcagcaactggtgcctctcggggtgatgggcgagctcgtcgtcaccggcgacggcctggcccgtggttacaccgacccgtcactggatgcggaccgctttgtgcaggtctccgtcaacgggcagctcgtgagagcgtaccgaacaggcgatcgcgtgcgctgcaggccttgcgatggccagatcgagttctttggacgtatggaccggcaagtcaagatccgaggacatcgcatcgagctcgcagaggtagagcatgcggtgcttggcttggaagacgtgcaagacgctgccgttatcgcatttgacaatgtggacagcgaagagccagaaatggttgggtttgtcactattaccgaagacaatcctgtccgtgaggacgaaaccagcggtcaagtagaagactgggcgaaccacttcgagataagtacctacaccgatatcgcggcgatcgatcagggtagcattggaagtgactttgtaggttggacttctatgtacgacggaagcgagatcgacaaggcagagatgcaagaatggcttgccgataccatggcctctatgctcgacgggcaggcgccgggcaatgtgttagagataggtacaggcactggcatggtcctcttcaatctcggcgacggactgcagagctatgtcggcctcgaaccatcaagatcggcggccgcttttgtcaaccagacgattaagtcgctccccacccttgctggcaacgctgaagtacacattggcactgcgaccgacgtggcccgtctagatggcctccgccccgacttagtggtagtcaattcggtagtccagtacttcccatcaccagagtacctaatggaagtcgtggaggctcttgcacgtctgccgggcgtcgagcgaattttcttcggagacgtacgttcgtacgccatcaacagagatttcctggctgccagagctctacacgaacttggcgacagagcgactaagcacgagattcggcgaaagatgctagagatggaagaacgcgaagaggagctgctcgtcgacccagctttcttcaccatgttgaccagcagtctccctggcctgattcagcatgtcgagatcttgccgaagctgatgagagccactaatgagctcagcgcgtatcgatacactgctgtagtacacgtgtgccgtgccggtcaagagcctcgttccgtgcatacgatcgacgacgatgcctgggtgaatcttggagcttctcggttgagtcgccctaccctttcaagccttttgcaaacttccgagggcgcatcggccgtcgcagtaagcaatattccttacagcaagaccatcacagagcgagcgctcgttagtgcgctcgatgaggatgatatgcaagactcatcggactggctgctggccgtgcgcgagacaggcagatcttgttcctccttctccgcaacagaccttgtcgagcttgctcgagagacgggctggcgtgtggagctcagctgggcacgacagtactcacagaaaggcgcactcgatgctgtcttccacagacaccctgtttccgctgggagcgggcgtgtcatgttccagtttccagttgagaccgaagatcgaccgcacatctcacgcacgaaccgacctttacagcgattgcagaagaagcgaaccgagacacatgttcatgagcagttgcgggctttgcttccacgatacatggttcctacgcggattgtggcgcttgataagctgcccgtcaatgcaaacggcaaggttgatcgtcaacagctcgctaggacagcccaggttctcccagcgagcaaggcgccgtctgcatgcgtggccccacgcaacgaattggaaatgacactgtgtgaagagttctcgcaggttcttggcgtcgaggtcggcattactgacaatttcttccacctgggtggccactctctcatggcaacaaagcttgccgctcgtatcagccgtcaactaaatatccaagtctcagtccgagacatctttgactatcccgttatagtcgacctcacagacagattgaga

SEQ ID NO: 20 - protein, 1489 aa, Aureobasidium pullulans
  MISC_FEATURE (1)..(1489): aba1.9 CAMT(val)
EGSDLHTPIPHRMYVGPIQLSFAQGRLWFLDQLNLGASWYVMPLAMRLQGSLQLDALETALFAIEQRHETLRMTFAEQDGVAVQVVHAAHYKHIKMIDKPLRQKIDVLKMLEEERTTPFELSREPGWRVALLRLGDDDHVLSIVMHHIISDGWSVDVLRHELGQFYSAALRGQDPLSQISPLPIQYRDFALWQRQDEQVAEHQRQLEHWTEQLADSSPAELLSDHPRPSILSGQAGAIPVNVQGSLYQALRAFCRAHQVTSFVVLLTAFRIAHYRLTGAEDATIGTPIANRNRPELENMIGFFVNTQCMRIVIGSDDTFEGLVQQVRSITAAAHENQDVPFERIVSALLPGSRDTSRNPLVQLMFAVHSQRNLGQISLEGLQGELLGVAATTRFDVEFHLFQDDDKLSGNVLFATELFEQKTMQGMVDVFQEVLSRGLEQPQIPLATLPLTHGLEELRTMGLLDVEKTDYPRESSVVDVFREQAAACSEAIAVKDSSAQLTYSELDRQSDELAGWLRQQRLPAESLVAVLAPRSCQTIVAFLGILKANLAYLPLDVNVPATRLESILSAVGGRKLVLLGADVADPGLRLADVELVRIGDTLGRCVPGAPGDNEAPVVQPSATSLAYVIFTSGSTGKPKGVMVEHRGVVRLVKQSNVVYHLPSTSRVAHLSNLAFDASAWEIYAALLNGGTLICIDYFTTLDCSALGAKFIKEKIVATMIPPALLKQCLAIFPTALSELVLLFAAGDRFSSGDAVEVQRHTKGAVCNAYGPTENTILSTIYEVKQNENFPNGVPIGRAVSNSGAYVMDPQQQLVPLGVMGELVVTGDGLARGYTDPSLDADRFVQVSVNGQLVRAYRTGDRVRCRPCDGQIEFFGRMDRQVKIRGHRIELAEVEHAVLGLEDVQDAAVIAFDNVDSEEPEMVGFVTITEDNPVREDETSGQVEDWANHFEISTYTDIAAIDQGSIGSDFVGWTSMYDGSEIDKAEMQEWLADTMASMLDGQAPGNVLEIGTGTGMVLFNLGDGLQSYVGLEPSRSAAAFVNQTIKSLPTLAGNAEVHIGTATDVARLDGLRPDLVVVNSVVQYFPSPEYLMEVVEALARLPGVERIFFGDVRSYAINRDFLAARALHELGDRATKHEIRRKMLEMEEREEELLVDPAFFTMLTSSLPGLIQHVEILPKLMRATNELSAYRYTAVVHVCRAGQEPRSVHTIDDDAWVNLGASRLSRPTLSSLLQTSEGASAVAVSNIPYSKTITERALVSALDEDDMQDSSDWLLAVRETGRSCSSFSATDLVELARETGWRVELSWARQYSQKGALDAVFHRHPVSAGSGRVMFQFPVETEDRPHISRTNRPLQRLQKKRTETHVHEQLRALLPRYMVPTRIVALDKLPVNANGKVDRQQLARTAQVLPASKAPSACVAPRNELEMTLCEEFSQVLGVEVGITDNFFHLGGHSLMATKLAARISRQLNIQVSVRDIFDYPVIVDLTDRLR

SEQ ID NO: 21 - DNA, 1431 bp, Aureobasidium pullulans
  misc_feature (1)..(1431): aba1 c-term condensation
ctccatcatacgcgtatccttactcatgatcatggacaacatggacagccagacctcaagccattcaccttgctaccaaccaacaatcctcaagaattcctacagcatcacattttgccacaacttgttcccgatcatgcgaagatcctcgatgtgtatcccgttacaagaatacagagaaggtttcttcatcatccgaagcgcggcctccctcgttttccctccatggtcttctttgacttccctcctggttcagacccacacaagctaagattagcttgtatggcattagtccagcgtttcgacattcttcgcacaatcttcctttctgtttcgggtcaattcttccaagtggtcctggatggatatgggattgtcataccggtcatcgaggttgacgaagagctagacgacgccacccgtaaattacacgattccgatattcagcagcccttacggttgggaaaaccgttaatacgcattgctgtcttgaaaaggcagcactccagagtacgagcagtcttgcgcttgtcgcatgctctctatgatggtttgagctttgagcatatcatccaatctcttcatgccctttatctcgatatcaccctttcggccccaccgaagtttggactctacgtacaacatatgatacaaagtcgcgcagaaggttatgctttctggcggtctgtcttgaagggctcgtcgatgacaattctcgagcgttctagcacccttcaatcgcggcagccgcatcttggacgttttctctctgcggagaaaattattaaggctcctttacacgccaacaagtctggaatcacacaggcaacagtgttcgcggccgcaaacgcactcatgcttgcgaatcttactggtactaatgacgttgtgtttgcccgcattgtctctggacgtcaatctttgcctaagaactttcagcacgttgtgggaccttgcacgaacgatgtgcccgttcgcgtacgcatggagcctggcgtgggaccaaaagctttactcagacaggtgcaagaccagtatgttcatagcttccctttcgaaacactaggattcgacgagatcaaggagaactgtacggactggccagaaagaatcacgaattttgggtgttctacaacttaccagaactttgacatttttcccaaaagtcagattgaccaccagcagattcaaatggctagcttggcaagcgagtatcagaatcgagaaacctgggacgaagcgccgctatacgacctcaatgtcacaggagtacctcagcctgacggacgtcatatcaagatatacgtgggtgtagacgggcagctttgcgatgaaagcacgcttgattgcattctctcggatatttgtgagggtgtggtctcgctcacagacgctttgcaagaacttcccgctgctagcattactgagtag

SEQ ID NO: 22 - protein, 476 aa, Aureobasidium pullulans
  MISC_FEATURE (1)..(476): aba1 c-term condensation
LHHTRILTHDHGQHGQPDLKPFTLLPTNNPQEFLQHHILPQLVPDHAKILDVYPVTRIQRRFLHHPKRGLPRFPSMVFFDFPPGSDPHKLRLACMALVQRFDILRTIFLSVSGQFFQVVLDGYGIVIPVIEVDEELDDATRKLHDSDIQQPLRLGKPLIRIAVLKRQHSRVRAVLRLSHALYDGLSFEHIIQSLHALYLDITLSAPPKFGLYVQHMIQSRAEGYAFWRSVLKGSSMTILERSSTLQSRQPHLGRFLSAEKIIKAPLHANKSGITQATVFAAANALMLANLTGTNDVVFARIVSGRQSLPKNFQHVVGPCTNDVPVRVRMEPGVGPKALLRQVQDQYVHSFPFETLGFDEIKENCTDWPERITNFGCSTTYQNFDIFPKSQIDHQQIQMASLASEYQNRETWDEAPLYDLNVTGVPQPDGRHIKIYVGVDGQLCDESTLDCILSDICEGVVSLTDALQELPAASITE

SEQ ID NO: 23 - DNA, 1700 bp, Aureobasidium pullulans
  misc_feature (1)..(1700): 5′ regulatory region of the aba1 gene
gagaaacgatacattgtaatacttagcgaactccttgcgatactgagatccctcggcatgaaacagtcccgcttgttattggacgtgctttacatggcagttactataggaatatctgcttctacgcgcaagactactacctgagcctgtgtatatgggacgtttctccagcgttatgtgactcagccccaaacttcagagcaacacatcgtttcgtattgagaatataaagaatgacgaagcaggggatttgcaactagcaggcgtgaaagactatgcaagtctctgtttgttgagatattgtggactgaggatttggcaatacttgacggaggaggcaccctgctccgcactcgccatgaattcaaagccgttggagtccctgcaagcaagatttctgattggctgaatatcatttcggggacctaaagcttcgcagagaatgacgaggcagcttcacgcgtctttcgatgcgcctcaaccgcttccctttgatcatcagccatcaatcgttctgattaccactctcttgaccacttcttgctgaatgtggaagctgaacctgccactctcctcctcttacaatgattacaacaagaatctcttggatctaagtatcgcgtcatgtgttttatagctaatccagggcatagtttctaggttccctgaggcaaccaacttcttacctctcgaagttcagtcattgcgcatgtgtgaagatcggtggctgaaccaacgcagcgactcgattaccccttcctctcccagatgaccatgaatttcctctcgatcaatctaatgtctgttgattgtagaaggggaattggtagctatagaaggacagtgtcgagggtccgagccagtctttctgtttttgattttgattacaatgagtgcctcaagaactggcagaagaagctccgctccgagaggcttggtctaccaatcgacgaaatacagtaggatcttgtgctacattggattgctcagagagacagggtcgtggtgacagtgacgattttgtgtcggccttggcatcagcattcccctttgtttaatcgatatggagcagactgttagctagagtagtggcaacgcatggtaacgtgtgtcagggtgttttgaagcaaagaaggtattgagtcgcggatttctaccgaggtgacggaaatctgccaagcctcgagccgagagaaaacatcctggtcgacgttctggactttgaagtgtcacagacttttcccaactccgctctgcttcgcgtcaggcaagtgcatcaccgaagtcggcggttggtgaacttggcgttcggagtggcatcttataacgatggcaactgttgccaacaaagcggaaaatttctctatgcggaggtgtgttacgaatctgcctaacttttgcctcatccactgacgactgactaatcacagggaggttagtgttcgctgccgagggcgccatgtcaggcgtcgcccggcagagttgatcatgcaaattcgaagtgagcaccgatcagatgagtcaagtcaatatgcattgtcttatagccagacgatatgcgcgccacaacgattcttgctgacatcacccatatacagtcataagtgtagttcgcttacggagctcacatgaccgcaatgtctatcagagtaagcatgtctgtcgtggctgacgctgctctaacaatgatgcgtgaagca

SEQ ID NO: 24 - DNA, 22 bp, Artificial
  synthetically generated oligonucleotide
ccggcaccaccggnaarcchaa

SEQ ID NO: 25 - DNA, 25 bp, Artificial
  synthetically generated oligonucleotide
tcacctccggcaccachggnaarcc

SEQ ID NO: 26 - DNA, 29 bp, Artificial
  synthetically generated oligonucleotide
gtccacggacggatgtacarrachggvga

SEQ ID NO: 27 - DNA, 24 bp, Artificial
  synthetically generated oligonucleotide
ccggaccatgtcgccngtbykrta

SEQ ID NO: 28 - DNA, 28 bp, Artificial
  synthetically generated oligonucleotide
gctgcatggcggtgatgswrtsnccbcc

SEQ ID NO: 29 - DNA, 25 bp, Artificial
  synthetically generated oligonucleotide
agccttctgccacaagccttgccta

SEQ ID NO: 30 - DNA, 25 bp, Artificial
  synthetically generated oligonucleotide
agcatcgcgtgagtcgagacgatct

SEQ ID NO: 31 - DNA, 20 bp, Artificial
  synthetically generated oligonucleotide
accgctttgtgcaggtctcc

SEQ ID NO: 32 - DNA, 23 bp, Artificial
  synthetically generated oligonucleotide
caagtgtgtaagtagtactgatg

SEQ ID NO: 33 - DNA, 20 bp, Artificial
  synthetically generated oligonucleotide
aatctatgaagtcaaagcgg

SEQ ID NO: 34 - DNA, 21 bp, Artificial
  synthetically generated oligonucleotide
ccgctttgacttcatagattg

SEQ ID NO: 35 - DNA, 21 bp, Artificial
  synthetically generated oligonucleotide
tcagtactacttacacacttg

SEQ ID NO: 36 - DNA, 21 bp, Artificial
  synthetically generated oligonucleotide
aacgtgctcttcgcgaccgag

SEQ ID NO: 37 - DNA, 25 bp, Artificial
  synthetically generated oligonucleotide
tcgcgtatcagctcccgattcagcg

SEQ ID NO: 38 - DNA, 22 bp, Artificial
  synthetically generated oligonucleotide
cgtcttgtctctgccagagagc

SEQ ID NO: 39 - DNA, 20 bp, Artificial
  synthetically generated oligonucleotide
tggatcgaaagcgcgagctg

SEQ ID NO: 40 - DNA, 20 bp, Artificial
  synthetically generated oligonucleotide
tgttctccaagtcgagaatg

SEQ ID NO: 41 - DNA, 19 bp, Artificial
  synthetically generated oligonucleotide
atccaggccgatcgcgctg

SEQ ID NO: 42 - DNA, 22 bp, Artificial
  synthetically generated oligonucleotide
agaatcgcacaatatcctccag

SEQ ID NO: 43 - DNA, 26 bp, Artificial
  synthetically generated oligonucleotide
tttttttttttttttttttttttttv

SEQ ID NO: 44 - DNA, 43 bp, Artificial
  synthetically generated oligonucleotide
ctactactactaggccacgcgtcgactagtacgggnngggnng

SEQ ID NO: 45 - DNA, 36 bp, Artificial
  synthetically generated oligonucleotide
ggccacgcgtcgactagtacgggnngggnngggnng

(19) United States
(12) Patent Application Publication
(10) Pub. No.: US 2009/0325155 A1
(43) Pub. Date: Dec. 31, 2009

(54) Aureobasin a synthetase

(76) Inventors: Ake P. Elhammer, Kalamazoo, MI (US); Jerry L. Slightom, Pontage, MI (US); Brian P. Metzger, Portage, MI (US)

Correspondence Address: HONIGMAN MILLER SCHWARTZ & COHN LLP, 444 WEST MICHIGAN AVENUE, KALAMAZOO, MI, 49007-3714, US

(21) Appl. No.: 11/991,040

(22) PCT Filed: Aug. 25, 2006
(86) PCT No.: PCT/US06/33308

§ 371 Date: May 12, 2009

Related U.S. Application Data
(60) Provisional application No. 60/711,529, filed on Aug. 26, 2005.
(60) Provisional application No. 60/732,578, filed on Nov. 2, 2005.

(51) Int. Cl. C12Q 1/68; C12N 15/11; C12N 15/00; C12N 1/15; C12P 21/02; C12N 9/00

(52) U.S. Cl. 435/6; 536/23.2; 435/320.1; 435/254.11; 435/69.1; 435/183

(57) ABSTRACT

Disclosed are polynucleotides encoding polypeptides having Aureobasidin A synthetase activity and Aureobasidin A synthetase-like activity. The invention also provides methods for detecting AbA synthetase proteins and nucleic acids and AbA synthetase-like proteins and nucleic acids in cells, and method for producing AbA synthetase polypeptides.

This application claims the benefit of the U.S. Provisional application No. 60/711,529, filed on Aug. 26, 2005 and the U.S. Provisional application No. 60/732,578, filed on Nov. 2, 2005, both of which are incorporated herein by reference.

### BRIEF DESCRIPTION OF THE INVENTION

The invention relates to nucleotide sequences and polypeptides encoded by the nucleotide sequences which possess Aureobasidin A synthetase-like activity.

### BACKGROUND OF THE INVENTION

Aureobasidin A (AbA) is a cyclic depsipeptide (see figure below), including one hydroxy acid and eight amino acids, with a molecular weight of about 1,100 Daltons. AbA is an antibiotic that is toxic at a low concentration (0.1-0.5 μg/ml) against a number of fungi, including yeasts, such as Saccharomyces cerevisiae and Schizosaccharomyces pombe. More importantly, AbA is cidal to several fungal pathogens, including the two major pathogens Candida spp and Cryptococcus neoformans. Hence, the compound has significant potential for the development of a novel pharmaceutic(s). It is, however, not toxic to the third major human pathogen, Aspergillus spp. Until now this has hampered its development into a marketed product. On the other hand, synthetic chemistry-based, exploratory work on AbA has demonstrated that certain structural modifications can convert the native molecule into compounds that have close to equal efficacy towards Candida spp., C. neoformans and Aspergillus spp. (summarized in Kurome and Takesako, 2000).

Cyclic peptides are produced by microorganisms such as bacteria and fimgi. AbA is produced by the fungus, Aureobasidium pullulans R-106 (also referred to as BP-1938; Takesako et al., 1993). AbA comprises 8 amino acids and one hydroxy acid, arranged in the sequence: (2R,3R)-hydroxy-methylpentanoic acid, L-N-methyl valine, L-phenylalanine, L-N-methyl phenylalanine, L-proline, L-allo-isoleucine, L-Leucine, L-N-methyl valine and L-hydroxymethyl valine. Hence AbA contains four N-methylated amino acids, two non-proteinogenic amino acids and one D-configured hydroxyacid. These characteristics strongly suggest that the molecule is generated by a very specific type of enzymatic system, referred to as a Non-Ribosomal Peptide Synthetase (NRPS) complex, in the producer organism.

Native AbA has the following structure:

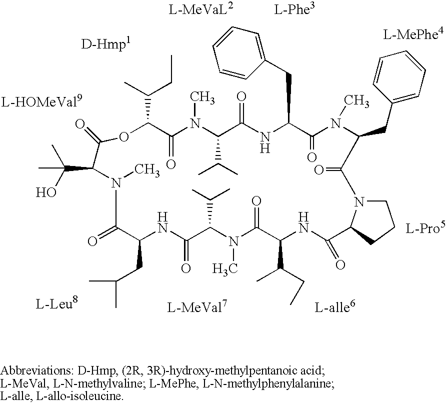

NRPS complexes are large enzyme complexes composed of an assembly line-like arrangement of biosynthetic modules, each of which is responsible for insertion, and in some cases modification, of an amino acid (or other biosynthetic unit), into the sequence of the final cyclized peptide product. (reviewed by Marahiel et al., 1997). The biosynthetic modules (in a NRPS complex) are, in turn, typically composed of several domains, each of which has a specific function in the assembly of the polypeptide. Since the amino acid recruiting domains in the biosynthetic modules each are specific for a certain amino acid, the sequential arrangement of the modules in the complex, in itself, determines the sequence and structure of the cyclic peptide produced. From this it also follows that the number of biosynthetic modules in a NRPS complex coincides with the number of amino (or hydroxyl) acids in the sequence of the peptide produced by the complex (Marahiel et al., 1997). For instance, the ACV synthetase, which produces a three amino acid peptide (aminoadipic acid, cysteine and valine; Smith et al., 1990, MacCabe et al., 1991, Gutierrez et al., 1991) comprises three modules, and tyrocidine synthetase, which is responsible for biosynthesis of the 10 amino acid antibiotic Tyrocidine A, is composed of ten modules (Weckermann et al., 1988; Turgay et al., 1992; Mootz and Marahiel, 1997).

Fungal NRPS complexes typically comprise a single, very large polypeptide. For instance, the cyclosporine NRPS complex in Tolypocladium niveum, which is responsible for the biosynthesis of the immunomodulatory compound Cyclosporin A, is a 1.6 million Dalton protein (Weber et al. 1994). Fungal NRPS proteins also include a specialized condensation domain rather than the thioesterase domain commonly found in bacterial NRPS complexes that may catalyze the final cleavage and cyclization of the peptide product (see below).

The NRPS catalyzed biosynthesis of cyclic peptides proceeds by a thiotemplate process. Each amino acid in the sequence is activated in the form of an adenylate, then bound to the NRPS complex in the form of a thioester and then linked with the following amino acid in the peptide. Hence, the cyclic peptide is assembled step-wise as a linear precursor on the NRPS complex. The amino acid recruiting Adenylation (A) domains in the complex modules, each of which are specific for a particular amino acid, are responsible for recruiting the appropriate amino (or hydroxy) acid for the sequence in the peptide. The recruited amino acids are linked to thiolation (T) domains which anchor the nascent peptide, via a thioester linkage, to the NRPS complex during peptide assembly. (See above.) These domains are also believed to be important for presenting the amino acids in a position conducive to efficient peptide bond formation. Condensation (C) domains catalyze condensation of the amino group of one amino acid to the carboxyl group of an adjacent amino acid, forming the peptide bonds in the sequence. Methylation (M) domains catalyze N-methylation (if present) of adjacent amino acids. And epimerization (E) domains may catalyze the conversion of L-amino acids to D-amino acids (if present). Alternatively, some fungi may instead use (a) D-amino acid-specific adenylation domain(s) for introduction of D-amino acids. Finally, a thioesterase (Te) domain or, in fungi, a specialized condensation domain catalyzes the release of the precursor by cleavage of the linkage to a complex thiolation domain, as well as the final cyclization of the peptide. The overall mechanism readily explains the specific characteristics associated with many cyclic peptides, such as the presence of non-proteinogenic amino acids, N-methylated amino acids, D-amino acids, ester bonds, and also the final cyclization of the molecules.

Since each domain in a NRPS complex is specific for a certain amino acid (or modification), the sequential arrangement of the domains in the complex does, in itself, determine the sequence and structure of the cyclic peptide produced.

The linear, assembly-line-like arrangement of the NRPS complex proteins are the products of a similar linear arrangement of the corresponding gene sequences. The complete sequence of the corresponding NRPS gene will provide information regarding the modular organization of the gene.

Neither the DNA sequence encoding the AbA NRP synthetase (ABA) nor the amino acid sequence of the enzymatic complex is known.

### SUMMARY OF THE INVENTION

The invention provides polypeptides and polynucleotides that encode an enzyme possessing AbA NRP synthetase-like activity. The invention also provides methods for detecting AbA NRP synthetase-like proteins and nucleic acids in cells, and methods for producing AbA NRP synthetase polypeptides.

In a first aspect, the invention provides an isolated polynucleotide encoding an amino acid sequence as set forth in SEQ ID NO:2. The isolated polynucleotide can be SEQ ID NO:1, SEQ ID NO:1 where T can also be U, a nucleic acid sequence complementary to SEQ ID NO:1, and fragments of SEQ ID NO:1 that are at least 20 (at least 25, 24, 23, 22, or 20) bases in length and that hybridize under stringent conditions to DNA that encodes the polypeptide of SEQ ID NO:2 or encodes a polypeptide that has Aureobasidin A synthetase activity.

In an embodiment of the first aspect, the isolated nucleic acid comprises a sequence at least 95% identical to SEQ ID NO: 1 that encodes a polypeptide that has Aureobasidin A synthetase activity or that catalyzes the synthesis of Aureobasidin A and related molecules.

In another embodiment, the isolated nucleic acid comprises a sequence that encodes a polypeptide at least 95% identical to SEQ ID NO:2, or encodes a polypeptide with up to 1100 (up to 1100, 1000, 900, 800, 700, 500, 500, 400, 300, 200, 100, or 50) conservative amino acid substitutions, deletions or insertions wherein the polypeptide has Aureobasidin A synthetase activity or catalyzes the synthesis of Aureobasidin A and related molecules. The isolated nucleic acid can also comprise a sequence that encodes an immunogenic fragment of SEQ ID NO:2 at least 7 (at least 50, 40, 30, 20, 15, 12, 10, 9, 8 or 7) residues in length.

In a second aspect, the invention provides an isolated nucleic acid that comprises SEQ ID NO:23 or a fragment of SEQ ID NO:23 that hybridizes under stringent conditions to a hybridization probe at least 20 (at least 25, 24, 23, 22, 21 or 20) nucleotides in length. In an embodiment of the second aspect, the isolated nucleic acid can be operably linked to a heterologous coding sequence or to SEQ ID NO: 1, or fragments thereof.

In a third aspect, the invention provides nucleic acids that encode modules of Aureobasidin A synthetase. The nucleic acids comprise a sequence that hybridizes under stringent conditions to a probe of at least 20 (at least 25, 24, 23, 22, 21, or 20) bases in length, wherein the sequence is selected from the group consisting of SEQ ID NOs 3, 5, 7, 9, 11, 13, 15, 17, 19, and 21.

In an embodiment of the third aspect, the hybridization probe encodes a biosynthetic module of Aureobasidin A synthetase. In another embodiment, the nucleic acid comprises a sequence at least 95% identical to a sequence selected from the group consisting of SEQ ID NOs 3, 5, 7, 9, 11, 13, 15, 17, 19, and 21. In another embodiment, the nucleic acid encodes a polypeptide with up to 150 (up to 150, 140, 130, 120, 110, 100, 90, 80, 70, 60, 50, 40, 30, 20, 10, or 5) amino acid substitutions, deletions or insertions, wherein the polypeptide sequence is selected from the group consisting of SEQ ID NOs 6, 8, 10, 12, 14, 16, 18, and 20. In yet another embodiment, the nucleic acid comprises a sequence that encodes an immunogenic fragment of a polypeptide at least 7 (at least 7, 8, 9, 10, 12, 15, 18, or 20) amino acid residues in length, the sequence of which is selected from the group consisting of SEQ ID NOs 4, 6, 8, 10, 12, 14, 16, 18, 20, and 22. In a further embodiment, the nucleic acid encodes a polypeptide with up to 85 (up to 80, 70, 60, 50, 40, 30, 20, 10, or 5) amino acid substitutions, deletions or insertions, wherein the polypeptide is SEQ ID NO:4. In an additional embodiment, the nucleic acid encodes a polypeptide with up to 45 (up to 40, 30, 20, 10, 5, or 3) amino acid substitutions, deletions or insertions, wherein the polypeptide sequence is SEQ ID NO:22.

The nucleic acid molecules of the invention are not limited strictly to molecules including the sequences set forth as SEQ ID NOs: 1, 3, 5, 7, 9, 11, 13, 15, 17, 19, 21, or 23. Rather, the invention encompasses nucleic acid molecules carrying modifications such as substitutions, small deletions, insertions, or inversions, which nevertheless encode proteins having substantially the biochemical activity of ABA according to the invention, and/or which can serve as hybridization probes for identifying a nucleic acid with one of the disclosed sequences. Included in the invention are nucleic acid molecules, the nucleotide sequence of which is at least 95% identical (e.g., at least 96%, 97%, 98%, or 99% identical) to the nucleotide sequences shown as SEQ ID NO: 1, 3, 5, 7, 9, 11, 13, 15, 17, 19 or 21 in the Sequence Listing. The invention also includes nucleic acid molecules, the nucleic acid sequence of which is at least 70% identical (70, 75, 80, 85, 90, and 95% identical) to the nucleotide sequences shown as SEQ ID NO: 1, 3, 5, 7, 9, 11, 13, 15, 17, 19 or 21 in the Sequence Listing.

In a fourth aspect, the invention provides vectors comprising nucleic acids of SEQ ID NOs 1, 3, 5, 7, 9, 11, 13, 15, 17, 19, 21 and 23 or nucleic acids that encode Aureobasidin A synthetase or similar polypeptides or fragments thereof. In one embodiment, the vector is an expression vector, wherein the nucleic acid is operably linked to an expression control sequence. In another embodiment, a cell comprises the vector. The cell can be transfected with one or more of the vectors or can be a progeny of the cell. In another embodiment, the transfected cell or a progeny thereof, expresses a polypeptide having Aureobasidin A synthetase activity, or a fragment of the polypeptide.

The invention also, in a fifth aspect; provides a method for producing Aureobasidin A synthetase or related polypeptides and for producing Aureobasidin A and related molecules. The method includes transforming a host cell with an expression vector containing an Aureobasidin A synthetase polynucleotide, expressing the polynucleotide in the host, and recovering the Aureobasidin A synthetase polypeptide. The method also includes recovering Aureobasidin A or Aureobasidin A-like molecules.

In a sixth aspect the invention provides nucleic acids that interact with Aureobasidin A synthetase polynucleotides. In one embodiment, the nucleic acid is a single stranded nucleic acid that hybridizes to a probe having a sequence selected from the group consisting of SEQ ID NOs, 3, 5, 7, 9, 11, 13, 15, 17, 19, 21, and 23. In another embodiment the nucleic acid comprises at least 10 (at least 12, 15, 20 or 25) consecutive nucleotides of the complement of the sequence selected from the group consisting of SEQ ID NOs 1, 3, 5, 7, 9, 11, 13, 15, 17, 19, 21, and 23. In yet another embodiment, the nucleic acid is an antisense oligonucleotide that inhibits the expression of Aureobasidin A synthetase. In still another embodiment, a method of hybridization includes contacting an antisense oligonucleotide with a nucleic acid selected from the group consisting of SEQ ID NOs 1, 3, 5, 7, 9, 11, 13, 15, 17, 19, 21, and 23.

In a further embodiment, the invention provides a double-stranded ribonucleic acid (dsRNA) comprising a first strand of nucleotides that is substantially similar to 19 to 49 consecutive nucleotides of a sequence selected from the group consisting of SEQ ID NOs 1, 3, 5, 7, 9, 11, 13, 15, 17, 19, 21, and 23 and a second strand that is substantially complementary to the first. In another embodiment, the dsRNA has overhangs of two to ten nucleotides at one or both of the 3′ ends.

In a seventh aspect, the invention provides a purified Aureobasidin A synthetase polypeptide comprising at least 7 (at least 7, 8, 9, 10, 12, 15, 18, or 20) consecutive residues of a sequence selected from the group consisting of SEQ ID NOs 2, 4, 6, 8, 10, 12, 14, 16, 18, and 22. In one embodiment, the polypeptide comprises an immunogenic domain of at least 7 (at least 7, 8, 9, 10, 12, 15, 18, or 20) consecutive residues of a sequence selected from the group consisting of SEQ ID NOs 2, 4, 6, 8, 10, 12, 14, 16, 18, 20 and 22. In another embodiment, the purified polypeptide comprises an amino acid sequence at least 70% (e.g., greater than 70%, 80%, 90%, 95%, 98%, or 99%) identical to a sequence selected from the group consisting of SEQ ID NOs 2, 4, 6, 8, 10, 12, 14, 16, 18, 20 and 22. In yet another embodiment, the purified polypeptide comprises an amino acid sequence with up to 110 (up to 100, 90, 80, 70, 60, 50, 40, 30, 20, or 10) amino acid substitutions, deletions, additions or conservative amino acid substitutions, wherein the amino acid sequence is selected from the group consisting of SEQ ID NOs 8, 12, 14 and 18. In even yet another embodiment, the purified polypeptide comprises an amino acid sequence with up to 1100 (up to 1000, 900, 800, 700, 600, 500, 400, 300, 200, 100, or 50) amino acid substitutions, deletions, additions or conservative amino acid substitutions, wherein the amino acid sequence is SEQ ID NO:2. In a further embodiment, the purified polypeptide comprises an amino acid sequence with up to 90 (up to 80, 70, 50, 60, 40, 30, 20, 10, or 5) amino acid substitutions, deletions, additions or conservative amino acid substitutions, wherein the amino acid sequence is SEQ ID NO: 4. In an additional embodiment, the purified polypeptide comprises an amino acid sequence with up to 48 (up to 40, 35, 30, 25, 20, 15, 10, 5, or 2) amino acid substitutions, deletions, additions or conservative amino acid substitutions, wherein the amino acid sequence is SEQ ID NO: 22. In yet another embodiment, the purified polypeptide comprises an amino acid sequence with up to 150 (up to 140, 120, 100, 80, 60, 40, 20 or 10) amino acid substitutions, deletions, additions or conservative amino acid substitutions, wherein the amino acid sequence is SEQ ID NO: 22.

### BRIEF DESCRIPTION OF THE DRAWINGS

FIG. 1 is an image of a gel using SDS-PAGE illustrating the separation of crude lysates from Tolypocladium niveum and A. pullulans.

FIG. 2 is an image of a Southern blot of A. pullulans BP-1938 genomic DNA.

FIG. 3 is a schematic map of the inserts in cosmid clones 511-19V and 89W.

FIG. 4 illustrates a strategy for sequencing the aba1 gene.

FIG. 5 is a schematic illustration of the domain organization for the sequenced aba1 gene.

FIG. 6 is table illustrating an internal comparison of the biosynthetic modules in aba1.

FIG. 7 is a map of the regulatory region of the aba1 gene.

### BRIEF DESCRIPTION OF SEQUENCES

SEQ ID NO:1 aba1 gene, complete coding sequence
SEQ ID NO:2 ABA protein, complete amino acid sequence
SEQ ID NO:3 aba1.1, CAT(D-Hmp [D-hydroxymethylpentanoic acid] module), nucleic acid sequence
SEQ ID NO:4 aba1.1, CAT (D-Hmp) amino acid sequence.
SEQ ID NO:5 aba1.2, CAMT(val) [L-N-methylvaline] module, nucleic acid sequence
SEQ ID NO:6 aba1.2, CAMT(val) amino acid sequence
SEQ ID NO:7 aba1.3, CAT(phe) [L-phenylalanine] module, nucleic acid sequence
SEQ ID NO:8 aba1.3, CAT(phe) amino acid sequence
SEQ ID NO:9 aba1.4, CAMT(phe) [L-N-methylphenylalanine] module, nucleic acid sequence
SEQ ID NO: 10 aba1.4, CAMT(phe) amino acid sequence
SEQ ID NO:11 aba1.5, CAT(pro) [L-proline] module, nucleic acid sequence
SEQ ID NO:12 aba1.5, CAT(pro) amino acid sequence
SEQ ID NO:13 aba1.6, CAT(aIle) [L-allo-isoleucine] module, nucleic acid sequence
SEQ ID NO:14 aba1.6, CAT(aIle) amino acid sequence
SEQ ID NO:15 aba1.7, CAMT(val) [second L-N-; methylvaline] module, nucleic acid sequence
SEQ ID NO:16 aba1.7, CAMT(val) amino acid sequence
SEQ ID NO:17 aba1.8, CAT(leu) [L-leucine] module, nucleic acid sequence
SEQ ID NO:18 aba1.8, CAT(leu) amino acid sequence
SEQ ID NO:19 aba1.9, CAMT(val) [L-hydroxy-N-methylvaline] module, nucleic acid sequence
SEQ ID NO:20 aba1.9, CAMT(val) amino acid sequence
SEQ ID NO:21 aba1, c-terminal condensation module, nucleic acid sequence
SEQ ID NO:22 aba1, c-terminal condensation module, amino acid sequence
SEQ ID NO:23 5′ regulatory region of the aba1 gene.
SEQ ID NO:24 PCR primer sequence
SEQ ID NO:25 PCR primer sequence
SEQ ID NO:26 PCR primer sequence
SEQ ID NO:27 PCR primer sequence
SEQ ID NO:28 PCR primer sequence
SEQ ID NO:29 PCR primer sequence
SEQ ID NO:30 PCR primer sequence
SEQ ID NO:31 PCR primer sequence
SEQ ID NO:32 PCR primer sequence
SEQ ID NO:33 PCR primer sequence
SEQ ID NO:34 PCR primer sequence
SEQ ID NO:35 PCR primer sequence
SEQ ID NO:36 PCR primer sequence
SEQ ID NO:37 PCR primer sequence
SEQ ID NO:38 PCR primer sequence
SEQ ID NO:39 PCR aba1 gene specific primer
SEQ ID NO:40 PCR aba1 gene specific primer
SEQ ID NO:41 Sequencing primer
SEQ ID NO:42 Sequencing primer
SEQ ID NO:43 Poly-T primer
SEQ ID NO:44 5′-RACE anchor primer
SEQ ID NO:45 5′-RACE anchor primer

### DETAILED DESCRIPTION

### I. Definitions

To facilitate understanding of the invention, a number of terms are defined below

As used herein, an enzyme possessing AbA NRP synthetase-like activity is an enzyme which catalyses the biosynthesis of AbA and structurally related peptides and derivatives.

As used herein the term “stringent conditions” refers to hybridization conditions at 42° C. in 6×SSPE, 50% formamide, 5×Denhardt's solution, and 0.1% SDS, followed by washing three times for 10 minutes in 2×SSC, 0.1% SDS, followed by twice for 30 minutes, in 0.2.times SSC, 0.1% SDS at 65° C.

As used herein the term “reduced stringency conditions” refers to stringent hybridization conditions in which the washing temperature is 60° C.

As used herein, the term “nucleic acid molecule”, “nucleic acid sequence” or “polynucleotide” refers to any nucleic acid containing molecule, including but not limited to, DNA or RNA. The term polynucleotide(s) generally refers to any polyribonucleotide or polydeoxyribonucleotide, which can be unmodified RNA or DNA or modified RNA or DNA. Thus, for instance, polynucleotides, as used herein, refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that might be single-stranded or, more typically, double-stranded or a mixture of single- and double-stranded regions.

In addition, “polynucleotide” as used herein, refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

The term “polynucleotide”, “nucleic acid molecule” or “nucleic acid sequence” includes DNAs or RNAs that contain one or more modified bases. Thus, DNAs or RNAs with backbones modified for stability or for other reasons are “polynucloeotides”, “nucleic acid molecules” or “nucleic acid sequences” as those terms are intended herein.

The terms also encompass sequences that include any of the known base analogs of DNA and RNA. Illustrative examples of such nucleobases include without limitation adenine, cytosine, 5-methylcytosine, isocytosine, pseudoisocytosine, guanine, thymine, uracil, 5-bromouracil, 5-propynyluracil, 5-propynylcytosine, 5-propyny-6-fluoroluracil, 5-methylthiazoleuracil, 6-aminopurine, 2-aminopurine, inosine, diaminopurine, 7-deazaguanine, 7-deazaadenine, 3-deazaguanine, 3-deazaadenine, 8-azaguanine, 8-azaadenine, 7-propyne-7-deazaadenine, 7-propyne-7-deazaguanine, 2-chloro-6-aminopurine, 4-acetylcytosine, 5-hydroxymethylcytosine, 8-hydroxy-N-6-methyladenosine, aziridinylcytosine, 5-(carboxyhydroxyl-methyl) uracil, 5-fluorouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, N6-methyladenine, 7-methylguanine and other alkyl derivatives of adenine and guanine, 2-propyl adenine and other alkyl derivatives of adenine and guanine, 2-aminoadenine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 2-thiothymine, 5-halouracil, 5-halocytosine, 6-azo uracil, cytosine and thymine, 5-methyl-2-thiouracil, 2-thiouracil, 4-thiouracil, 5-methyluracil, 8-halo, 8-amino, 8-thiol, 8-hydroxyl and other 8-substituted adenines and guanines, 5-trifluoromethyl uracil and cytosine, N-uracil-5-oxyacetic acid methylester, uracil-5-oxyacetic acid, queosine, xanthine, hypoxanthine, 2-thiocytosine, 2,6-diaminopurine, 5-substituted pyrimidines, 6-azapyrimidines and N-2, N-6 and O-6 substituted purines, including 2-aminopropyladenine, 5-propynyluracil and 5-propynylcytosine.

Oligonucleotides can also have sugars other than ribose and deoxy ribose, including arabinofuranose (described in International Publication number WO 99/67378, which is herein incorporated by reference), xyloarabinofuranose (described in U.S. Pat. Nos. 6,316,612 and 6,489,465, which are herein incorporated by reference), α-threofuranose (Schöning, et al. (2000) Science, 290, 1347-51, which is herein incorporated by reference) and L-ribofuranose. Sugar mimetics can replace the sugar in the nucleotides. They include cyclohexene (Wang et al. (2000) J. Am. Chem. Soc. 122, 8595-8602; Vebeure et al. Nucl. Acids Res. (2001) 29, 4941-4947, which are herein incorporated by reference), a tricyclo group (Steffens, et al. J. Am. Chem. Soc. (1997) 119, 11548-11549, which is herein incorporated by reference), a cyclobutyl group, a hexitol group (Maurinsh, et al. (1997) J. Org. Chem, 62, 2861-71; J. Am. Chem. Soc. (1998) 120, 5381-94, which are herein incorporated by reference), an altritol group (Allart, et al., Tetrahedron (1999) 6527-46, which is herein incorporated by reference), a pyrrolidine group (Scharer, et al., J. Am. Chem. Soc., 117, 6623-24, which is herein incorporated by reference), carbocyclic groups obtained by replacing the oxygen of the furnaose ring with a methylene group (Froehler and Ricca, J. Am. Chem. Soc. 114, 8230-32, which is herein incorporated by reference) or with an S to obtain 4′-thiofuranose (Hancock, et al., Nucl. Acids Res. 21, 3485-91, which is herein incorporated by reference), and/or morpholino group (Heasman, (2002) Dev. Biol., 243, 209-214, which is herein incorporated by reference) in place of the pentofuranosyl sugar. Morpholino oligonucleotides are commercially available from Gene Tools, LLC (Corvallis Oregon, USA).

The oligonucleotides can also include “locked nucleic acids” or LNAs. The LNAs can be bicyclic, tricyclic or polycyclic. LNAs include a number of different monomers, one of which is depicted in Formula I.

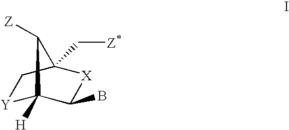

wherein
B constitutes a nucleobase;
Z is selected from an internucleoside linkage and a terminal group;
Z is selected from a bond to the internucleoside linkage of a preceding nucleotide/nucleoside and a terminal group, provided that only one of Z and Z* can be a terminal group;
X and Y are independently selected from —O—, —S—, —N(H)—, —N(R)—, —CH₂— or —C(H)═, CH₂—O—, —CH₂—S—, —CH₂—N(H)—, —CH₂—N(R)—, —CH₂—CH₂— or —CH₂—C(H)═, —CH═CH—;
provided that X and Y are not both O.

In addition to the LNA [2′-Y,4′-C-methylene-β-D-ribofuranosyl] monomers depicted in formula XVIII (a [2,2,1]bicyclo nucleoside), an LNA or LNA* nucleotide can also include “locked nucleic acids” with other furanose or other 5 or 6-membered rings and/or with a different monomer formulation, including 2′-Y,3′ linked and 3′-Y,4′ linked, 1′-Y,3 linked, 1′-Y,4′ linked, 3′-Y,5′ linked, 2′-Y,5′ linked, 1′-Y,2′ linked bicyclonucleosides and others. All the above mentioned LNAs can be obtained with different chiral centers, resulting, for example, in LNA [3′-Y-4′-C-methylene (or ethylene)-β (or α)-arabino-, xylo- or L-ribo-furanosyl] monomers. LNA oligonucleotides and LNA nucleotides are generally described in International Publication No. WO 99/14226 and subsequent applications; International Publication Nos. WO 00/56746, WO 00/56748, WO 00/66604, WO 01/25248, WO 02/28875, WO 02/094250, WO 03/006475; U.S. Pat. Nos. 6,043,060, 6,268,490, 6,770,748, 6,639,051, and U.S. Publication Nos. 2002/0125241, 2003/0105309, 2003/0125241, 2002/0147332, 2004/0244840 and 2005/0203042, all of which are incorporated herein by reference. LNA oligonucleotides and LNA analogue oligonucleotides are commercially available from, for example, Proligo LLC 6200 Lookout Road, Boulder, Colo. 80301 USA.

The nucleotide derivatives can include nucleotides containing one of the following at, the 2′ sugar position: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10, C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group, 2′-dimethylaminooxyethoxy (i.e., an O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, 2′-methoxy (2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide.

In some embodiments, the oligonucleotides have non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Some modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Other modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In yet other oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

In some embodiments, oligonucleotides of the invention are oligonucleotides with phosphorothioate backbones and oligonucleosides with heteroatom backbones, and in particular —CH₂—, —NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Oligonucleotides can also have a morpholino backbone structure of the above-referenced U.S. Pat. No. 5,034,506.

In some embodiments the oligonucleotides have a phosphorothioate backbone having the following general structure.

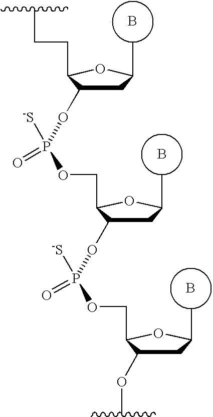

It will be appreciated that a great variety of modifications have been made to DNA and RNA that serve many useful purposes known to those of skill in the art. The term “polynucleotide” as it is employed herein embraces such chemically, enzymatically or metabolically modified forms of polynucleotides, as well as the chemical forms of DNA and RNA characteristic of viruses and cells, including simple and complex cells, inter alia.

The term “isolated” means altered “by the hand of man” from its natural state; i.e., if it occurs in nature, it has been changed or removed from its original environment or both. For example, when used in relation to a nucleic acid, as in “an isolated nucleotide” or “isolated polynucleotide” refers to a nucleic acid sequence that is identified and separated from at least one component or contaminant with which it is ordinarily associated in its natural source. Isolated nucleic acid as such is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As part of or following isolation, a polynucleotide can be joined to other polynucleotides, such as for example DNAs, for mutagenesis studies, to form fusion proteins, and for propagation or expression of the polynucleotide in a host. The isolated polynucleotides, alone or joined to other polynucleotides, such as vectors, can be introduced into host cells, in culture or in whole organisms. Such polynucleotides, when introduced into host cells in culture or in whole organisms, still would be isolated, as the term is used herein, because they would not be in their naturally occurring form or environment. Similarly, the polynucleotides and polypeptides may occur in a composition, such as a media formulation (solutions for introduction of polynucleotides or polypeptides, for example, into cells or compositions or solutions for chemical or enzymatic reactions which are not naturally occurring compositions) and, therein remain isolated polynucleotides or polypeptides within the meaning of that term as it is employed herein.

By “isolated nucleic acid sequence” is meant a polynucleotide that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. The term includes single, double, triple stranded forms of DNA and other forms.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences preceding and following the coding region, (leader and trailer) as well as intervening sequences (introns) between individual coding segments (exons). Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger (mRNA) transcript. The mRNA functions during translation to specify the sequence or order of amino acids in a nascent polypeptide.

“Heterologous” refers to a nucleic acid sequence that either originates from another species or is modified from either its original form or the form primarily expressed in the cell. “Heterologous coding sequence” refers to a nucleic acid sequence that encodes a polypeptide, wherein the nucleic acid sequence originates from another species or is modified from either its original form or the form primarily expressed in the cell.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules, (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region (or upstream region) may contain regulatory sequences such as promoters and enhancers that control or influence the transcription of the gene. The 3′ flanking region may contain sequences that direct the termination of transcription, post-transcriptional cleavage and polyadenylation.

The term “wild type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “oligonucleotide” as used herein is defined as a molecule comprised of two or more deoxyribonucleotides or ribonucleotides, preferably more than three, and usually more than ten. The exact size of an oligonucleotide will depend on many factors, including the ultimate function or use of the oligonucleotide. Oligonucleotides can be prepared by any suitable method, including, for example, cloning and restriction of appropriate sequences and direct chemical synthesis by a method such as the phosphotriester method of Narang et al., 1979, Meth. Enzymol., 68:90-99; the phosphodiester method of Brown et al., 1979, Method Enzymol., 68:109-151, the diethylphosphoramidite method of Beaucage et al., 1981, Tetrahedron Lett., 22:1859-1862; the triester method of Matteucci et al., 1981, J. Am. Chem. Soc., 103:3185-3191, or automated synthesis methods; and the solid support method of U.S. Pat. No. 4,458,066.

As used herein, the terms “an oligonucleotide having a nucleotide sequence encoding a gene” and “polynucleotide having a nucleotide sequence encoding a gene,” means a nucleic acid sequence comprising all or part of the coding region of a gene or in other words the nucleic acid sequence that encodes a gene product. The coding region may be present in ace cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The term “primer” as used herein refers to an oligonucleotide, whether natural or synthetic, which is capable of acting as a point of initiation of synthesis when placed under conditions in which primer extension is initiated or possible. Synthesis of a primer extension product which is complementary to a nucleic acid strand is initiated in the presence of nucleoside triphosphates and a polymerase in an appropriate buffer at a suitable temperature.

The term “primer” may refer to more than one primer, particularly in the case where there is some ambiguity in the information regarding one or both ends of the target region to be synthesized. For instance, if a nucleic acid sequence is inferred from a protein sequence, a “primer” generated to synthesize nucleic acid encoding said protein sequence is actually a collection of primer oligonucleotides containing sequences representing all possible codon variations based on the degeneracy of the genetic code. One or more of the primers in this collection will be homologous with the end of the target sequence. Likewise, if a “conserved” region shows significant levels of polymorphism in a population, mixtures of primers can be prepared that will amplify adjacent sequences. For example, primers can be synthesized based upon the amino acid sequence as set forth in SEQ ID NO:1 and can be designed based upon the degeneracy of the genetic code.

The term “plasmids” generally is designated herein by a lower case p preceded and/or followed by capital letters and/or numbers, in accordance with standard naming conventions that are familiar to those of skill in the art.

Plasmids disclosed herein are either commercially available, publicly available on an unrestricted basis, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention. The properties, construction and use of such plasmids, as well as other vectors, in the present invention will be readily apparent to those of skill from the present disclosure.

The term “restriction endonucleases” and “restriction enzymes” refers to bacterial enzymes that cut double-stranded DNA at or near a specific nucleotide sequence.

As used herein, vector (or plasmid) refers to discrete elements that are used to introduce heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. An expression vector includes vectors capable of expressing DNA that is operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

A coding sequence is “operably linked” to another coding sequence when RNA polymerase will transcribe the two coding sequences into a single mRNA, which is then translated into a single polypeptide having amino acids derived from both coding sequences. The coding sequences need not be contiguous to one another so long as the expressed sequences ultimately process to produce the desired protein.

Nucleic acid sequences which encode a fusion protein of the invention can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, translational stop sites, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the invention (see e.g., Bitter et al., Methods in Enzymology 153:516-544, 1987). For example, when cloning in bacterial systems, inducible promoters such as pL of bacteriophage γ, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention.

In the present invention, the nucleic acid sequences encoding a protein of the invention may be inserted into a recombinant expression vector. The term “recombinant expression vector” refers to a plasmid, virus or other vehicle known in the art that has been manipulated by insertion or incorporation of the nucleic acid sequences encoding the peptides of the invention. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988), baculovirus-derived vectors for expression in insect cells, cauliflower mosaic virus, CaMV; tobacco mosaic virus, TMV. The nucleic acid sequences encoding a fusion polypeptide of the invention can also include a localization sequence to direct the indicator to particular cellular sites by fusion to appropriate organellar targeting signals or localized host proteins. A polynucleotide encoding a localization sequence, or signal sequence, can be used as a repressor and thus can be ligated or fused at the 5′ terminus of a polynucleotide encoding the reporter polypeptide such that the signal peptide is located at the amino terminal end of the resulting fusion polynucleotide/polypeptide. The construction of expression vectors and the expression of genes in transfected cells involves the use of molecular cloning techniques also well known in the art. Sambrook et al., Molecular Cloning—A Laboratory Manual, Cold Spring Harbor Laboratory, Cold Spring Harbor, N.Y., 2001, and Current Protocols in Molecular Biology, M. Ausubel et al., eds., (Current Protocols, a joint venture between Greene Publishing Associates, Inc. and John Wiley & Sons, Inc., most recent Supplement). These methods include in vitro recombinant DNA techniques, synthetic techniques and in vivo recombination/genetic recombination. (See, for example, the techniques described in Sambrook, et al., Molecular Cloning A Laboratory Manual, Cold Spring Harbor Laboratory, N.Y., 2001).

Depending on the vector utilized, any of a number of suitable transcription and translation elements, including constitutive and inducible promoters, transcription enhancer elements, transcription terminators, etc. can be used in the expression vector (see, e.g., Bitter, et al., Methods in Enzymology 153:516-544, 1987). These elements are well known to one of skill in the art.

In yeast and fungi, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; with supplements 2005; Grant, et al., “Expression and Secretion Vectors for Yeast,” in Methods in Enzymology, Eds. Wu & Grossman, 1987, Acad. Press, New York, Vol. 153, pp. 516-544, 1987; Glover, DNA Cloning, Vol. II, IRL Press, Chs. 1-7, 1995; and “Guide to Yeast Genetics and Molecular and Cell Biolog,” Methods in Enzymology, Eds: Guthrie and Fink, Vol. 350, p. 3-623, 2002; Bitter, “Heterologous Gene Expression in Yeast,” Methods in Enzymology, Eds. Berger & Kimmel, Acad. Press, New York, Vol. 152, pp. 673-684, 1987; and Methods in Yeast Genetics, Eds. Amberg et al., Cold Spring Harbor Press, Vols. I and II, 2005. A constitutive yeast promoter such as ADH or LEU2 or an inducible promoter such as GAL may be used (“Cloning in Yeast,” Ch. 3, R. Rothstein In: DNA Cloning Vol. 11, A Practical Approach, Ed. D M Glover, IRL Press, Wash., D.C., 1986). Alternatively, vectors may be used which promote integration of foreign DNA sequences into the yeast chromosome.

An alternative expression system which could be used to express the proteins of the invention is an insect system. In one such system, Autographa californica nuclear polyhedrosis virus (AcNPV) is used as a vector to express foreign genes. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed, see Smith, et al., J. Viol. 46:584, 1983; Smith, U.S. Pat. No. 4,215,051.

By “transformation” or “transfection” is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

By “transformed cell” or “host cell” is meant a cell (e.g., prokaryotic or eukaryotic) into which (or into an ancestor of which) has been introduced, by means of recombinant DNA techniques, a DNA molecule encoding a polypeptide of the invention (i.e., an ABA polypeptide), or fragment thereof.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with DNA sequences encoding a polypeptide of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Preferably, a eukaryotic host is utilized as the host cell as described herein. The eukaryotic cell can be a yeast or fungal cell (e.g., Saccharomyces cerevisiae), or may be a mammalian cell, including a human cell.

A number of methods are used to transform yeast, including treatment with lithium salts, electroporation and transforming spheroplasts. See, e.g., Current Protocols in Molecular Biology, Ed. Ausubel, et al. (Supplements to 2006).

Eukaryotic systems and mammalian expression systems allow for proper post-translational modifications of expressed mammalian proteins to occur. Eukaryotic cells that possess cellular machinery for proper processing of the primary transcript, glycosylation, phosphorylation, and, advantageously secretion of the gene product should be used. Such host cell lines may include but are not limited to yeast and fungal species and strains and eukaryotic cells such as CHO, VERO, BHK, HeLa, COS, MDCK, Jurkat, HEK-293, and WI38.

For long-term, high-yield production of recombinant proteins, stable expression is preferred. Rather than using expression vectors which contain viral origins of replication, host cells can be transformed with the cDNA encoding a fusion protein of the invention controlled by appropriate expression control elements (e.g., promoter, enhancer, sequences, transcription terminators, polyadenylation sites, etc.), and a selectable marker. The selectable marker in the recombinant plasmid confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including but not limited to the herpes simplex virus thymidine kinase (Wigler, et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes can be employed in tk⁻, hgprt⁻ or aprt⁻ cells respectively. Also, antimetabolite resistance can be used as the basis of selection for dhfr, which confers resistance to methotrexate (Wigler, et al., Proc. Natl. Acad. Sci. USA 77:3567, 1980; O'Hare, et al., Proc. Natl. Acad. Sci. USA 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin, et al., J. Mol. Biol. 150:1, 1981); and hygro, which confers resistance to hygromycin (Santerre, et al., Gene 30:147, 1984) genes. Recently, additional selectable genes have been described, namely trpB, which allows cells to utilize: indole in place of tryptophan; hisD, which allows cells to utilize histinol in place of histidine (Hartman & Mulligan, Proc. Natl. Acad. Sci. USA 85:8047, 1988); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987). As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “completely complementary,” for example when used in reference to an oligonucleotide of the present invention refers to an oligonucleotide where all of the nucleotides are complementary to a target sequence (e.g., a gene).

As used herein, the term “partially complementary,” refers to a sequence where at least one nucleotide is not complementary to the target sequence. Preferred partially complementary sequences are those that can still hybridize to the target sequence under physiological conditions. The term “partially complementary” refers to sequences that have regions of one or more non-complementary nucleotides both internal to the sequence or at either end. Sequences with mismatches at the ends may still hybridize to the target sequence.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. Likewise, A substantially complementary sequence or probe will compete for and inhibit the binding (i.e., the hybridization) of a completely complementary nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, “percent homology” of two nucleic acid sequences or of two amino acid sequences is determined using the algorithm of Karlin and Altschul (Proc. Natl. Acad. Sci. USA 87: 2264-2268, 1990) modified as in Karlin and Altschul (Proc. Acad. Natl. Sci. USA 90:5873-5877, 1993). This algorithm is incorporated into the NBLAST and XBLAST programs of Altschul et al. (J. Mol. Biol. 215″ 403-410, 1990). See http://www.ncbi.nlm.nih.gov.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein, the term “Tₘ” is used in reference to the “melting temperature.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5. times SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used in connection with the present invention the term “polypeptide” or “protein” refers to a polymer in which the monomers are amino acid residues which are joined together through amide bonds. When the amino acids are alpha-amino acids, either the L-optical isomer or the D-optical isomer can be used. The term “polypeptide” as used herein is intended to encompass any amino acid sequence and include modified sequences such as glycoproteins. The term “polypeptide” is specifically intended to cover naturally occurring proteins, as well as those which are recombinantly or synthetically synthesized, which occur in at least two different conformations wherein both conformations have the same or substantially the same amino acid sequence but have different three dimensional structures. “Fragments” are a portion of a naturally occurring protein. Fragments can have the same or substantially the same amino acid sequence as the naturally occurring protein. “Substantially the same” or Substantially similar” means that an amino acid sequence is largely, but not entirely, the same, but retains a functional activity of the sequence to which it is related. In general, two amino acid sequences are “substantially the same” or “substantially homologous” if they are at least 85% identical.

As used herein, functional activity refers to an activity or activities of a polypeptide or portion thereof associated with a full-length (complete) protein. Functional activities include, but are not limited to, biological activity, catalytic or enzymatic activity, antigenicity (ability to bind to or compete with a polypeptide for binding to an anti-polypeptide antibody), immunogenicity, ability to form multimers, and the ability to specifically bind to a receptor or ligand for the polypeptide.

Amino acid substitutions, deletions and/or insertions, can be made in ABA or modules thereof provided that the resulting protein exhibits ABA activity or other activity (or, if desired, such changes can be made to eliminate activity). Muteins can be made by making conservative amino acid substitutions and also non-conservative amino acid substitutions. For example, amino acid substitutions that desirably or advantageously alter properties of the proteins can be made. In one embodiment, mutations that prevent degradation of the polypeptide can be made.

Amino acid substitutions contemplated include conservative substitutions, such as those set forth in Table 1, which likely do not eliminate ABA activity. As described herein, substitutions that alter properties of the proteins are also contemplated.

Suitable conservative substitutions of amino acids are known to those of skill in this art and can be made generally without altering the biological activity, for example enzymatic activity, of the resulting molecule. Skilled artisans recognize that, in general, single amino acid substitutions in non-essential regions of a polypeptide do not substantially alter biological activity (see, e.g., Watson et al. Molecular Biology of the Gene, 5th Edition, 2003, The Benjamin/Cummings Pub. Co.). Also included within the definition, is the catalytically active fragment of a SP, particularly a single chain protease portion. Conservative amino acid substitutions are made, for example, in accordance with those set forth in TABLE 1 as follows:

TABLE 1

| Original Residue | Conservative Substitution |
|---|---|
| Ala (A) | Gly, Ser, Abu |
| Arg (R) | Lys, Orn |
| Asn (N) | Gln, His |
| Cys (C) | Ser |
| Gln (Q) | Asn |
| Glu (E) | Asp |
| Gly (G) | Ala, Pro |
| His (H) | Asn, Gln |
| Ile (I) | Leu, Val, Met, Nle, Nva |
| Leu (L) | Ile, Val, Met, Nle, Nva |
| Lys (K) | Arg, Gln, Glu |
| Met (M) | Leu, Tyr, Ile, Nle, Val |
| Ornithine | Lys, Arg |
| Phe (F) | Met, Leu, Tyr |
| Ser (S) | Thr |
| Thr (T) | Ser |
| Trp (W) | Tyr |
| Tyr (Y) | Trp, Phe |
| Val (V) | Ile, Leu, Met, Nle, Nva |

Other substitutions are also permissible and can be determined empirically or in accord with known conservative substitutions.

As used herein, “Abu” is 2-aminobutyric acid; “Orn” is ornithine; Nva is norvaline; Nle is norleucine.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation, for example of possible modifications. The modified peptides can be chemically synthesized, or the isolated gene can be site-directed mutagenized, or a synthetic gene can be synthesized and expressed in bacteria, yeast, baculovirus, tissue culture and so on.

A DNA “coding sequence of” or a “nucleotide sequence encoding” a particular protein is a DNA sequence which is transcribed and translated into an protein when placed under the control of appropriate regulatory sequences.

“Amino acid sequence” and terms such as “polypeptide” or “protein” are not meant to limit the amino acid sequence to the complete, native amino acid sequence associated with the recited protein molecule.

The term “native protein” is used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

A “recombinant” protein or polypeptide refers to proteins or polypeptides produced by recombinant DNA techniques; i.e., produced from cells transformed by an exogenous DNA construct encoding the desired polypeptide (e.g. the Aureobasidin A Synthetase polypeptide of the present invention). “Synthetic” polypeptides are those prepared by chemical synthesis.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, [2001]).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, [2001]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### II. ABA Nucleic Acid, and Polypeptides

In one embodiment, the invention provides an isolated polynucleotide sequence encoding AbA NRP synthetase (ABA) polypeptide. SEQ ID NO:1 includes the complete open reading frame for ABA. An exemplary ABA polypeptide of the invention has an amino acid sequence as set forth in SEQ ID NO:2. Polynucleotide sequences of the invention include DNA, cDNA and RNA sequences which encode AbA NRP Synthetase. It is understood that all polynucleotides encoding all or a portion of AbA NRP Synthetase are also included herein.

The invention also provides for fragments of the aba1 nucleic acid sequence, including the sequences of the modules of aba1. SEQ ID NOs 3, 5, 7, 9, 11, 13, 15, 17, 19 and 21 encode the polypeptides of the modules. These sequences also include DNA, cDNA and RNA sequences which encode ABA modules.

In another embodiment, the invention provides the nucleic acid sequence of the 5′-regulatory region of the aba1 gene. SEQ ID NO:23 includes the 5′-regulatory region.

Such polynucleotides include naturally occurring, synthetic, and intentionally manipulated polynucleotides. For example, the aba1 polynucleotide may be subjected to site-directed mutagenesis. The polynucleotides of the invention also include sequences that are degenerate as a result of the genetic code. There are 20 natural amino acids, most of which are specified by more than one codon. Therefore, all degenerate nucleotide sequences are included in the invention as long as the amino acid sequence of the ABA polypeptide encoded by the nucleotide sequence is functionally unchanged. Also included are nucleotide sequences which encode ABA polypeptides, such as SEQ ID NO: 1. In addition, the invention also includes a polynucleotide encoding a polypeptide having the biological activity of an amino acid sequence of SEQ ID NO:2. However, it is recognized that portions of either SEQ ID NO: 1 or 2 may be excluded to identify fragments of the polynucleotide sequence or polypeptide sequence. For example, fragments of SEQ ID NO:1 at least 20 (at least 25, 24, 23, 22, 21 or 20) nucleotides in length as well as fragments of SEQ ID NO:2 at least 7 (at least 7, 8, 9, 10, 11, 12, 13, 14, 15. 16, 17, 18, 19, or 20) amino acids in length are encompassed by the current invention, so long as they retain some biological activity related to the ABA polypeptide. ABA biological activity includes for example, antigenicity or the ability to synthesize all or part of AbA. The fragments of SEQ ID NO:2 do not include conserved regions of NRPS proteins. In addition, nucleic acids at least 70% identical (at least 70, 75, 80, 85, 90, 95, 96, 97, 98, or 99% identical) to SEQ IDs 1, 3, 5, 7, 9, 11, 13, 15, 17, 19, 21, and 23 are also included in this invention, so long as they encode a polypeptide that retains some biological activity related to the ABA polypeptide.

The polynucleotides of this invention were originally recovered from Aureobasidium pullulans genomic DNA. Thus, the present invention provides a means for isolating similar nucleic acid molecules from other organisms, encoding polypeptides similar to the polypeptides of the present invention. For example, one may probe a gene library with a natural or artificially designed probe using art recognized procedures (see, for example: Current Protocols in Molecular Biology, Ausubel F. M. et al. (EDS.) Green Publishing Company Assoc. and John Wiley Interscience, New York, 1989, 2006). It is appreciated by one skilled in the art that probes can be designed based on the degeneracy of the genetic code to the sequences set forth in SEQ ID NO:2.

The invention includes polypeptides having substantially the same sequence as the amino acid sequence set forth in SEQ ID NO:2 or functional fragments thereof, or amino acid sequences that are substantially the same as SEQ ID NO:2. Thus, the invention includes the amino acid sequences of the modules of ABA set forth in SEQ ID NOs 4, 6, 8, 10, 12, 14, 16, 18, 20, and 22.

A protein having the amino acid sequence of the ABA protein to which one or more amino acid residues have been added is exemplified by a fusion protein containing the protein. Fusion proteins, in which the ABA protein is fused to other peptides or proteins, are included in the present invention. Fusion proteins can be made using techniques well known to those skilled in the art, for example, by linking the DNA encoding the ABA protein (SEQ ID NO:2) in frame with the DNA encoding other peptides or proteins, followed by inserting the DNA into an expression vector and expressing it in a host. Alternatively, the chimeric sequence may be introduced into a host cell by homologous recombination. There is no restriction as to the peptides or proteins to be fused to the protein of the present invention.

For instance, known peptides which may be used for the fusion include the FLAG peptide (Hopp et al., BioTechnology 6:1204-1210, 1988), 6×His that is made up of six histidine residues, 10×His, influenza hemagglutinin (HA), human c-myc fragment, VSV-GP fragment, p18HIV fragment, T7-tag, HSV-tag, E-tag, SV40 T antigen fragment, 1ck tag, alpha-tubulin fragment, B-tag, and Protein C fragment. Also, glutathione-S-transferase t (GST), influenza hemagglutinin (HA), the constant region of immunoglobulin, beta-galactosidase, maltose binding protein (MBP), and the like may be used as a protein to be fused with the protein of this invention. Fusion proteins can be prepared by fusing the DNA encoding these peptides or proteins, which are commercially available, with the DNA encoding the protein of the invention, and expressing the fused DNA.

The proteins of the present invention may have variations in the amino acid sequence, molecular weight, isoelectric point, presence or absence of sugar chains, or form, depending on the cell or host used to produce them or the purification method utilized as described below. Nevertheless, so long as the protein obtained has a function equivalent to the ABA protein, it is within the scope of the present invention. For example, when the inventive protein is expressed in prokaryotic cells, e.g., E. coli, a methionine residue is added at the N-terminus of the original protein. The present invention also includes such proteins.

ABA polypeptides of the present invention include peptides, or full length protein, that contain substitutions, deletions, or insertions into the protein backbone, that would still leave an approximately 70% (75%, 80%, 85%, 90%, 95%, 98% or 99%) homology to the original protein over the corresponding portion. A yet greater degree of departure from homology is allowed if like-amino acids, i.e. conservative amino acid substitutions, do not count as a change in the sequence.

The polynucleotide encoding ABA includes the nucleotide sequences of SEQ ID NO:1 and SEQ ID NO:23 as well as nucleic acid sequences complementary to those sequences. When the sequence is RNA, the deoxyribonucleotides A, G, C, and T of SEQ ID NO:1 are replaced by ribonucleotides A, G, C, and U, respectively. Also included in the invention are fragments (portions) of the above-described nucleic acid sequences that are at least 15 bases in length, which is sufficient to permit the fragment to selectively hybridize to DNA that encodes the protein of SEQ ID NO:2 or similar proteins. “Selective hybridization” as used herein refers to hybridization under moderately stringent or highly stringent conditions (See, for example, the techniques described in Sambrook et al., 2001 Molecular Cloning A Laboratory Manual, Cold Spring Harbor Laboratory, New York, incorporated herein by reference), which distinguishes related from unrelated nucleotide sequences.

Also provided are nucleic acid molecules that hybridize to the above-noted sequences of nucleotides encoding ABA at least at low stringency, at moderate stringency, and/or at high stringency, and that encode the one or part of one of the modules and/or the full length protein. Generally the molecules hybridize under such conditions along their full length (or along at least about 70%, 80% or 90% of the full length) for at least one domain or module and encode at least one domain, such as the condensation domain, of the polypeptide.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

Oligonucleotides encompassed by the present invention are also useful as primers for nucleic acid amplification reactions. In general, the primers used according to the method of the invention embrace oligonucleotides of sufficient length and appropriate sequence which provides specific initiation of polymerization of a significant number of nucleic acid molecules containing the target nucleic acid under the conditions of stringency for the reaction utilizing the primers. In this manner, it is possible to selectively amplify the specific target nucleic acid sequence containing the nucleic acids of interest. Specifically, the term “primer” as used herein refers to a sequence comprising sixteen or more deoxyribonucleotides or ribonucleotides, preferably at least twenty, which sequence is capable of initiating synthesis of a primer extension product that is substantially complementary to a target nucleic acid strand. The oligonucleotide primer typically contains 15-22 or more nucleotides, although it may contain fewer nucleotides as long as the primer is of sufficient specificity to allow essentially only the amplification of the specifically desired target nucleotide sequence (i.e., the primer is substantially complementary).

Amplified products can be detected by Southern blot analysis, with or without using radioactive probes. In such a process, for example, a small sample of DNA containing a very low level of ABA nucleotide sequence is amplified and analyzed via a Southern blotting technique known to those of skill in the art. The use of non-radioactive probes or labels is facilitated by the high level of the amplified signal.

The ABA polynucleotide of the invention is derived from a fungus, Aureobasidium pullulans. Screening procedures that rely on nucleic acid hybridization make it possible to isolate any gene sequence from any organism, provided the appropriate probe is available. For example, it is envisioned that such probes can be used to identify other homologs of the ABA family of enzymes in fungi or, alternatively, in other organisms such as bacteria. To accomplish this, oligonucleotide probes, which correspond to a part of the sequence encoding the protein in question, can be synthesized chemically. This requires that short stretches of amino acid sequence be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of DNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res., 9:879, 1981).

When the entire sequence of amino acid residues of the desired polypeptide is not known, the direct synthesis of DNA sequences is not possible and the methods of choice are the synthesis of cDNA sequences or isolating genomic sequences. Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned.

Amplification, such as PCR, can be carried out by a thermalcycler and thermostable DNA polymerase. The nucleic acid that is amplified can include mRNA or cDNA or genomic DNA from any prokaryotic or eukaryotic species. One can choose to synthesize several different degenerate primers, for use in the PCR reactions. It also is possible to vary the stringency of hybridization conditions used in priming the PCR reactions, to amplify nucleic acid orthologs or homologs by allowing for greater or lesser degrees of nucleotide sequence similarity between the known nucleotide sequence and the nucleic acid homolog being isolated. For cross species hybridization, low or moderate stringency conditions are used. For same species hybridization, moderate or high stringency conditions generally are used. After successful amplification of the nucleic acid containing all or a portion of the identified ABA protein sequence or of a nucleic acid encoding all or a portion of an ABA protein homolog, that segment can be molecularly cloned and sequenced, and used as a probe to isolate a complete cDNA or genomic clone. This, in turn, permits the determination of the gene's complete nucleotide sequence, the analysis of its expression, and the production of its protein product for functional analysis. Once the nucleotide sequence is determined, an open reading frame encoding the ABA protein product can be determined by any method well known in the art for determining open reading frames, for example, using publicly available computer programs for nucleotide sequence analysis. Once an open reading frame is defined, it is routine to determine the amino acid sequence of the protein encoded by the open reading frame. In this way, the nucleotide sequences of the entire SP protein genes as well as the amino acid sequences of ABA proteins and analogs can be identified.

### III. Plasmids Vectors and Cells

Plasmids and vectors comprising the nucleic acid molecules are also provided. Cells containing the vectors, including cells that express the encoded proteins are also provided. The host cell can be prokaryotic or eukaryotic. The cell can be a bacterial cell, a yeast cell, including Saccharomyces cerevisiae or Pichia pastoris, a fungal cell, a plant cell, an insect cell or an animal cell. Methods for producing ABA or portions of the ABA polypeptide are provided herein. For example, growing the cell under conditions whereby the encoded ABA is expressed by the cell, and recovering the expressed protein, are provided.

DNA sequences encoding ABA can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used.

In the present invention, the ABA polynucleotide sequences may be inserted into a recombinant expression vector. The term “recombinant expression vector” refers to a plasmid, virus, artificial chromosome or other vehicle known in the art that has been manipulated by insertion or incorporation of ABA nucleic acid sequences. Such expression vectors contain a promoter sequence which facilitates the efficient transcription of the inserted nucleic acid sequence of the host. The expression vector typically contains an origin of replication, a promoter, as well as specific genes which allow phenotypic selection of the transformed cells. Vectors suitable for use in the present invention include those described above.

Methods which are well known to those skilled in the art can be used to construct expression vectors containing the ABA coding sequence and appropriate transcriptional/translational control signals. These methods include in vitro recombinant DNA techniques, synthetic techniques, and in vivo recombination/genetic techniques. (See, for example, the techniques described in Sambrook et al., 2001, Molecular Cloning A Laboratory Manual, Cold Spring Harbor Laboratory, New York).

The genetic construct can be designed to provide additional benefits, such as, for example, addition of C-terminal or N-terminal amino acid residues that would facilitate purification by trapping on columns or by use of antibodies. All those methodologies are cumulative. For example, a synthetic gene can later be mutagenized. The choice as to the method of producing a particular construct can easily be made by one skilled in the art based on practical considerations: size of the desired peptide, availability and cost of starting materials, etc. All the technologies involved are well established and well known in the art. See, for example, Ausubel et al., Current Protocols in Molecular Biology, Volumes 1-4, with supplements 2006, and Sambrook et al., Molecular Cloning, a Laboratory Manual, Cold Spring Harbor Laboratory (2001). Yet other technical references are known and easily accessible to one skilled in the art.

The ABA polypeptide and its domains, derivatives and analogs can be produced by various methods known in the art. For example, once a recombinant cell expressing an ABA protein, or a domain, fragment or derivative thereof, is identified, the individual gene product can be isolated and analyzed. This is achieved by assays based on the physical and/or functional properties of the protein, including, but not limited to, radioactive labeling of the product followed by analysis by gel electrophoresis, immunoassay, cross-linking to marker-labeled product.

The ABA polypeptides can be isolated and purified by standard methods known in the art, either from natural sources or recombinant host cells expressing the complexes or proteins. The methods include but are not restricted to column chromatography (e.g., ion exchange, affinity, gel exclusion, reversed-phase high pressure and fast protein liquid), differential centrifugation, differential solubility, or by any other standard technique used for the purification of proteins. Functional properties can be evaluated using any suitable assay known in the art.

Manipulations of ABA protein sequences can be made at the protein level. Also contemplated herein are ABA proteins, domains thereof, derivatives or analogs or fragments thereof, which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications can be carried out by known techniques, including but not limited to specific chemical cleavage by cyanogen bromide, trypsin, chymotrypsin, papain, V8 protease, NaBH.sub.4, acetylation, formylation, oxidation, reduction and metabolic synthesis in the presence of tunicamycin.

A variety of modifications of the ABA protein and domains are contemplated herein. An ABA-encoding nucleic acid molecule can be modified by any of numerous strategies known in the art Sambrook et al. (2001) Molecular Cloning: A Laboratory Manual (3rd Ed.), Vol. 1-3, Cold Spring Harbor Laboratory, Cold Spring Harbor, N.Y.). The sequences can be cleaved at appropriate sites with restriction endonuclease(s), followed by further enzymatic modification if desired, isolated, and ligated in vitro. In the production of the gene encoding a domain, derivative or analog of ABA, care should be taken to ensure that the modified gene retains the original translational reading frame, uninterrupted by translational stop signals, in the gene region where the desired activity is encoded.

Additionally, the ABA-encoding nucleic acid molecules can be mutated in vitro or in vivo, to create and/or destroy translation, initiation, and/or termination sequences, or to create variations in coding regions and/or form new restriction endonuclease sites or destroy pre-existing ones, to facilitate further in vitro modification. Also, as described herein, muteins with primary sequence alterations are contemplated. Such mutations can be effected by any technique for mutagenesis known in the art, including, but not limited to, chemical mutagenesis and in vitro site-directed mutagenesis (Hutchinson et al., J. Biol. Chem. 253:6551-6558 (1978)), use of TAB® linkers (Pharmacia). In one embodiment, for example, an ABA protein or domain thereof is modified to include a fluorescent label.

### IV. Antibodies that Bind to ABA

In another embodiment, the present invention provides antibodies that bind to ABA and to specific modules of ABA that may produce cyclic peptides similar to AbA. Such antibodies are useful for research and diagnostic tools to identify organisms that express polypeptides similar to ABA.

The term “epitope”, as used herein, refers to an antigenic determinant on an antigen, such as a ABA polypeptide, to which the paratope of an antibody, such as an ABA-specific antibody, binds. Antigenic determinants usually consist of chemically active surface groupings of molecules, such as amino acids or sugar side chains, and can have specific three-dimensional structural characteristics, as well as specific charge characteristics.

As used herein, the term “immunogenic fragment” or immunogenic domain” means a polypeptide of at least 7 (at least 7, 8, 9, 10, 11, 12, 13, 14, 15, 16, 17, 18, 19, or 20) amino acids in length that can elicit an immune response in an animal.

Antibodies which bind to the ABA polypeptide are well known to those skilled in the art. The antibodies can be prepared using an intact polypeptide or fragments containing small peptides of interest as the immunizing antigen. The polypeptide or a peptide used to immunize an animal can be derived from translated cDNA or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, a chicken or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, updated 2005, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

An antibody suitable for binding to ABA is specific for at least one portion of the ABA polypeptide (SEQ ID NO:2). For example, one of skill in the art can use the peptides to generate appropriate antibodies of the invention. Antibodies of the invention include polyclonal antibodies, monoclonal antibodies, and fragments of polyclonal and monoclonal antibodies.

The preparation of polyclonal antibodies is well-known to those skilled in the art. See, for example, Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, including supplements, 2005, which are hereby incorporated by reference.

The preparation of monoclonal antibodies likewise is conventional and known to those skilled in the art. See, for example, Kohler & Milstein, Nature, 256:495 (1975); Coligan et al., sections 2.5.1-2.6.7; Harlow et al., Antibodies: A Laboratory Manual, page 726 (Cold Spring Harbor Pub. 1988), and Harlow, et al, Using Antibodies: A Laboratory Manual (Cold Spring Harbor Pub. 1999) which are hereby incorporated by reference.

### V. Modulation of aba1 Gene Expression

In another embodiment, the present invention provides a method for modulating aba1 gene expression and well as methods for screening for agents which modulate aba1 gene expression. In one embodiment, the 5′ regulatory region contained in SEQ ID NO:23 may be used to modulate aba1 gene expression. The entire sequence, fragments thereof or the sequence with insertions, substitutions or deletions may be ligated to the coding sequence or fragments thereof to modulate aba1 gene expression. Alternatively, sequences hybridizing to regulatory elements in the 5′-regulatory region of the aba1 gene may be introduced in aba1 gene expressing cells, thereby disturbing the in situ function of the regulatory elements of the aba1 gene.

The 5′ regulatory region may also be used to screen for agents that modulate aba1 gene expression. A number of methods may be employed to screen for and isolate the agents, including gel shift assays and screening cDNA expression libraries for molecules that bind to the 5′ regulatory region or fragments thereof. All the technologies involved are well established and well known in the art. See, for example, Ausubel et al., Current Protocols in Molecular Biology, Volumes 1-4 (2006), with supplements.

The 5′ regulatory region can also be fused to a reporter gene, such as the firefly luciferase gene or the gene encoding chloramphenicol acetyltransferase gene, or other reporter genes. (Alam et al., Anal. Biochem. 188: 245-54 (1990)). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding ABA. By fusing it to the 5′-end of an open reading frame, the 5′-regulatory region can also be used to regulate the expression of essentially any protein expressed in a eukaryotic cell, recombinant as well as endogenous.

Additional assay formats can be used to monitor the ability of the agent to modulate the expression of a nucleic acid encoding ABA. For instance, mRNA expression can be monitored directly by hybridization to the nucleic acids. Cells are exposed to an agent suspected or known to have aba1 gene expression modulating activity. The change in aba1 gene expression is then measured as compared to a control or standard sample. The control or standard sample can be the baseline expression of the cell or subject prior to contact with the agent. An agent which modulates aba1 gene expression may be a polynucleotide for example. The polynucleotide may be an antisense, a triplex agent, a ribozyme, or a double-stranded interfering RNA. For example, an antisense may be directed to the structural gene region or to the promoter region of aba1. The agent may be an agonist, antagonist, peptide, peptidomimetic, antibody, or chemical.

### VI. Screening Assay for Compounds that Affect ABA

In another embodiment, the invention provides a method for identifying a compound which modulates ABA expression or activity including incubating components comprising the compound and a ABA polypeptide, or a recombinant cell expressing a ABA polypeptide, under conditions sufficient to allow the components to interact and determining the affect of the compound on the expression or activity of the gene or polypeptide, respectively. The term “affect”, as used herein, encompasses any means by which aba1 gene expression or protein activity can be modulated. Such compounds can include, for example, polypeptides, peptidomimetics, chemical compounds and biologic agents as described below.

Incubating includes conditions which allow contact between the test compound and ABA, a cell expressing ABA or nucleic acid encoding ABA. Contacting includes in solution and in solid phase. The test ligand(s)/compound may be a combinatorial library for screening a plurality of compounds. Compounds identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/-Technology, 3:1008-1012, 1985), oligonucleotide ligation assays (OLAs) (Landegren, et al., Science, 241:1077, 1988), and the like. Molecular techniques for DNA analysis have been reviewed (Landegren, et al., Science, 242:229-237, 1988).

Thus, the method of the invention includes combinatorial chemistry methods for identifying chemical compounds that bind to ABA or affect ABA expression or activity. By providing for the production of large amounts of ABA, one can identify ligands or substrates that bind to, modulate, affect the expression of, or mimic the action of ABA. For example, a polypeptide may have biological activity associated with the wild-type protein, or may have a loss of function mutation due to a point mutation in the coding sequence, substitution, insertion, deletion and scanning mutations.

A wide variety of assays may be used to screen for compounds that modulate ABA expression or activity, including labeled in vitro protein-protein binding assays, protein-DNA binding assays, electrophoretic mobility shift assays, immunoassays for protein binding, and the like. The purified protein may also be used for determination of three-dimensional crystal structure, which can be used for modeling intermolecular interactions, for example.

The term “agent” as used herein describes any molecule, e.g. protein or pharmaceutical, with the capability of altering or mimicking the physiological function or expression of ABA. Generally, a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, including organic molecules, preferably small organic compounds having a molecular weight of more than 50 and less than about 2,500 daltons. Candidate agents may comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification and amidification to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc. that are used to facilitate optimal protein-protein binding and/or reduce non-specific or background interactions. Reagents that improve the efficiency of the assay, such as protease inhibitors, nuclease inhibitors and anti-microbial agents may be used. The mixtures of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

### VII. Detection of ABA In Vivo and In Vitro

In a further embodiment, the invention provides a method of detecting ABA protein or aba1 nucleic acid in a cell, including contacting a cell component containing aba1 with a reagent which binds to the cell component. The cell component can be nucleic acid, such as DNA or RNA, or it can be protein. When the component is nucleic acid, the reagent is a nucleic acid probe or PCR primer. When the cell component is protein, the reagent is an antibody probe. The probes are detectably labeled, for example, with a radioisotope, a fluorescent compound, a bioluminescent compound, a chemiluminescent compound, a metal chelator or an enzyme. Those of ordinary skill in the art will know of other labels suitable for binding to an antibody or nucleic acid probe, or will be able to ascertain such, using routine experimentation.

For purposes of the invention, an antibody or nucleic acid probe specific for aba1 may be used to detect the presence of ABA polypeptide (using antibody) or polynucleotide (using nucleic acid probe) in biological fluids or tissues. Any cell or cell lysate containing a detectable amount of ABA antigen or polynucleotide can be used.

Another technique which may also result in greater sensitivity consists of coupling antibodies to low molecular weight haptens. These haptens can then be specifically detected by means of a second reaction. For example, it is common to use such haptens as biotin, which reacts with avidin, or dinitrophenyl, pyridoxal, and fluorescein, which can react with specific anti-hapten antibodies.

In another embodiment, nucleic acid probes can be used to identify aba1 or similar nucleic acids from prokaryotic and eukaryotic cells, including fungi and bacteria.

Oligonucleotide probes, which correspond to a part of the sequence encoding ABA or similar molecules can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res. 9:879, 1981).

Hybridization and detection methods are well known to those skilled in the art and are detailed in Sambrook, et al, 2001, and Current Protocols in Molecular Biology as referenced above.

### VIII. Kits for Detection of ABA

The materials for use in the method of the invention are ideally suited for the preparation of a kit. Such a kit may comprise a carrier means being compartmentalized to receive one or more container means such as vials, tubes, and the like, each of the container means comprising one of the separate elements to be used in the method. For example, one of the container means may comprise an ABA binding reagent, such as an antibody or nucleic acid. A second container may further comprise ABA polypeptide. The constituents may be present in liquid or lyophilized form, as desired.

One of the container means may comprise a probe which is or can be detectably labeled. The probe may be an antibody or nucleotide specific for a target protein, or fragments thereof, or a target nucleic acid, or fragment thereof, respectively, wherein the target is indicative, or correlates with, the presence of ABA. For example, oligonucleotide probes of the present invention can be included in a kit and used for examining the presence of aba1 nucleic acid.

The kit may also contain a container comprising a reporter-means, such as a biotin-binding protein, such as avidin or streptavidin, bound to a reporter molecule, such as an enzymatic, fluorescent, or radionucleotide label to identify the detectably labeled oligonucleotide probe

Where the kit utilizes nucleic acid hybridization to detect the target nucleic acid, the kit may also have containers containing nucleotide(s) for amplification of the target nucleic acid sequence. When it is desirable to amplify the target nucleic acid sequence, such as an aba1 nucleic acid sequence, this can be accomplished using oligonucleotide(s) that are primers for amplification. These oligonucleotide primers are based upon identification of the flanking regions contiguous with the target nucleotide sequence.

The kit may also include a container containing antibodies which bind to a target protein, or fragments thereof. Thus, it is envisioned that antibodies which bind to ABA, or fragments thereof, can be included in a kit.

### IX. aba1, the Gene Encoding the AbA Non-Ribosomal Peptide Synthetase Complex

Maximally accurate identification and characterisation of the module and domain sequences of the AbA synthetase, at both the enzymatic and genetic levels, constitutes the basis for a well-directed genetic engineering effort aimed at altering the NRPS complex' specificity for the in vivo production of (novel) AbA variants.

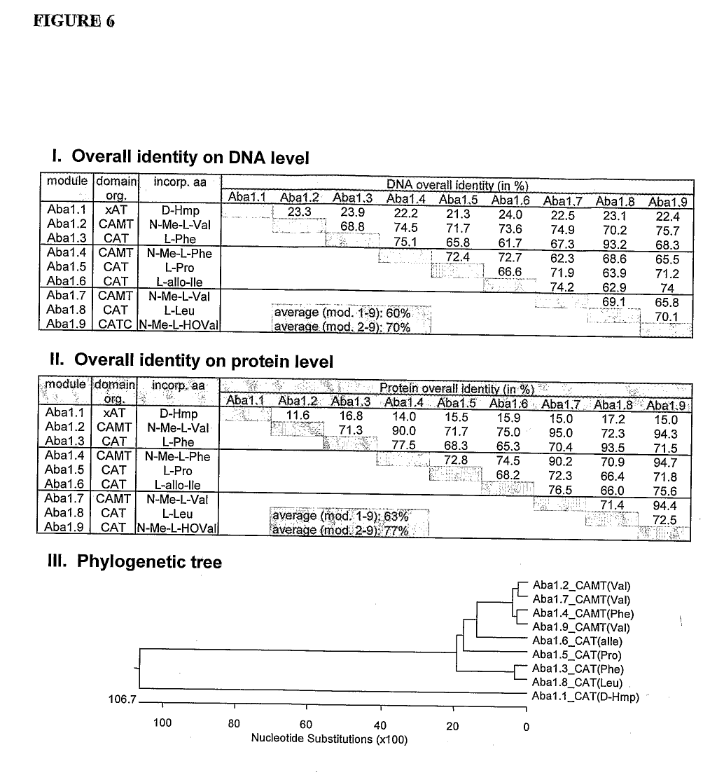

The Aureobasidin A NRPS gene (aba1) encodes nine separate modules, spanning 34,980 bp, and the identities of the respective modules are as predicted from the structure of AbA. The aba1 gene is similar in organization to NRPS genes isolated from other fungi: its transcript is a single mRNA that encodes a single large polypeptide (1.3 million Daltons). Unexpectedly, the aba1 gene has a high degree of shared identity among its component biosynthetic modules, both at the nucleotide and amino acid levels (FIG. 6).

Most of the modules share more than 70% amino acid identity with another module in the complex, and modules with the same amino acid specificity share up to 95% identity. (See FIG. 6 and below). In addition, extensive regions (1800 bp) within the sequence from module 2 to 9, share nearly 100% nucleotide sequence identity. When sequencing the aba1 gene, this very high degree of shared identity required the generation of 15 subclones, from the original cosmid clones, to obtain the complete sequence. The high degree of shared identity (among the modules) is significantly different from what has been found in other fungal NRPS genes. For example, the modules in the HC-toxin NRPS gene, htsI, share at best 37% amino acid sequence identity and although the level of identity is higher in the cyclosporin biosynthesis complex gene, cssA, it does not exceed 60%.

By internal sequence comparisons of the derived amino acid sequences and the correlation of specific partial sequences, modules or domains of the AbA synthetase catalyzing activation of the individual amino acids, condensation, thiolation, methylation and epimerization, may be localized.

In other embodiments, the aba1 gene is used to transform organisms that are not capable of producing AbA into AbA producing organisms and, again by transformation, as a means of increasing gene copy numbers, and thereby AbA production levels, in AbA producing organisms.

In yet other embodiments, the 5′ regulatory domain of the aba1 can be altered, for example by using approaches known in the art to introduce heterologous promoter elements, for the purpose of increasing the ABA producing capacity of an AbA producer organism. Alternatively, the aba1 gene 5′ regulatory domain itself may be used as a promoter element to drive the expression of a heterologous gene in A. pullulans, or a heterologous organism.

A further use of the isolated aba1 gene is for gene-specific mutagenesis. Instead of producing mutations in the entire genome—and therefore also altering many uninvolved genes—the isolated gene alone is mutated, using suitable methods, and then transformed to Aureobasidium pullulans. Among the transformants, the proportion of mutants in the aba1 gene is higher than with mutagenesis of the fungus. Mutants, which specifically form AbA in greater or reduced quantities, may more frequently be found than with conventional mutagenesis.

In further embodiments, fragments of the aba1 gene, most notably individual domain and module encompassing fragments, are used for engineering of both the aba1 gene itself and heterologous NRPS complex genes. An important purpose for this is the generation of organisms capable of producing novel cyclic peptides with novel, most notably pharmaceutical, properties. Such aba1 gene fragments can be expressed as individual enzymes and used for example for the in vitro assembly of (cyclic) peptides.

The aba1 gene and/or fragments thereof are useful as probes for the identification of novel aba-related NRPS genes. When screening for microorganisms capable of synthesizing AbA, an important consideration is that the active metabolites screened for are formed in sufficient quantity. Moreover substances with slightly changed characteristics may be overlooked. The isolated AbA synthetase gene can be used to find microorganisms which contain the AbA synthetase gene, as well as related genes, in their genome. These genes may or may not be active. On the basis of such hybridisation experiments, genes related to aba1 may be isolated in a manner known in the art and transformed into Aureobasidium pullulans. A strain may be used to this end which does not contain any active AbA synthetase gene. This interspecific recombination cannot be achieved with other methods. In this case, genetic variability is based on the introduced gene which hybridizes with the AbA synthetase (aba1) gene.

The isolated aba1 gene can act as an analytical aid in order to determine whether a specific strain of Aureobasidium pullulans has a high concentration of aba1 mRNA. Such strains may then be subjected to conventional mutagenesis and strain selection. Even if the initial strain used for transformation is not limited in its AbA synthetase activity, a strain is provided in this way which potentially allows increased AbA formation. The combination of classical genetics (mutation and strain selection) with molecular genetics (transformation with isolated genes) allows the isolation of improved strains which could not be achieved by either of the two methods alone: not by classical genetics because a double mutation is extremely rare in a single selection stage; not by molecular genetics because in some circumstances an unknown factor has a limiting effect.

The regulatory sequences in the AbA synthetase gene may also be used in expression constructs. Strains of Aureobasidium pullulans which are transformed with plasmids containing these sequences permit, not only the selection of regulatory mutants, but moreover make it possible to measure and optimise promoter activity independently of other functions.

All references cited herein are incorporated in their entirety.

Without further elaboration, it is believed that one skilled in the art can, using the preceding description, utilize the present invention to its fullest extent. The following examples are to be considered illustrative and thus are not limiting of the remainder of the disclosure in any way whatsoever.

### Example 1

### Isolation of Active AbA Synthetase

Although the AbA producing A. pullulans strain R-106 has been studied for some time, little is known about the gene encoding the synthetase responsible for the production of AbA, i.e. the AbA NRPS complex gene (aba1). NRPS complexes in other cyclic peptide-producing fungi examined to date, however, have been found to consist of a single, in some cases quite large protein, encoded by a single gene. Based on these observations and the number of amino acids in Aureobasidin A, the predicted size of the reading frame for the AbA NRPS complex gene should be approximately 37 kb, corresponding to a NRPS complex with a molecular mass of approximately 1.3 million Daltons. Biochemical studies indicate that the ABA protein may indeed be similar to NRPS complexes in other fungi. SDS-PAGE separations of crude and fractionated lysates from an AbA producing Aureobasidium strain shows that this strain contains a very high molecular mass protein that migrates at a position similar to the cyclosporine synthetase complex (sim A) in Tolypocladium nivaeum.

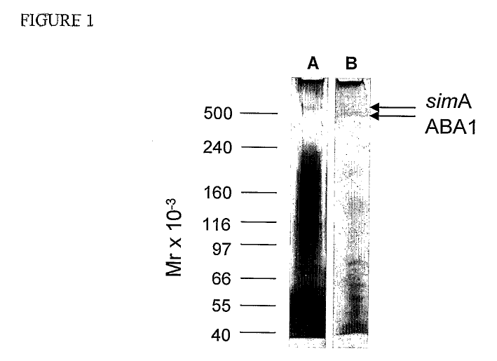

FIG. 1 illustrates the separation of crude A. pullulans (A) and Tolypocladium niveum (B) lysate on SDS-PAGE. The arrows indicate the positions of the putative AbA NRPS complex and sim A. Although the observed, apparent molecular mass of both proteins, at 500-700 kDa, is far below the predicted mass of the AbA NRPS complex (and sim A), it is consistent with the anomalously low apparent, 600-700 kDa molecular mass observed previously for the 1.6 million Dalton cyclosporine synthetase, upon separation on SDS-PAGE.

### Example 2

### Identification of an A. pullulans Strain Containing a Single Copy of the aba1 Gene

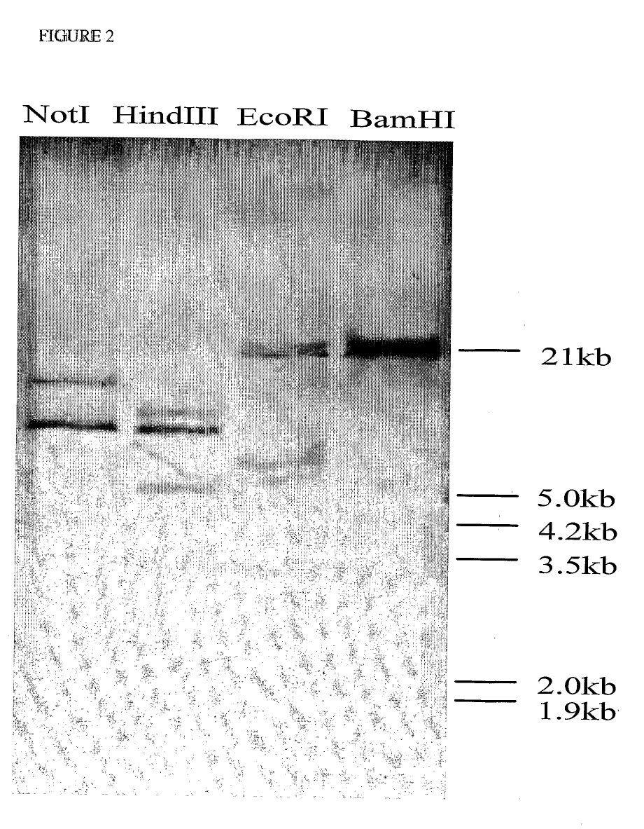

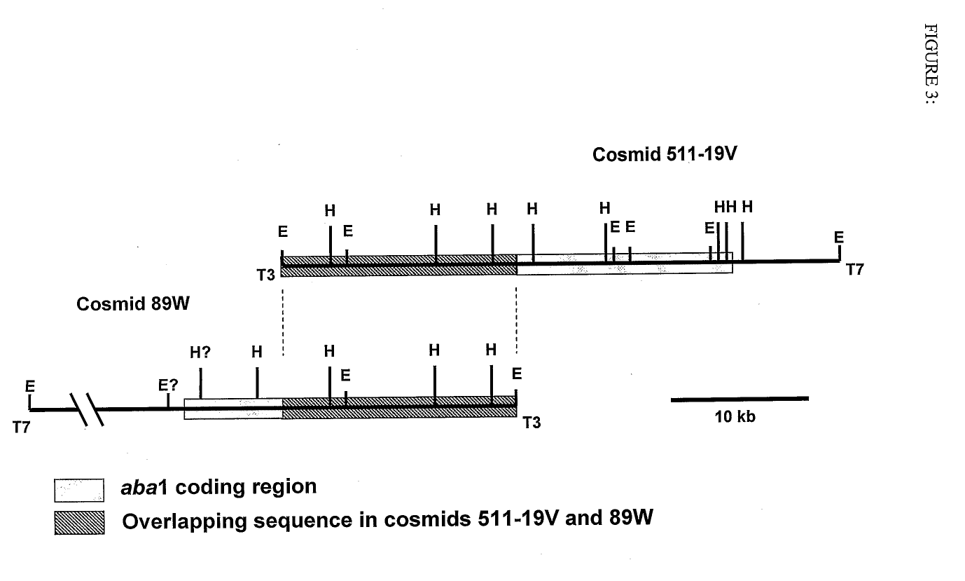

The AbA producer strain does not contain a large number of aba1 gene copies. Comparatively few single nucleotide polymorphisms (SNPs) were identified when sequencing (fragments of) the gene and the number of NRPS positive clones isolated from the lambda and cosmid genomic libraries were low. A tentative assumption was made that the producer strain likely contains no more than two gene copies. The assumption of a low aba1 gene copy number in the AbA producer strain was confirmed by the Southern blotting analysis shown in FIG. 2. The restriction enzyme banding patterns revealed by this blot are consistent with that found within the cosmid clones and later the complete sequence of the aba1 gene. The results from these experiments also indicated that it is unlikely that the genome of the AbA producer strain contains any other closely related NRPS genes. The clones obtained did not contain any other NRPS-related sequences and re-hybridization of the blot under low stringency conditions did not result in any changes in the hybridization pattern, which would be expected if other cross-hybridizing gene sequences were present.

### Example 3

### Design of Degenerate Primers and PCR Amplification of Regions of the A. pullulans aba1 Gene

Several bacterial and fungal NRPS complex genes have been cloned and sequenced and comparative analyses find that regions of these genes share a significant degree of similarity. Although the exact amino acid sequences encoding specific domains vary within genes and among genes from different species, all functional NRPS complexes contain domain core sequences that are well conserved. Consensus sequences have been derived for these conserved cores and these sequences have been used for design of degenerate primers that have been used to isolate novel NRPS complex genes. A general cloning approach, using degenerate primers based on conserved core sequences in NRPS adenylation and thiolation domains, has been described by Turgay, K., and Marahiel, M. A. (1994). This approach involves amplification of a DNA segment that spans part of the adenylation domain and the adjacent thiolation domain. Because one of the primers used is specific for a conserved thiolation domain motif, this approach has the ability to distinguish true NRPS sequences from other adenylate forming enzyme genes.

Advances in the design of degenerate primers for the amplification of sequences from distantly related genes include a procedure referred to as the COnsensus-DEgenerate Hybrid Oligonucleotides (CODEHOP) strategy. See Rose, T. M., Schultz, E. R., Henikoff, J. G., Pietrokovski, S., McCallum, C. M., Henikoff, S. (1998). Consensus-degenerate hybrid oligonucleotide primers for amplification of distantly related sequences. (Nucl. Acids Res. 26:1628-1635.) This strategy involves the alignment of amino acid sequences from the deduced distantly related proteins using Clustal W to obtain alignment blocks. The blocks are then searched for conserved cores from which a computer program designs modified degenerate primers. These modified degenerate primers differ from “normal” degenerate primers in that they contain two parts, a 5′-consensus clamp which is common to all designed primers and a 3′-degenerate core that is designed from the amino acid alignment. The expectation is that these primers will have a much higher probability to amplify related gene sequences from more distantly related species. Computer programs needed for the CODEHOP strategy are available at (http://blocks.fhcrc.org/codehop.html). The CODEHOP approach appears to be ideally suited for amplification of novel NRPS genes because these genes sequences contain conserved motifs from which the CODEHOP program can readily design modified degenerate primers.

CODEHOP was used to identify conserved blocks and design primers for the amino acid sequences extending 500 amino acid residues upstream (towards the N-terminus) from the thiolation “T” conserved core (through the adenylation “A2” core domain) of several bacterial and fungal NRPS genes (for conserved core nomenclature, see Marahiel, M. A., Stachelhaus, T., and Motz, H. D., 1997). The quality of these alignments was found to be much better when bacterial or fungal NRPS proteins were grouped separately. Because the goal was to design degenerate primers to amplify NRPS gene regions from the fungus A. pullulans, it was decided to use only the fungal NRPS gene sequences in the CODEHOP alignment. The fungal-derived NRPS protein sequences used for this alignment are listed in Table 2.

TABLE 2
Source of fungal NRPS genes, gene names or numbers, and module number (Th#).

| | | | GenBank |
|---|---|---|---|
| Species | Gene Name | Thiolation #s | Number |
| Alternaria alternate | AM-toxin synthetase | Th1, Th2 | AAF01762 |
| Emericella nidulans | ACV synthetase | Th1, Th2, Th3 | CAA38631 |
| Aspergillius nidulans | AN0016.2 | Th1 | EAA65335 |
| Cochliobolus | HC-toxin | Th4 | M98024 |
| carbonum | | | |
| Gibberella zeae PH-1 | FG05372.1 | Th1 | XP_385548 |
| Kallichroma tethys | Alpha-aminoadipyl- | Th1, Th2, Th3 | AAK21902 |
| | cysteinyl-valine synthetase | | |
| Leptosphaeria | SirP synthetase | Th1 | AAS92545 |
| maculans | | | |
| Metarhizium anisopliae | peptide synthetase | Th3, Th4 | CAA61605 |
| Tolypocladium | cyclosporine synthetase | Th5, Th8 | CAA82227 |
| inflantum | | | |
| Ustilago maydis | Ferrichrome siderophore | Th1 | O43103 |
| | peptide synthetase | | |

GenBank accession numbers are for the complete NRPS gene sequence.

The individual protein sequences were submitted to CODEHOP which first generated motif blocks from the most conserved core regions within the alignment, and interestingly, many of the identified motif blocks encompassed the highly conserved core motifs described by Marahiel et al. (1997). CODEHOP was then used to design degenerate primers for the consensus cores, “A3, A7, and T”. The design and orientation of these modified degenerate primes are listed in Table 3.

TABLE 3

NRPS motifs used for CODEHOP primer design and primer
sequences. Nucleotide sequences include the IUPAC-IUB
codes for nucleotide ambiguities.

| | | | |
|---|---|---|---|
| Core | Primer number | Primer sequence | Orientation |

| | | | |
|---|---|---|---|
| A3 | AUG003-A3FWD | 5′-CCGGCACCACCggnaarcchaa-3′ | Forward |
| | | [SEQ ID NO: 24] | |
| A3 | AUG004- | 5′-TCACCTCCGGCACCachggnaarcc-3′ | Forward/ |
| | A3FWD2 | [SEQ ID NO: 25] | backup |
| A7 | AUG005-A7FWD | 5′- | Forward |
| | | GTCCACGGACGGATGTACarrachggvga- | |
| | | 3′[SEQ ID NO: 26] | |
| A7 | AUG006-A7RVS | 5′-CCGGACCATGTCGccngtbykrta-3′ | Reverse |
| | | [SEQ ID NO: 27] | |
| T | AUG007-ThioRvs | 5′-GCTGCATGGCGGTGATGswrtsnccbcc- | Reverse |
| | | 3′[SEQ ID NO: 28] | |

In most cases the degenerate primer pairs used generated DNA fragment bands that included a band of the expected size (Table 4). The DNA amplicon mixture obtained, using the “A3 to T” degenerate primers, was used as template for re-amplification, using the primer combinations listed in Table 4. Most of these primer pairs generated DNA fragment sets that included a fragment of the expected size. The fact that the degenerate primer sets derived from the internal core motifs do amplify DNA fragments from the “A3 to T” amplicon mixture strongly suggests that the original “A3 to T” amplification product(s) contain sequences that indeed were derived from a NRPS module(s).

The amplicons from the A7/T re-amplification experiment were cloned into the Invitrogen™ TOPO TA cloning vector, and numerous clones were isolated and sequenced. The sequences obtained were subjected to database searches which showed that one of the clones contained a NRPS-related sequence that was different from the 10 kb NRPS-related sequence previously isolated from A. pullulans by Peery et al. (1997). This clone, designated aug005-aug007, contained a 500 bp NRPS-related sequence. To verify the authenticity of the sequence in aug005-aug007, specific primers (aug016-aug017) were designed to amplify a sequence segment immediately internally to the 5′ and 3′ ends of the 500 bp sequence.

TABLE 4
Expected and observed DNA bands obtained using degenerate
primer pairs and genomic template DNA from A. pullulans.

| Core | | Expected Bands | |
|---|---|---|---|
| Motif | | (w/o and w M | |
| Span | Primer Set | domain, in kb) | Observed Bands |
| A3/T | AUG003 [SEQ ID | 1.2/2.4 | 0.35, 0.50, 0.80, 1.20, |
| | NO: 24]-AUG007 | | 1.50, 1.80 |
| | [SEQ ID NO: 28] | | |
| A7/T | AUG005 [SEQ ID | 0.6/1.8 | None |
| | NO: 26]-AUG007 | | |
| | [SEQ ID NO: 28] | | |
| A3/A7 | AUG003 [SEQ ID | 0.7/0.7 | 0.35, 0.40, 0.60, 0.90, |
| | NO: 24]-AUG006 | | 1.10, 1.50, 2.50 |
| | [SEQ ID NO: 27] | | |

Expected bands include modules with and without M domains. M domains add about 1 kb between the A3, A7/T domains.

TABLE 5
Expected and observed DNA bands obtained using degenerate
primer pairs and template DNA from the A3-thiolation
amplicon mixture (re-amplification).

| Core | | Expected Bands | |
|---|---|---|---|
| Motif | | (w/o vs. w/M | |
| Span | Primer Set | domain, in kb) | Observed Bands |
| A7/T | AUG005 [SEQ ID | 0.6/1.8 | 0.30, 0.40, 0.50, 0.65, |
| | NO: 26]-AUG007 | | 0.80, 0.85 |
| | [SEQ ID NO: 28] | | |
| A3/A7 | AUG003 [SEQ ID | 0.7/0.7 | 0.20, 0.30, 0.50, 0.65, |
| | NO: 24]-AUG006 | | 1.40 |
| | [SEQ ID NO: 27] | | |

Using these primers, amplification from genomic A. pullulans DNA yielded the expected 488 bp DNA fragment, suggesting that the 500 bp fragment was derived from a bona fide NRPS gene and hence that it would be an appropriate probe when screening cDNA and genomic clone banks for the aba1 (and other NRPS) gene(s). [In fact later comparison of this 488 bp sequence with the completed aba1 gene sequence [SEQ ID NO:1] revealed that it closely matches sequences in the L-Pro module (positions 14866 to 18102): it shares 93.5% identity from positions 16678 to 17166 in the L-Pro module contained within SEQ ID NO: 1. Most of the sequence differences (27/32) are located in the first 20 bp and last 20 bp of the match and thus may result from the use of degenerate primers.]

A subsequent experiment utilized primers extending in the 5′ and 3′ directions, outward from the 488 bp NRPS-related sequence. Assuming that several of the modules within the aba1 gene share a considerable amount of sequence identity, it was expected that such forward and reverse primers would yield DNA amplicons that spanned from one module to the next, i.e. in the range of 3-4 kb. The experiment did yield DNA fragments that were in the range of 3.3 kb and these fragments were cloned into the TOPO TA cloning vector. Five clones were selected for sequence analyses. This revealed that four of the five clones (designated C5, C8, C9 and C10) contained 3.2 to 3.3 kb inserts that shared identity with NRPS-related genes. The sequences of clone C5 and C9 are identical. It was later found that these clones share identity with the following regions of SEQ ID NO: 1: C5 and C9, 16678 to 20011; C8, 20027 to 23263; and C10, 27746 to 30988.

The NRPS motif searches of the sequences (in these clones) strongly suggested that they all contained the motif organization expected for a complete NRPS module. Attempts were made to expand on the four clones by (PCR) amplifying the regions between each of the putative module clones. This resulted in inserts as large as 6 kb. Sequence analysis of these clones was difficult because the inserts appeared to be comprised of duplicated sequence segments. The PCR-amplification approach did yield several NRPS-related cloned sequences and these sequences were useful when screening cDNA and genomic libraries for larger fragments of the aba1 gene.

### Example 4

### Cloning of aba1 Gene from A. pullulans BP-1938 Using Reverse Transcriptase-PCR (RT-PCR)

The isolation of cDNA clones has the advantage that any NRPS-related sequences obtained would be derived only from expressed NRPS genes. As discussed above, it is known that the AbA producer strain generates considerable amounts of ABA and also that it expresses a protein with an apparent molecular mass that is similar to that predicted for the AbA NRPS complex (see above). Thus, any clones derived from A. pullulans cDNA would be much more likely (than those amplified from genomic DNA) to be derived from the aba1 gene. In addition, the use of N-terminal (derived from the protein sequence) and poly(A) specific primers would allow for the isolation of the corresponding regions of the aba1 gene. Total RNA was isolated using a TRI reagent kit from Molecular Research Center, Inc. and converted to cDNA, using a random primer kit from Invitrogen. The cDNA was used as template for PCR amplifications, using primers that span the sequence derived from subclones C5/C9, C8, and C10, primers derived from the N-terminal protein sequence and from the poly(A) primer sequence (5′-TTTTTTTTTTTTTTTTTTTTTTTTTV-3′) [SEQ ID NO:43]. Amplifications with these primers yielded DNA fragments of different sizes, although none of the fragments appeared to be larger that 6 kb. Several of the unique DNA fragments were cloned using the TOPO TA cloning vector. This resulted in the following clones, which were all sequenced completely: 75T1-11b (2.7 kb), 74T2-7b (3.2 kb), 75-1-30b (5.3 kb), 74/2-45 (1.6 kb), 74-1-32b (2.3 kb), 74-2-42 (2.3 kb), and 75-1-53 (5.3 kb). The sequencing data revealed that all of the (cDNA) clones except one, 75-T1-11b, contained a NRPS-related sequence. The cloned sequences shared identity to the following regions of SEQ ID NO:1: 74T2-7b, 10522 to 13697; 75-1-30b, 5186 to 10510; 72/2-46, 11193 to 12799; 74-1-32b, 29203 to 31477; 74-2-42, 29203 to 31460; and 75-1-53, 23865 to 29192. The clones that contained NRPS-related sequences shared considerable identity with each other, in particular clones 75-1-30b and 75-1-53. Remarkably, even though these clones are nearly identical subsequent work revealed that they are derived from separate parts of the aba1 gene. Analysis for NRPS domain motifs in the larger cDNA clones suggested that each of them contained approximately one and a half NRPS module. Although each cloned sequence shares a considerable amount of sequence identity, no sequence was 100% identical to any of the other. These findings are consistent with what was found using (degenerate primer) PCR-cloning, in that many of the cloned sequences appeared to be very similar, but not identical. It again suggested that the modules that make up the aba1 gene share a considerable amount of identity (with each other), and that if this indeed was the case, using a PCR cloning strategy for isolation of the entire aba1 gene would be very difficult. As a consequence, to obtain the larger gene fragments needed for cloning of the entire gene, lambda and cosmid libraries were prepared from A. pullulans genomic DNA.

### Example 5

### Cloning of aba1 Gene from A. pullulans BP-1938 from Lambda, and Cosmid Libraries

A. Construction of an A. pullulans Genomic Lambda DNA Library

Two lambda libraries were constructed, the first by cloning genomic Sau3A fragments into the Stratagene™ vector LambdaDashII/BamHI, and the second by using the Stratagene™ vector Lambda FixII/XhoI partial fill-in. The libraries yielded 40,000 and 10,000 clones, respectively, and both were screened using PCR primers aug016 (5′-AGCCTTCTGCCACAAGCCTTGCCTA-3-[SEQ ID NO:29]) and aug017 (5′-AGCATCGCGTGAGTCGAGACGATCT-3′ [SEQ ID NO:30]), which amplifies the 488 bp fragment described above. Numerous clones were isolated and partially sequenced from both libraries. One clone, #S15-aug16-T3, was found to contain a NRPS-related sequence, and sequencing of this clone revealed that it contained about 2.7 kb of the aba1 gene. This cloned sequence share identity with the region between positions 10747 to 13499 in SEQ ID NO: 1.

B. Construction of a Genomic A. pullulans Cosmid DNA Library

A. pullulans DNA, in the size range of about 70 kb, was isolated to facilitate the construction of a cosmid library. The DNA was subjected to a limited Sau3A digestion and cloned into the Stratagene™ cosmid vector, SuperCos1. A total of about 1000 cosmid clones were obtained and subjected to PCR screening using the primers aug524 (5′-ACCGCTTTGTGCAGGTCTCC-3′ [SEQ ID NO:31]) and aug529, (5′-CAAGTGTGTAAGTAGTACTGATG-3′ [SEQ ID NO:32]) both of which are derived from the insert sequence in the clone, 75-1-53, described above. Aug524 and aug529 were selected because, based on the available sequence data, they appeared to be unique for the amplification of a 4 kb amplicon, an assumption that was validated using genomic A. pullulans DNA. The cosmid library was screened in pools of several hundred clones. This yielded a single clone, designated 511-19V. Preliminary sequence analysis of this clone, using the flanking cosmid primers (T3 and T7) and primers aug526 (5′-AATCTATGAAGTCAAAGCGG-3′ [SEQ ID NO:33]), 527 (5′-CCGCTTTGACTTCATAGATTG-3-[SEQ ID NO:34]), 528 (5′-TCAGTACTACTTACACACTTG-3′ [SEQ Id NO:35]), 529 and 466 (5′-AACGTGCTCTTCGCGACCGAG-3′ [SEQ ID NO:36]) resulted in NRPS-related sequences from all primers except for that generated by the T7 primer. The T3 sequence was found to match the 3′-end of the lambda clone S15-aug16-T3, indicating that cosmid 511-19V did not contain the N-terminal region of the aba1 gene. Hence, a second screen of the cosmid library was carried out using primers aug68 (5′-TCGCGTATCAGCTCCCGATTCAGCG-3′ [SEQ ID NO:37]) and aug72 (5′-CGTCTTGTCTCTGCCAGAGAGC-3′ [SEQ ID NO:38]), both of which are derived from sequences upstream of aug524 and aug529. Aug68 and aug72 span the sequence segment between c5 and S15-aug16-T3, and, consistent with this, generate an amplicon of 650 bp. The second cosmid library screen resulted in the isolation of a second cosmid clone, designated 89W. Initial sequencing of this clone, using cosmid flanking primers and some internal primers, indicated that the insert in this clone overlapped, to a significant extent, with the insert in cosmid 511-19V. The full extend of this overlap was determined once sequencing of the inserts of both cosmids 511-19V and 89W was completed.

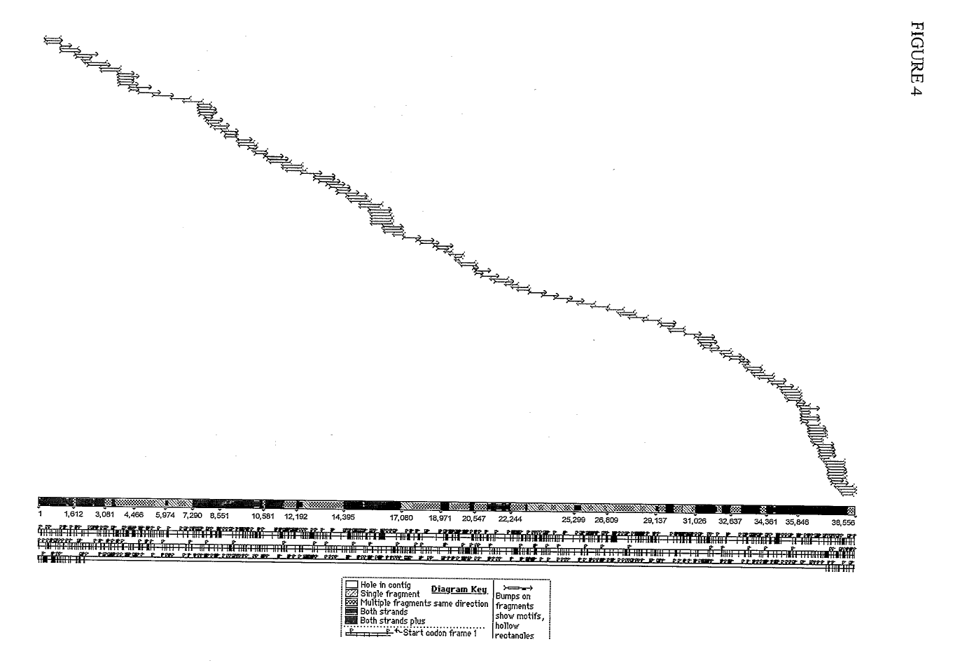

The first attempts to sequence cosmid 511-19V utilized the primers that were used to sequence the PCR generated aba1 clones. However, it soon became evident, that this cosmid clone could not be sequenced directly by conventional primer walking, or a shotgun strategy. This was due to the fact that, consistent with the findings in the PCR and RT-PCR cloning experiments discussed above, many of the modules in the insert shared extensive regions (in the range of 2 kb) of nucleotide sequence identity. Thus, to allow sequencing, subclones needed to be generated from the insert in cosmid 511-19V. EcoRI and HindIII fragments from cosmid 511-19V were prepared, subcloned, mapped, and partially sequenced. The order of these fragments, and their position in the insert, was determined using linking primers (i.e. primers designed to hybridize with sequences flanking the cloning site and to prime across the cloning site) to obtain sequence directly from the intact cosmid and thereby the identity of neighboring subclones. About one half of these linking primers generated readable sequence data, and the other half generated data that appeared to be derived from multiple priming sites. The sequence data, together with data from gel mapping experiments were used to generate EcoRI and HindIII maps of the entire cosmid 511-19V, as well as part of cosmid 89W. See FIG. 3, in which T3 and T7 indicate the location of the cosmid priming sites. H and E indicate the location of the HindIII and EcoRI sites, respectively, in the 511-19V and 89W insert sequences.

### Example 6

### Sequencing and Mapping of the aba1 Gene

A. Sequencing of the aba1 Gene

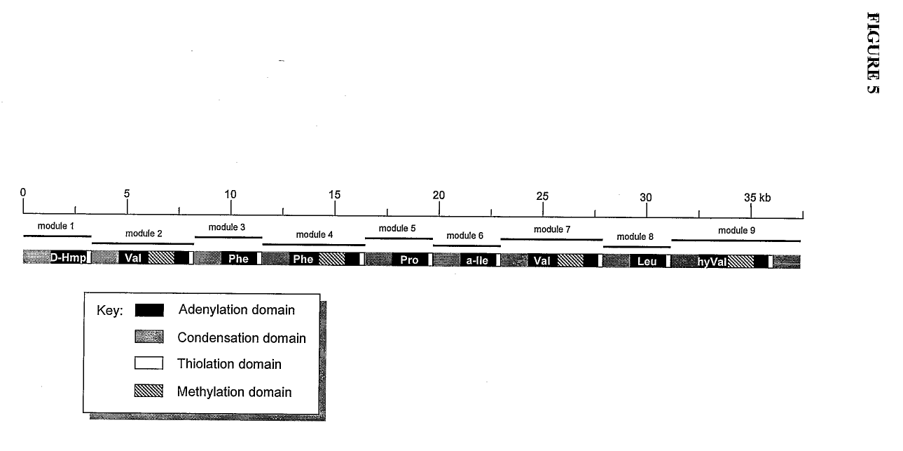

Each of the subcloned EcoRI and HindIII fragments indicated in FIG. 3 were sequenced completely, on both DNA strands. The sequences were subsequently assembled into the complete sequence of the aba1 gene, using overlapping EcoRI and HindIII subclones, or linker sequence derived from the cosmid using primers that extend outward from the 5′ and 3′ flanking ends of the sequence data derived from the subclones, as described above. The cosmid 511-19V was sequenced in its entirety and this revealed that it contains an insert composed of 38,460 bp. The complete sequence of the insert in cosmid 89W was later determined to be 37,495 bp. Cosmid 89W contains about 23 kb of the aba1 gene sequence which includes the aba1 gene promoter, as well as all of the module 1 and module 2 sequence that are missing in cosmid 511-19V. Cosmid 89W also shares a 15,668 bp overlap with cosmid 511-19V (See FIG. 3). The sequencing strategy used to obtain the complete sequence of the A. pullulans aba1 gene is shown in FIG. 4. The sequence data revealed that aba1 gene consists of a single (no introns) open reading frame (ORF) of 34,980 bp that encodes an 11,659 amino acid protein, with a calculated molecular mass of 1,286,254 Daltons. A near consensus Kozak (1999) start site exists at the putative 5′-end of the ORF. This site has the sequence AAGATGC, which is close to the ideal Kozak consensus sequence of A/GXXATGG/A.

Data from other fungal genes suggest that the 5′-flanking region of the (aba1) gene may contain sequence elements that closely match a consensus TATAA element. Examination of the 5′-regulatory portion of the aba1 gene sequence (SEQ. ID. NO 23) revealed that TATAA-related sequences do exist upstream from the consensus ATG, at positions −86 (TATCA), −241 (TATAC), −290 (TATAGC) and −511 (TATAA). Likewise, potential CCAAT elements exist at positions −127 (CAAT), −305 (CAATA), −341 (CAAAT), and −589 (CAACT). This suggest that the aba1 gene contains two (putative) promoter regions and thereby two (putative) transcription start sites, at −71 and −248. 5′-RACE PCR experiments generated fragments ending at both (putative) sites suggesting that both sites may in fact be used by the producer organism (SEQ. ID. NO 23).

B. Mapping of the Biosynthetic NRPS Modules Encoded within the aba1 Gene

The amino acid sequence deduced from the aba1 gene was analyzed for consensus NRPS motifs, such that each domain could be mapped within each of the individual biosynthetic modules in the molecule. Consistent with the composition of AbA, a total of 9 specific modules were mapped within the sequence. Each module is separated from neighboring modules by linker sequences that, in contrast to the module sequences themselves, appear to be unique, with the exception of the linker sequences for modules 4 and 8 which are identical. The module map for the aba1 gene is shown in FIG. 5 and the module positions within the ORF are listed in Table 5. The modules are arranged in the following order: position 1, D-Hmp (SEQ ID NO. 3); 2, N-Me-L-Val (SEQ ID NO. 5); 3, L-Phe (SEQ ID NO. 7); 4, N-Me-L-Phe (SEQ ID NO. 9); 5, L-Pro (SEQ ID NO. 11); 6, L-allo-Ile (SEQ ID NO. 13); 7, N-Me-L-Val (SEQ ID NO. 15); 8, L-Leu (SEQ ID NO. 17); and 9, N-Me-L-HOVal (SEQ ID NO. 19).

TABLE 6
Module identity and location within the aba1 gene. The “no match” entries in the
Predicted substrate column indicate that no exact NRPS module match was found using
current data bases (NCBI, Expasy). The assignments in parenthesis indicate the closest
match.

| | | | | | N- | Predicted |
|---|---|---|---|---|---|---|
| | | Position within | | | methylation: | substrate |
| | Incorporated | aba1 gene | | Domain | expected/ | (Stachelhaus, |
| Module | amino acid | 5′-end | 3′-end | organization | found | 1999) |
| 1 | D-Hmp | 1 | 2682 | (C)AT | −/− | No match (Hiv) |
| 2 | N-Me-L-Val | 2683 | 7143 | CAMT | +/+ | Val |
| 3 | L-Phe | 7144 | 10398 | CAT | −/− | Tyr, Phe |
| 4 | N-Me-L-Phe | 10399 | 14865 | CAMT | +/+ | Tyr, Phe |
| 5 | L-Pro | 14866 | 18102 | CAT | −/− | No match (Ser) |
| 6 | L-allo-Ile | 18103 | 21354 | CAT | −/− | No match (Val) |
| 7 | N-Me-L-Val | 21355 | 25821 | CAMT | +/+ | Val |
| 8 | L-Leu | 25822 | 29079 | CAT | −/− | Leu |
| 9 | N-Me-L- | 29080 | 34977 | CAMT | +/+ | Val |
| | HOVal | | | (C) | | |

The aba1 gene is similar in organization to NRPS genes isolated from other fungi: its transcript is a single mRNA that encodes a single large polypeptide (1.3 million Daltons). Unexpectedly, the aba1 gene has a high degree of shared identity among the biosynthetic modules, both at the nucleotide and amino acid levels. Most of the modules share more than 70% amino acid identity with another module in the complex and modules with the same amino acid specificity share up to 95% identity. (See FIG. 6, in which Panels I and II show sequence identities shared between the modules in aba1, as determined using the nucleotide and amino acid sequences, respectively. Panel III depicts the internal relatedness of the biosynthetic modules in aba1.) In addition, extensive regions (1600 bp) within the sequence from module 2 to 9 share nearly 100% nucleotide identity. This high degree of shared identity (among the modules) is significantly different from what has been found in other fungal NRPS genes. For example, the modules in HC-toxin NRPS gene, htsI share at best 37% amino acid sequence identity and although in the cyclosporin biosynthesis complex gene, cssA, the level of identity is higher, it does not exceed 60% (Scott-Craig et al., 1992; Weber et al., 1994).

### Example 7

### Mapping the aba1 Transcriptional Start Site(s) Using 5′-Rapid Amplification of cDNA Ends (5′-RACE)

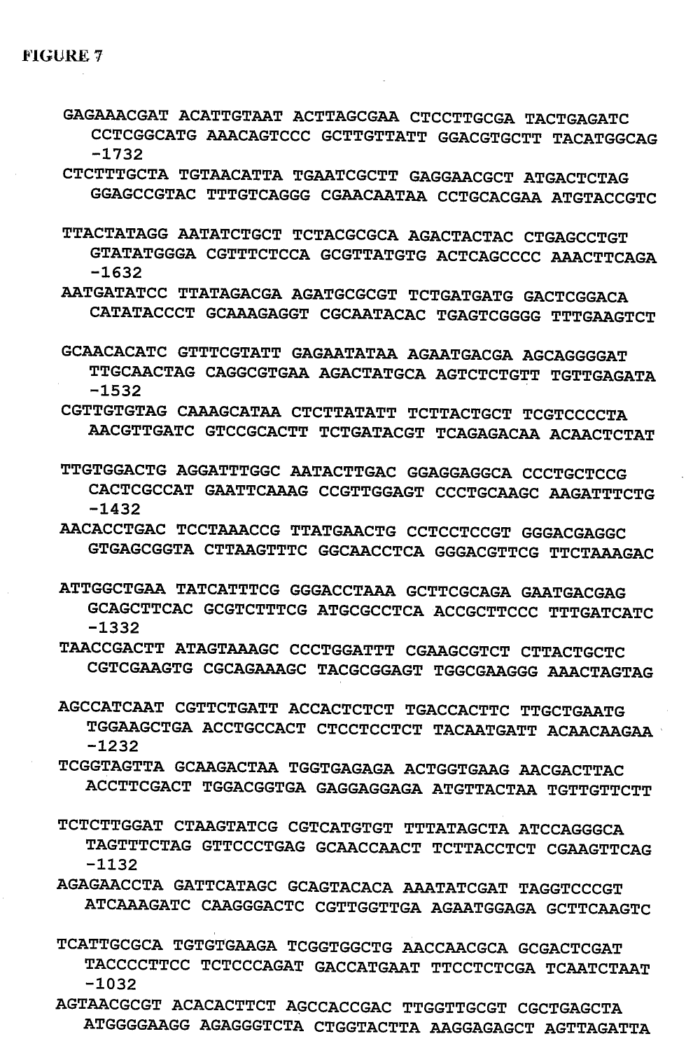

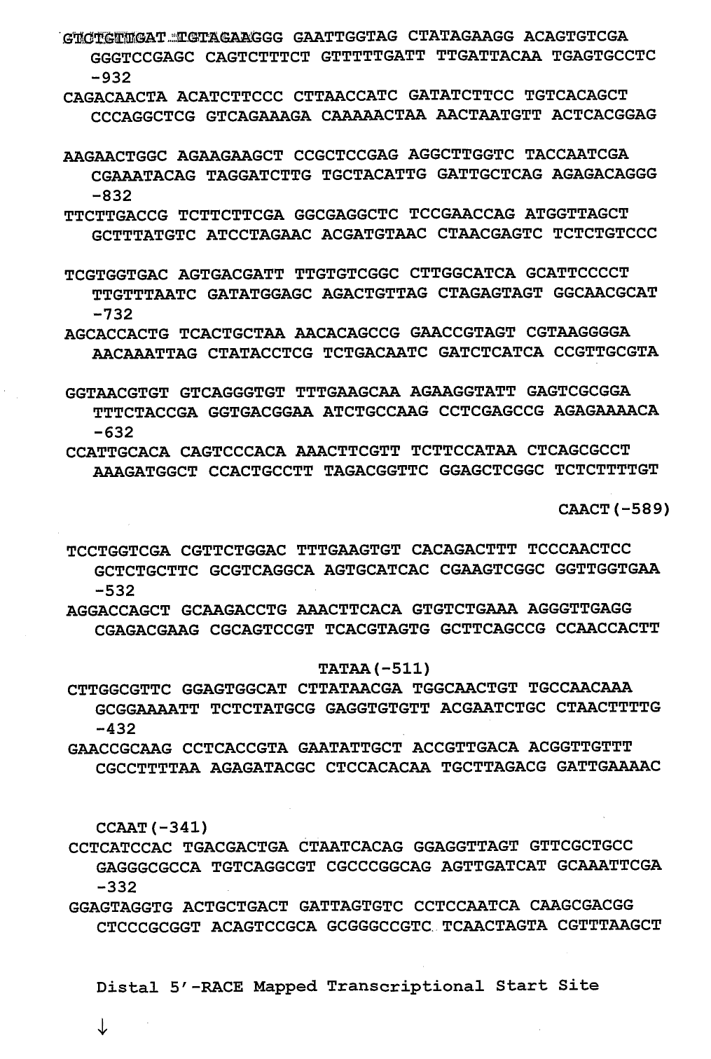

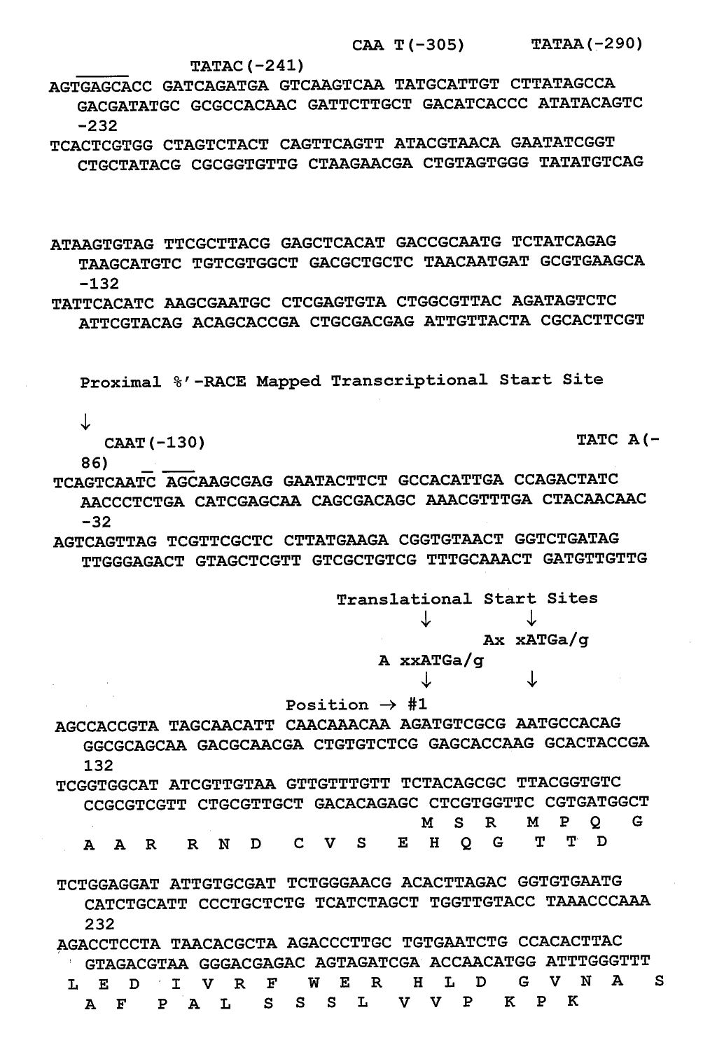

Total A. pullulans RNA was isolated, using a TRI reagent kit from Molecular Research Center, Inc. The 5′-end of the aba1 mRNA was converted to cDNA using a gene specific primer (GSP) AUG901 (5′-TGGATCGAAAGCGCGAGCTG-3′ [SEQ ID NO:39]), which binds to the aba1 gene 700 bp downstream from the first Met codon (position #1 in Seq ID NO: 1), and the 5′-RACE System for Rapid Amplification of cDNA Ends, Version 2.0 (Invitrogen™, Cat. No. 18374-058). After copying the mRNA into cDNA using SuperScript™ reverse transcriptase, the RNA part of the duplex was degraded with RNase. The cDNA was then purified using the spin cartridge supplied in the kit, and 5′-RACE anchor primer (5′-(CUA)4GGCCACGCGTCGACTAGTACGGGIIGGGIIG-3′ [SEQ ID NO:44]) was added (to the purified cDNA) using recombinant Terminal deoxynucleotidyl Transferase (TdT). PCR amplification of the resulting anchor primer extended cDNA was accomplished using 5′-RACE Abridged Anchor Primer (5′-GGCCACGCGTCGACTAGTACGGGIIGGGIIGGGIIG-3′ [SEQ ID NO:45]) and a nested GSP2 primer (AUG1141, 5′-TGTTCTCCAAGTCGAGAATG-3′ [SEQ ID NO:40]). The amplicons derived from this PCR amplification were separated on a 1% agarose gel, which revealed the presence of three distinct DNA fragments, 500, 550, and 800 bp in length). All three DNA fragments were purified using Invitrogen™ gel extraction columns (cat. no. K1999-25) and then directly sequenced, using the primers, AUG1141, AUG142 (5′-ATCCAGGCCGATCGCGCTG-3-[SEQ ID NO:41]), and AUG929 (5′-AGAATCGCACAATATCCTCCAG-3′ [SEQ ID NO:42]). The sequences derived from the 5′-RACE cDNA fragments revealed the presence of two distinct transcription start sites, the first being located at position −72 and the second at position −249, upstream from the translational start site, which is the first codon in SEQ. ID NO:1. The locations of the transcriptional start sites are also shown in SEQ. ID. NO:23 along with the regulatory sequences that are present in the aba1 gene promoter. (See FIG. 7. The location of transcriptional and translational start sites and regulatory elements, such as TATA and CAAT boxes are indicated above the corresponding sequence segments.)

### Example 8

### Increasing the Expression Level of the aba1 Gene through the Use of Heterologous Promoters and Increases in Gene Copy Number

From studies of the nonribosomal biosynthesis of β-lactam antibiotics (e.g. penicillin), which like AbA, are produced by filamentous fungi, it is known that the expression of the ACV NRPS synthetase gene, acvA, is rate limiting for the organism's overall productivity. Kennedy and Turner (1996) clearly demonstrated this by showing that when replacing the weak acvA gene promoter with the strong inducible ethanol dehydrogenase promoter they could increase the acvA gene expression levels up to 100 times. The overexpression of acvA gene alone accounted for a 30-fold increase in penicillin production.

A similar replacement of the endogenous aba1 gene promoter with a strong inducible promoter should be quite feasible. Constitutive (S. cerevisiae) promoters, such as the PAM promoter (plasma membrane H⁺-ATPase; Mahanty et al., 1994), the gpd promoter (glyceraldehydes-3-phosphate dehydrogenase; Nitta et al., 2004) and inducible promoters such as the GALI promoter (galactokinase; Yocum et al., 1984) and AOX1 promoter (alcohol oxidase; Invitrogen™ product K1740-01) have been used successfully previously to increase the expression of heterologous genes in both yeast and filamentous fungi. The substitution of the aba1 gene promoter with any of these heterologous promoters can be accomplished using a gene replacement strategy. One strategy is to place the aba1 5′-flanking DNA sequences from cosmid 89W upstream of the heterologous promoter and place the aba1 gene sequence downstream of the heterologous promoter. When doing this, the translational start site (ATG) can be changed from AAGATGTCG to AAGATGAGC which would still encode Ser as the second amino acid in the polypeptide. The resulting new translational start site matches the consensus site described by Kozak (1999), A/GXXATGGIA, and should result in increased expression.

Abbreviations:

AbA Aureobasidin A

ABA the Aureobasidin A synthesizing NRPS complex (synthetase protein)

aba1 the Aureobasidin A synthesizing NRPS complex gene

ACV aminoadipyl-cysteinyl-valine

amdS acetamidase gene

ATCC American Type Culture Collection

ATP adenosine triphosphate

bp base pairs

CBS Centraalbureau voor Schimmelcultures

DTE dithioerythritol

DTT dithiothreitol

EDTA ethylenediaminetetraacetic acid

HEPES N-2-hydroxyethyl-puperazine-N-2-propanesulphonic acid

MOPS 3-morpholinepropanesulphonic acid

PEQ polyethylene glycol

pfu plaque forming units

SDS sodium dodecyl sulphate

SDS-PAGE SDS-polyacrylamide gel electrophoresis

SSC 150 mM NaCl, 15 mM sodium citrate, pH 7.0

SSPE 180 mM NaCl, 10 mM sodium phosphate, 1 mM EDTA, pH 7.7

TE 10 mM tris-Cl pH 7.5, 1 mM EDTA

TFA trifluoroacetic acid

tris tris(hydroxymethyl)aminomethane

YAC yeast artificial chromosome

Moreover, the customary abbreviations for the restriction endonucleases are used (Sau3A, HindIII, EcoRI, HindIII, ClaI etc.; Sambrook et al., 2001). The nucleotide abbreviations A, T, C, G are used for DNA sequences and the amino acid abbreviations (Arg, Asn, Asp, Cys etc.; or R, N, D, C etc.) for polypeptides (Sambrook et al., 2001).

### REFERENCES

Erdeniz, N., Mortensen, U. H. and Rothstein, R. (1997). Cloning-free PCR-based allele replacement methods. Genome Res. 7:1174-1183.
Gutierrez, S., Diez, B., Montenegro, E., and Martin, J. F. (1991). Characterization of the Cephalosporium acremonium pcbAB gene encoding alpha-aminoadipyl-cysteinyl-valine synthetase, a large multidomain peptide synthetase: linkage to the pcbC gene as a cluster of early cephalosporin biosynthetic genes and evidence of multiple functional domains. J. Bacteriol. 173:2354-2365.
Kennedy, J., and Turner, G. (1996). δ-(L-α-aminoadipyl)-L-cysteinyl-D-valine synthetase is the rate-limiting enzyme for penicillin production in Aspergillus nidulans. Mol. Gen. Genet. 253:189-197.
Kozak, M. (1999). Initiation of translation in prokaryotes and eukaryotes. Gene 234:187-208.
Kurome, T., and Takesako, K. (2000). SAR and potential of the aureobasidin class of antifungal agents. Curr. Opin. Anti-Infect. Invest. Drugs 2:375-386.
Lawen, A., and Zocher, R. (1990). Cyclosporin synthetase. The most complex peptide synthesizing multienzyme polypeptide so far described. J. Biol. Chem. 265:11355-11360.
MacCabe, A. P, Riach, M B, and Kinghorn, J. R. (1991). Identification and expression of the ACV synthetase gene. J. Biotechnol. 17:91-97.
Maniatis, T., Fritsch, E. F., and Sambrook, J. Molecular Cloning. A Laboratory Manual. Cold Spring Harbor Laboratory, Cold Spring Harbor, N.Y., 1982.
Marahiel, M. A., Stachelhaus, T., and Mootz, H. D. (1997). Modular peptide synthetases involved in nonribosomal peptide synthesis. Chem. Rev. 97:2651-2693.
Mootz, H. D., Marahiel, M. A. (1997). The tyrocidine biosynthesis operon of Bacillus brevis: Complete nucleotide sequence and biochemical characterization of functional internal adenylation domains. J. Bacteriol. 179:6843-6850.
Peery, R. B., Thorenwall, S. J. Tobin, M. B., and Skatrud, P. L. (1997). Aureobasidin pullulans cosmid pPSR-22 hydroxylase, multidrug resistance-like protein (ApMDR1), and peptide synthetase genes. GenBank Accession # U85909.
Rose, T. M., Schultz, E. R., Henikoff, J. G., Pietrokovski, S., McCallum, C. M., and Henikoff, S. (1998). Consensus-degenerate hybrid oligonucleotide primers for amplification of distantly related sequences. Nucl. Acids Res. 26:1628-1635.
Rothstein, R. J. (1983). One-step gene disruption in yeast. In Methods in Enzymology (ed. R. Wu, L. Grossman, and K. Moldave), pp. 202-211. Academic Press, New York, N.Y.
Rouhiainen, L., Paulin, L., Suomalainen, S., Hyytiäinen, H., Buikema, W., Haselkorn, R., and Sivonen, K. (2000). Genes encoding synthetases of cyclic depsipeptides anabaenopeptilides in Anabena strain 90. Mol. Microbiol. 37:156-167.
Sambrook, J., McCallum, P., and Russell, D. (2001). Molecular Cloning: A Laboratory Manual, Cold Spring Harbor Press, NY.
Schneider, A., Stachelhaus, T., and Marahiel, M. A. (1998). Targeted alteration of the substrate specificity of peptide synthetases by rational module swapping. Mol. Gen. Genet. 257:308-318.
Scott-Craig, S. J, Panaccione, D. G, Pocard, J.-A. and Walton, J. D. (1992). The cyclic peptide synthetase catalyzing HC-toxin production in the filamentous fungus Coclhliobolus carbonum is encoded by a 15.7-kilobase open reading frame. J. Biol. Chem. 267:26044-26049.
Smith, D. J, Earl, A. J, and Turner, G. (1990). The multifunctional peptide synthetase Performing the first step of penicillin biosynthesis in Penicillium chrysogenum is a 421,073 dalton protein similar to Bacillus brevis peptide antibiotic synthetases. EMBO J. 9:2743-2750.
Stachelhaus, T., Schneider, A., and Marahiel, M. A. (1995). Rational design of peptide antibiotics by targeted replacement of bacterial and fungal domains. Science 269:69-72.
Stachelhaus, T., Mootz, H. D., and Marahiel, M. A. (1999). The specificity-conferring code of adenylation domains in nonribosomal peptide synthetases. Chem. Biol. 6:493-505.
Takesako, K., Kuroda, H., Inoue, T., Haruna, F., Yoshikawa, Y., and Kato, I., (1993). Biological properties of Aureobasidin A, a cyclic depsipeptide antiflmgal antibiotic. J. Antibiot. 46:1414-1420.
Turgay, K., Krause, M., and Marahiet, M. A. (1992). Four homologous domains in the primary structure of GrsB are related to domains in a superfamily of adenylate-forming enzymes. Mol. Microbiol. 6:529-546.
Turgay, K., and Marahiel, M. A. (1994). A general approach for identifying and cloning peptide synthetase genes. Peptide Res. 7:238-240.
Wang, J., Holden, D. W. and Leong, S. A. (1988). Gene transfer system for the phytopathogenic fungus Ustilago maydis. Proc. Natl. Acad. Sci. USA 85:865-869.
Weber, G., Schoergendorfer, K., Schneider-Scherzer, E., and Leitner, E. (1994). The peptide synthetase catalyzing cyclosporine production in Tolypocladium niveum is encoded by a giant 45.8-kilobase open reading frame. Curr. Genet. 26:120-125.
Weckermann, R., Furbass, R., and Marahiel, M. A. (1988). Complete nucleotide sequence of the tycA gene coding the tyrocidine synthetase 1 from Bacillus brevis. Nucleic Acids Res. 16:11841.
Yakimov, M. M., Giuliano L., Timmis, K. N., and Golyshin, P. N. (2000). Recombinant acylheptapeptide lichenysin: high level of production by Bacillus subtilis cells. J. Mol. Microbiol. Biotechnol. 2:217-224.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims.

1. An isolated nucleic acid comprising a sequence that hybridizes under stringent conditions to a hybridization probe, the nucleotide sequence of which hybridization probe consists of a sequence selected from SEQ ID NO: 1, SEQ ID NO:3, SEQ ID NO:5, SEQ ID NO:7. SEQ ID NO:9, SEQ ID NO:1, SEQ ID NO:13, SEQ ID NO:15, SEQ ID NO:17, SEQ ID NO:19, SEQ ID NO:21, SEQ ID NO:23, the complement of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, or SEQ ID NO:23.

2. An isolated nucleic acid according to claim 1 wherein the hybridization probe comprises a fragment of SEQ ID NO: 1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, the complement of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, or SEQ ID NO:23 of at least 50 nucleotides in length.

3. An isolated nucleic acid according to claim 1 wherein the hybridization probe comprises a nucleotide sequence that encodes an enzyme that has Aureobasidin A synthetase activity, encodes an enzyme that catalyzes the biosynthesis of Aureobasidin A and related molecules, or that encodes a biosynthetic module of the enzyme that catalyzes the biosynthesis of Aureobasidin A and related molecules or has Aureobasidin A synthetase activity, the module selected from the group consisting of D-hydroxymethylpentanoic acid module, L-N-methylvaline module, L-phenylalanine module. L-N-methylphenylalanine module, L-proline module, L-allo-isoleucine module, second L-N-methylvaline module, L-leucine module, L-hydroxy-N-methylvaline module, and C-terminal condensation domain of Aureobasidin A synthetase or a combination of modules selected from D-hydroxymethylpentanoic acid module, L-N-methylvaline module, L-phenylalanine module, L-N-methylphenylalanine module, L-proline module, L-allo-isoleucine module, second L-N-methylvaline module, L-leucine module, L-hydroxy-N-methylvaline module, and C-terminal condensation domain of Aureobasidin A synthetase.

4. An isolated nucleic acid comprising a sequence at least 70% identical to SEQ ID NO: 1, wherein the nucleic acid encodes a polypeptide that has Aureobasidin A synthetase activity, or catalyzes the synthesis of Aureobasidin A and related molecules.

5. (canceled)

6. An isolated nucleic acid comprising a sequence that encodes a polypeptide comprising the sequence SEQ ID NO:2 or SEQ ID NO:2 with up to 1100 conservatives amino acid substitutions, wherein the polypeptide has Aureobasidin A synthetase activity or catalyzes the synthesis of Auroebasidin A and related molecules.

7. An isolated nucleic acid comprising a sequence that encodes a polypeptide comprising an immunogenic fragment of SEQ ID NO:2 at least 8 residues in length.

8. An isolated DNA, the nucleotide sequence of which consists of SEQ ID NO:1, SEQ ID NO:3, SEQ ID NO:5, SEQ ID NO:7, SEQ ID NO:9, SEQ ID NO: 11, SEQ ID NO: 13, SEQ ID NO: 15, SEQ ID NO: 17, SEQ ID NO: 19, and SEQ ID NO:21, SEQ ID NO:23, or the complement of SEQ ID NO:1, SEQ ID NO:3, SEQ ID NO:5, SEQ ID NO:7, SEQ ID NO:9, SEQ ID NO:11, SEQ ID NO: 13, SEQ ID NO: 15, SEQ ID NO:17, SEQ ID NO:19, SEQ ID NO:21, or SEQ ID NO:23.

9-13. (canceled)

14. An isolated nucleic acid, the sequence of which comprises SEQ ID NO:23 or at least 100 consecutive nucleotides of SEQ ID NO:23 operably linked to a heterologous coding sequence.

15. The isolated nucleic acid of claim 14, wherein the sequence comprises SEQ ID NO:23 operably linked to a sequence selected from the group consisting of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, and SEQ ID NO:21.

16-18. (canceled)

19. An isolated nucleic acid comprising a sequence at least 95% identical to a sequence selected from the group consisting of SEQ ID NO: 3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, and SEQ ID NO:21, wherein the nucleic acid encodes a polypeptide comprising the D-hydroxymethylpentanoic acid module, the L-N-methylvaline module, the L-phenylalanine module, L-N-methylphenylalanine module, the L-proline module, the L-allo-isoleucine module, the second L-N-methylvaline module, the L-leucine module, the L-hydroxy-N-methylvaline module, or the C-terminal condensation domain, respectively, of Aureobasidin A synthetase.

20-21. (canceled)

22. An isolated nucleic acid comprising a sequence that encodes a polypeptide comprising a sequence selected from the group consisting of SEQ ID NO:4 with up to 90 amino acid insertions, deletions or substitutions, SEQ ID NO:6 with up to 149 amino acid insertions, deletions or substitutions, SEQ ID NO:8 with up to 108 amino acid insertions, deletions or substitutions, SEQ ID NO:10 with up to 149 amino acid insertions, deletions or substitutions, SEQ ID NO:12 with up to 108 amino acid insertions, deletions or substitutions, SEQ ID NO:14 with up to 108 amino acid insertions, deletions or substitutions, SEQ ID NO: 16 with up to 149 amino acid insertions, deletions or substitutions, SEQ ID NO: 18 with up to 108 amino acid insertions, deletions or substitutions, SEQ ID NO:20 with up to 149 amino acid insertions, deletions or substitutions and SEQ ID NO:22 with up to 48 amino acid insertions, deletions or substitutions, wherein the polypeptide encodes a polypeptide comprising the D-hydroxymethylpentanoic acid module, the L-N-methylvaline module, the L-phenylalanine module, the L-N-methylphenylalanine module, the L-proline module, the L-allo-isoleucine module, the second L-N-methylvaline module, the L-leucine module, the L-hydroxy-N-methylvaline module, or the C-terminal condensation domain, respectively, of Aureobasidin A synthetase or the polypeptide that catalyzes the synthesis of Aureobasidin A and related molecules.

23-25. (canceled)

26. An expression vector comprising the nucleic acid of claim 4, operably linked to an expression control sequence.

27. A recombinant vector comprising a DNA sequence encoding an enzyme that catalyzes the biosynthesis of Aureobasidin A and related molecules.

28. A cultured cell comprising the vector of claim 26.

29. The cultured cell of claim 28, or a progeny of the cell, wherein the cell expresses the polypeptide Aureobasidin A synthetase, a polypeptide that catalyzes the synthesis of Aureobasidin A, or related molecules.

30. A method of producing Aureobasidin A synthetase and related molecules, the method comprising culturing the cell of claim 29 under conditions permitting expression of the Aureobasidin A synthetase and related molecules.

31. The method of claim 30, further comprising purifying Aureobasidin A synthetase and related molecules from the cell or medium of the cell.

32. An expression vector comprising the nucleic acid of claim 8 operably linked to an expression control sequence.

33. An expression vector comprising the nucleic acid sequence of claim 22 operably linked to an expression control sequence.

34. A cultured cell comprising the vector of claim 32.

35. A cultured cell comprising the vector of claim 33.

36. (canceled)

37. A method of producing the modules of Aureobasidin A synthetase and related molecules, the method comprising culturing the cell of claim 35 under conditions permitting expression of the modules of the enzyme that catalyses the synthesis of Aureobasidin A synthetase and related molecules.

38. (canceled)

39. A purified polypeptide, comprising at least 8 consecutive residues of a a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, and SEQ ID NO:22.

40. The purified polypeptide of claim 39, an amino acid sequence at least 90% identical to a sequence selected from the group consisting of SEQ ID NO: 2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, and SEQ ID NO:22.

41. The purified polypeptide of claim 40 comprising an amino acid sequence that consists of a sequence selected from the group consisting of SEQ ID NO:2, SEQ ID NO:4, SEQ ID NO:6, SEQ ID NO:8, SEQ ID NO:10, SEQ ID NO:12, SEQ ID NO: 14, SEQ ID NO:16, SEQ ID NO:18, SEQ ID NO:20, and SEQ ID NO:22.

42. (canceled)

43. A purified polypeptide, the amino acid sequence of which comprises a sequence selected from the group consisting of SEQ ID NO:2 with 1 to 1100 conservative amino acid substitutions, SEQ ID NO:4 with up to 90 conservative amino substitutions, SEQ ID NO:6 with up to 149 conservative amino substitutions, SEQ ID NO:8 with up to 108 conservative amino substitutions, SEQ ID NO:10 with up to 149 conservative amino substitutions, SEQ ID NO:12 with up to 108 conservative amino substitutions, SEQ ID NO:14 with up to 108 conservative amino substitutions, SEQ ID NO:16 with up to 149 conservative amino substitutions, SEQ ID NO: 18 with up to 108 conservative amino substitutions, SEQ ID NO:20 with up to 149 conservative amino substitutions and SEQ ID NO:22 with up to 48 conservative amino substitutions.

44. A non-ribosomal peptide synthetase complex comprising nine biosynthetic modules arranged in the order: D-hydroxymethylpentanoic acid-L-N-methylvaline-L-phenylalanine-L-N-methylphenylalanine-L-proline-L-allo-isoleucine-L-N-methylvaline-L-leucine-L-hydroxy-N-methylvaline.

45. An expression vector comprising the nucleic acid of claim 14.

46. An expression vector comprising the nucleic acid of claim 15.

47-48. (canceled)

49. A cultured cell comprising the vector of claim 45.

50. A method of altering the expression of the aba1 gene, the method comprising
providing the cultured cell of claim 49 and
measuring the expression of the aba1 gene.
